SEQUENCE LISTING (56 sequences)

SEQ ID NO: 1 - DNA, 23 bp, artificial sequence
  primer rbc34
atgtcaccacaaacagagactaa

SEQ ID NO: 2 - DNA, 23 bp, artificial sequence
  primer rbc35
ctaaattgtatccattgccggga

SEQ ID NO: 3 - DNA, 20 bp, artificial sequence
  primer rbc36
tgcatgcaggtrtggccmcc

SEQ ID NO: 4 - DNA, 23 bp, artificial sequence
  primer rbc52
atgcaggtgtggccaccagttgg

SEQ ID NO: 5 - DNA, 24 bp, artificial sequence
  primer 53
ttagaggccgccagcaggcttgta

SEQ ID NO: 6 - DNA, 35 bp, artificial sequence
  primer rbc61
accatattcattctggaaccgtagtaggtaagctt

SEQ ID NO: 7 - DNA, 35 bp, artificial sequence
  primer rbc62
aagcttacctactacggttccagaatgaatatggt

SEQ ID NO: 8 - DNA, 35 bp, artificial sequence
  primer rbc56
ggattccttgcattgagttcgagttggaacaccca

SEQ ID NO: 9 - DNA, 35 bp, artificial sequence
  primer rbc57
tgggtgttccaactcgaactcaatgcaaggaatcc

SEQ ID NO: 10 - DNA, 30 bp, artificial sequence
  primer rbc59
ccgtggccacaaacagagactaaagcaagt

SEQ ID NO: 11 - DNA, 56 bp, artificial sequence
  primer rbc60
tagcggccgcctagtgatggtgatggtgatgaattgtatccattgccgggaattca

SEQ ID NO: 12 - DNA, 61 bp, artificial sequence
  primer rbc58
tagcggccgcttaagcataatctggaacatcatatggatagaggccgccagcaggcttgta

SEQ ID NO: 13 - DNA, 32 bp, artificial sequence
  primer rbc135
ttaccatggcaccacaaacagagactaaagca

SEQ ID NO: 14 - DNA, 29 bp, artificial sequence
  primer rbc136
ttgaattcttagtgatggtgatggtgatg

SEQ ID NO: 15 - DNA, 36 bp, artificial sequence
  primer rbc70
ccgtggccagctaactctgtagaagaacggacaagg

SEQ ID NO: 16 - DNA, 55 bp, artificial sequence
  primer rbc71
tagcggccgcttagtgatggtgatggtgatgaacattagctgttggagtttctac

SEQ ID NO: 17 - DNA, 39 bp, artificial sequence
  primer rbc66
ttggtggtggtacaattggacatccagatggaattcaag

SEQ ID NO: 18 - DNA, 39 bp, artificial sequence
  primer rbc67
cttgaattccatctggatgtccaattgtaccaccaccaa

SEQ ID NO: 19 - DNA, 39 bp, artificial sequence
  primer rbc64
caaaaatgggatattgggaccctaactatgcagtaaaag

SEQ ID NO: 20 - DNA, 39 bp, artificial sequence
  primer rbc65
cttttactgcatagttagggtcccaatatcccatttttg

SEQ ID NO: 21 - DNA, 31 bp, artificial sequence
  primer rbc80
tagcggccgcttaagcataatctggaacatc

SEQ ID NO: 22 - DNA, 41 bp, artificial sequence
  primer rbc68
ccgtggccaagattaacacaaggaactttttctttcctacc

SEQ ID NO: 23 - DNA, 63 bp, artificial sequence
  primer rbc69
tagcggccgcttaagcataatctggaacatcatatggataatatctagatccttctggcttat

SEQ ID NO: 24 - DNA, 29 bp, artificial sequence
  primer rbc134
ttaccatggctaactctgtagaagaacgg

SEQ ID NO: 25 - DNA, 1428 bp, amaranthus edulis
atgtcaccacaaacagagactaaagcaagtgttggatttaaagctggtgttaaagattaccgattgacttattatactcctgagtatgaaaccctagatactgatatcttggcagcattccgagtaagtcctcaacctggagttccacctgaagaagcgggggctgcagtagctgccgaatcttctactggtacatggacaagtgtatggaccgacggacttaccaatcttgatcgttacaaaggacgatgctacaacatcgagcccgttgctggagaagaaaatcaatatatttgttatgtagcgtatcctttagacctttttgaagaaggttctgttactaacatgtttacttccattgtgggtaacgtatttgggttcaaagctttgcgtgctctacgtttggaagatttgcgaatccctgttgcttatgtcaaaactttccaaggcccgcctcacggtatccaggttgaaagagataaattgaacaagtacggtcgtcccctattgggatgcactattaaaccaaaattggggttatccgctaaaaactatggtcgagcatgttatgaatgtcttcgtggtggacttgattttaccaaagatgatgaaaacgtgaattcccagccgttcatgcgttggagagaccgtttcctattttgtgccgaagctatttataaatcacaagccgaaacaggtgaaatcaaagggcattatttgaatgctaccgcaggtacttgcgaagaaatgataaaaagagctgtatttgctagagagttgggagttccaatcgtaatgcatgactacttaacaggtggattcactgcaaatactagcttgtctcagtattgccgagacaatggtctacttcttcacatccaccgtgcaatgcacgcagttattgatagacagaagaatcatggtatgcacttccgtgtactagctaaagcgttacgtctgtctggtggagaccatattcattctggtaccgtagtaggtaagcttgaaggggaaagagatattactttaggctttgttgatttactacgtgatgattatactgaaaaagacagaagtcgcggtatctttttcactcaatcttgggtttccacacctggtgttcttcctgttgcttcaggaggtattcacgtttggcatatgcctgctctaaccgaaatcttcggggatgattctgtactacagtttggtggaggaactttaggacacccttgggggaatgcaccgggtgctgtagcgaatcgagtagctctagaagcatgtgtacaagctcgtaatgagggacgtgaccttgctcgcgagggtaatacaattattcgtgaagctgctaaatggagtcctgaactagctgctgcttgtgaagtatggaaggaaatcaaatttgaattcccggcaatggatacaatttag

SEQ ID NO: 26 - DNA, 375 bp, amaranthus edulis
atgcaggtgtggccaccagttggtaagaagaagtttgagaccttgtcttaccttccacctctaagtgacgcacaattgttggctcaagttcaataccttctcaacaagggatggattccttgcattgaattcgagttggaacacccatttgtttaccgtgagaaccacaggtcaccaggataccaagatggacgttactggactatgtggaagttgcccatgtacggatgcactgaccccgcccaggtgttgaacgaggttgaggaagccaagaaggcttacccaagcgccttcatcagaatcattggattcgacaataaacgtcaagtgcaatgtgtcagtttcattgcttacaagcctgctggcggcctctaa

SEQ ID NO: 27 - DNA, 1623 bp, amaranthus edulis
atggcttcctctgtcatttcttcagcagctgttgccacacgcagcaatgttacacaagctagcatggttgcacctttcactggtctcaaatcttcagccactttccctgttacaaagaagcaaaaccttgacatcacttccattgctagcaatggtggaagagttagctgcatgcaggtgtggccacaaacagagactaaagcaagtgttggatttaaagctggtgttaaagattaccgattgacttattatactcctgagtatgaaaccctagatactgatatcttggcagcattccgagtaagtcctcaacctggagttccacctgaagaagcgggggctgcagtagctgccgaatcttctactggtacatggacaagtgtatggaccgacggacttaccaatcttgatcgttacaaaggacgatgctacaacatcgagcccgttgctggagaagaaaatcaatatatttgttatgtagcgtatcctttagacctttttgaagaaggttctgttactaacatgtttacttccattgtgggtaacgtatttgggttcaaagctttgcgtgctctacgtttggaagatttgcgaatccctgttgcttatgtcaaaactttccaaggcccgcctcacggtatccaggttgaaagagataaattgaacaagtacggtcgtcccctattgggatgcactattaaaccaaaattggggttatccgctaaaaactatggtcgagcatgttatgaatgtcttcgtggtggacttgattttaccaaagatgatgaaaacgtgaattcccagccgttcatgcgttggagagaccgtttcctattttgtgccgaagctatttataaatcacaagccgaaacaggtgaaatcaaagggcattatttgaatgctaccgcaggtacttgcgaagaaatgataaaaagagctgtatttgctagagagttgggagttccaatcgtaatgcatgactacttaacaggtggattcactgcaaatactagcttgtctcagtattgccgagacaatggtctacttcttcacatccaccgtgcaatgcacgcagttattgatagacagaagaatcatggtatgcacttccgtgtactagctaaagcgttacgtctgtctggtggagaccatattcattctggaaccgtagtaggtaagcttgaaggggaaagagatattactttaggctttgttgatttactacgtgatgattatactgaaaaagacagaagtcgcggtatctttttcactcaatcttgggtttccacacctggtgttcttcctgttgcttcaggaggtattcacgtttggcatatgcctgctctaaccgaaatcttcggggatgattctgtactacagtttggtggaggaactttaggacacccttgggggaatgcaccgggtgctgtagcgaatcgagtagctctagaagcatgtgtacaagctcgtaatgagggacgtgaccttgctcgcgagggtaatacaattattcgtgaagctgctaaatggagtcctgaactagctgctgcttgtgaagtatggaaggaaatcaaatttgaattcccggcaatggatacaattcatcaccatcaccatcactag

SEQ ID NO: 28 - DNA, 573 bp, amaranthus edulis
atggcttcctctgtcatttcttcagcagctgttgccacacgcagcaatgttacacaagctagcatggttgcacctttcactggtctcaaatcttcagccactttccctgttacaaagaagcaaaaccttgacatcacttccattgctagcaatggtggaagagttagctgcatgcaggtgtggccaccagttggtaagaagaagtttgagaccttgtcttaccttccacctctaagtgacgcacaattgttggctcaagttcaataccttctcaacaagggatggattccttgcattgagttcgagttggaacacccatttgtttaccgtgagaaccacaggtcaccaggataccaagatggacgttactggactatgtggaagttgcccatgtacggatgcactgaccccgcccaggtgttgaacgaggttgaggaagccaagaaggcttacccaagcgccttcatcagaatcattggattcgacaataaacgtcaagtgcaatgtgtcagtttcattgcttacaagcctgctggcggcctctatccatatgatgttccagattatgcttaa

SEQ ID NO: 29 - DNA, 1446 bp, amaranthus edulis
atggcaccacaaacagagactaaagcaagtgttggatttaaagctggtgttaaagattaccgattgacttattatactcctgagtatgaaaccctagatactgatatcttggcagcattccgagtaagtcctcaacctggagttccacctgaagaagcgggggctgcagtagctgccgaatcttctactggtacatggacaagtgtatggaccgacggacttaccaatcttgatcgttacaaaggacgatgctacaacatcgagcccgttgctggagaagaaaatcaatatatttgttatgtagcgtatcctttagacctttttgaagaaggttctgttactaacatgtttacttccattgtgggtaacgtatttgggttcaaagctttgcgtgctctacgtttggaagatttgcgaatccctgttgcttatgtcaaaactttccaaggcccgcctcacggtatccaggttgaaagagataaattgaacaagtacggtcgtcccctattgggatgcactattaaaccaaaattggggttatccgctaaaaactatggtcgagcatgttatgaatgtcttcgtggtggacttgattttaccaaagatgatgaaaacgtgaattcccagccgttcatgcgttggagagaccgtttcctattttgtgccgaagctatttataaatcacaagccgaaacaggtgaaatcaaagggcattatttgaatgctaccgcaggtacttgcgaagaaatgataaaaagagctgtatttgctagagagttgggagttccaatcgtaatgcatgactacttaacaggtggattcactgcaaatactagcttgtctcagtattgccgagacaatggtctacttcttcacatccaccgtgcaatgcacgcagttattgatagacagaagaatcatggtatgcacttccgtgtactagctaaagcgttacgtctgtctggtggagaccatattcattctggaaccgtagtaggtaagcttgaaggggaaagagatattactttaggctttgttgatttactacgtgatgattatactgaaaaagacagaagtcgcggtatctttttcactcaatcttgggtttccacacctggtgttcttcctgttgcttcaggaggtattcacgtttggcatatgcctgctctaaccgaaatcttcggggatgattctgtactacagtttggtggaggaactttaggacacccttgggggaatgcaccgggtgctgtagcgaatcgagtagctctagaagcatgtgtacaagctcgtaatgagggacgtgaccttgctcgcgagggtaatacaattattcgtgaagctgctaaatggagtcctgaactagctgctgcttgtgaagtatggaaggaaatcaaatttgaattcccggcaatggatacaattcatcaccatcaccatcactaa

SEQ ID NO: 30 - DNA, 1668 bp, Griffithsia monilis
atggcttcctctgtcatttcttcagcagctgttgccacacgcagcaatgttacacaagctagcatggttgcacctttcactggtctcaaatcttcagccactttccctgttacaaagaagcaaaaccttgacatcacttccattgctagcaatggtggaagagttagctgcatgcaggtgtggccagctaactctgtagaagaacggacaaggattaaaaatgaacgttatgaatctggtgtaattccatacgcaaaaatgggatattgggaccctaactatgcagtaaaagatactgatattctagctttatttcgtgtaagtccacaaccaggtgttgacccagtagaagcttctgctgcagttgctggtgaatcatctactgcaacatggacagtagtatggactgatttacttacagcttgtgatctatatagagcaaaagcatataaagtagagtctgttcctaatacttctgatcaatactttgcttatatctcatatgatattgacttatttgaagaaggttctattgcaaacttaacggcttctattattggtaacgtattcggttttaaagcagtaaaagctttaagactagaagatatgcgtataccagtagcttatctaaaaactttccaaggtcctgctacaggtatagttgtagaacgtgaacgtatggataaattcggtcgtccatttttaggagctactgtaaaacctaaactaggtttatctggtaaaaattatggtcgtgtagtttatgaaggtttaagaggtggattagatttcctgaaagatgatgaaaatatcaactctcaaccatttatgcgctggaaagaaagattcctatattcaatagaagctgtaaatcgttctattgctgctacaggtgaagtcaaaggacattatatgaatgtcacagctgctacaatggaagagatgtatgaaagagctgaatttgctaagcaactaggcacagttatcatcatgattgacttagtaattggatatacagctattcaaactatgggaatctgggcacgtaagaatgacatgattctgcatttacatcgtgctggtaactctacttactctcgtcaaaaaattcatggaatgaatttccgtgttatttgtaaatggatgcgtatggcaggtgtagatcatattcatgcaggtactgtagtaggaaaattagaaggtgatcctttaatgattagaggcttctataatactttattattaccatacttagaagttaatctacctcaaggtatattcttccaacaagattgggcatcattacgtaaagtaactcctgtagcttcaggtggtattcactgcggtcaaatgcatcaattattagattatcttggtaatgacgtagtattacagtttggtggtggtacaattggacatccagatggaattcaagcaggtgcaacagcaaaccgagtagctttagaatcaatggtaattgctcgcaatgaaggccgtgactatgtagcagaaggtccacaaattttacgtgatgctgcaaaaacatgtggacctttacaaacagcattagacttatggaaagatatcacatttaactatacttctacagatacagctgattttgtagaaactccaacagctaatgttcatcaccatcaccatcactaa

SEQ ID NO: 31 - protein, 555 aa, Griffithsia monilis
MASSVISSAAVATRSNVTQASMVAPFTGLKSSATFPVTKKQNLDITSIASNGGRVSCMQVWPANSVEERTRIKNERYESGVIPYAKMGYWDPNYAVKDTDILALFRVSPQPGVDPVEASAAVAGESSTATWTVVWTDLLTACDLYRAKAYKVESVPNTSDQYFAYISYDIDLFEEGSIANLTASIIGNVFGFKAVKALRLEDMRIPVAYLKTFQGPATGIVVERERMDKFGRPFLGATVKPKLGLSGKNYGRVVYEGLRGGLDFLKDDENINSQPFMRWKERFLYSIEAVNRSIAATGEVKGHYMNVTAATMEEMYERAEFAKQLGTVIIMIDLVIGYTAIQTMGIWARKNDMILHLHRAGNSTYSRQKIHGMNFRVICKWMRMAGVDHIHAGTVVGKLEGDPLMIRGFYNTLLLPYLEVNLPQGIFFQQDWASLRKVTPVASGGIHCGQMHQLLDYLGNDVVLQFGGGTIGHPDGIQAGATANRVALESMVIARNEGRDYVAEGPQILRDAAKTCGPLQTALDLWKDITFNYTSTDTADFVETPTANVHHHHHH

SEQ ID NO: 32 - DNA, 627 bp, Griffithsia monilis
atggcttcctctgtcatttcttcagcagctgttgccacacgcagcaatgttacacaagctagcatggttgcacctttcactggtctcaaatcttcagccactttccctgttacaaagaagcaaaaccttgacatcacttccattgctagcaatggtggaagagttagctgcatgcaggtgtggccaagattaacacaaggaactttttctttcctaccagatttaactgacgaacaaattaaaaagcaagttgactatgcaatatctcaaaattgggcaatcaatattgagtatactgaagatccacacccaagaaataacttctgggaattatggggattaccattatttgacattaatgacgcagcaacagtaatgtatgaaattggcagttgccgtcaacaacattcgaatgtttatatcaaagtgaatgcatttgataatacaagaggtgttgaaagttgtgttctttcatttcttatcaatagaccatcttatgaaccaggttttagattagtaagaagtgaagatatcagcagaaatcaaaaatatagcttccatagctatgcaactgataagccagaaggatctagatattatccatatgatgttccagattatgcttaa

SEQ ID NO: 33 - protein, 208 aa, Griffithsia monilis
MASSVISSAAVATRSNVTQASMVAPFTGLKSSATFPVTKKQNLDITSIASNGGRVSCMQVWPRLTQGTFSFLPDLTDEQIKKQVDYAISQNWAINIEYTEDPHPRNNFWELWGLPLFDINDAATVMYEIGSCRQQHSNVYIKVNAFDNTRGVESCVLSFLINRPSYEPGFRLVRSEDISRNQKYSFHSYATDKPEGSRYYPYDVPDYA

SEQ ID NO: 34 - DNA, 1485 bp, Griffithsia monilis
atggctaactctgtagaagaacggacaaggattaaaaatgaacgttatgaatctggtgtaattccatacgcaaaaatgggatattgggaccctaactatgcagtaaaagatactgatattctagctttatttcgtgtaagtccacaaccaggtgttgacccagtagaagcttctgctgcagttgctggtgaatcatctactgcaacatggacagtagtatggactgatttacttacagcttgtgatctatatagagcaaaagcatataaagtagagtctgttcctaatacttctgatcaatactttgcttatatctcatatgatattgacttatttgaagaaggttctattgcaaacttaacggcttctattattggtaacgtattcggttttaaagcagtaaaagctttaagactagaagatatgcgtataccagtagcttatctaaaaactttccaaggtcctgctacaggtatagttgtagaacgtgaacgtatggataaattcggtcgtccatttttaggagctactgtaaaacctaaactaggtttatctggtaaaaattatggtcgtgtagtttatgaaggtttaagaggtggattagatttcctgaaagatgatgaaaatatcaactctcaaccatttatgcgctggaaagaaagattcctatattcaatagaagctgtaaatcgttctattgctgctacaggtgaagtcaaaggacattatatgaatgtcacagctgctacaatggaagagatgtatgaaagagctgaatttgctaagcaactaggcacagttatcatcatgattgacttagtaattggatatacagctattcaaactatgggaatctgggcacgtaagaatgacatgattctgcatttacatcgtgctggtaactctacttactctcgtcaaaaaattcatggaatgaatttccgtgttatttgtaaatggatgcgtatggcaggtgtagatcatattcatgcaggtactgtagtaggaaaattagaaggtgatcctttaatgattagaggcttctataatactttattattaccatacttagaagttaatctacctcaaggtatattcttccaacaagattgggcatcattacgtaaagtaactcctgtagcttcaggtggtattcactgcggtcaaatgcatcaattattagattatcttggtaatgacgtagtattacagtttggtggtggtacaattggacatccagatggaattcaagcaggtgcaacagcaaaccgagtagctttagaatcaatggtaattgctcgcaatgaaggccgtgactatgtagcagaaggtccacaaattttacgtgatgctgcaaaaacatgtggacctttacaaacagcattagacttatggaaagatatcacatttaactatacttctacagatacagctgattttgtagaaactccaacagctaatgttcatcaccatcaccatcactaa

SEQ ID NO: 35 - protein, 494 aa, Griffithsia monilis
MANSVEERTRIKNERYESGVIPYAKMGYWDPNYAVKDTDILALFRVSPQPGVDPVEASAAVAGESSTATWTVVWTDLLTACDLYRAKAYKVESVPNTSDQYFAYISYDIDLFEEGSIANLTASIIGNVFGFKAVKALRLEDMRIPVAYLKTFQGPATGIVVERERMDKFGRPFLGATVKPKLGLSGKNYGRVVYEGLRGGLDFLKDDENINSQPFMRWKERFLYSIEAVNRSIAATGEVKGHYMNVTAATMEEMYERAEFAKQLGTVIIMIDLVIGYTAIQTMGIWARKNDMILHLHRAGNSTYSRQKIHGMNFRVICKWMRMAGVDHIHAGTVVGKLEGDPLMIRGFYNTLLLPYLEVNLPQGIFFQQDWASLRKVTPVASGGIHCGQMHQLLDYLGNDVVLQFGGGTIGHPDGIQAGATANRVALESMVIARNEGRDYVAEGPQILRDAAKTCGPLQTALDLWKDITFNYTSTDTADFVETPTANVHHHHHH

SEQ ID NO: 36 - protein, 474 aa, Amaranthus edulis
MSPQTETKASVGFKAGVKDYRLTYYTPEYETLDTDILAAFRVSPQPGVPPEEAGAAVAAESSTGTWTSVWTDGLTNLDRYKGRCYNIEPVAGEENQYICYVAYPLDLFEEGSVTNMFTSIVGNVFGFKALRALRLEDLRIPVAYVKTFQGPPHGIQVERDKLNKYGRPLLGCTIKPKLGLSAKNYGRACYECLRGGLDFTKDDENVNSQPFMRWRDRFLFCAEAIYKSQAETGEIKGHYLNATAGTCEEMIKRAVFARELGVPIVMHDYLTGGFTANTSLSQYCRDNGLLLHIHRAMHAVIDRQKNHGMHFRVLAKALRLSGGDHIHSGTVVGKLEGERDITLGFVDLLRDDYTEKDRSRGIFFTQSWVSTPGVLPVAGGIHVWHMPALTEIFGDDSVLQFGGGTLGHPWGNAPGAVANRVALEACVQARNEGRDLAREGNTIIREAAKWSPELAAACEVWKEIKFEFPAMDTI

SEQ ID NO: 37 - protein, 124 aa, Amaranthus edulis
MQVWPPVGKKKFETLSYLPPLSDAQLLAQVQYLLNKGWIPCIEFELEHPFVYRENHRSPGYQDGRYWTMWKLPMYGCTDPAQVLNEVEEAKKAYPSAFIRIIGFDNKRQVQCVSFIAYKPAGGL

SEQ ID NO: 38 - protein, 540 aa, Amaranthus edulis
MASSVISSAAVATRSNVTQASMVAPFTGLKSSATFPVTKKQNLDITSIASNGGRVSCMQVWPQTETKASVGFKAGVKDYRLTYYTPEYETLDTDILAAFRVSPQPGVPPEEAGAAVAAESSTGTWTSVWTDGLTNLDRYKGRCYNIEPVAGEENQYICYVAYPLDLFEEGSVTNMFTSIVGNVFGFKALRALRLEDLRIPVAYVKTFQGPPHGIQVERDKLNKYGRPLLGCTIKPKLGLSAKNYGRACYECLRGGLDFTKDDENVNSQPFMRWRDRFLFCAEAIYKSQAETGEIKGHYLNATAGTCEEMIKRAVFARELGVPIVMHDYLTGGFTANTSLSQYCRDNGLLLHIHRAMHAVIDRQKNHGMHFRVLAKALRLSGGDHIHSGTVVGKLEGERDITLGFVDLLRDDYTEKDRSRGIFFTQSWVSTPGVLPVASGGIHVWHMPALTEIFGDDSVLQFGGGTLGHPWGNAPGAVANRVALEACVQARNEGRDLAREGNTIIREAAKWSPELAAACEVWKEIKFEFPAMDTIHHHHHH

SEQ ID NO: 39 - protein, 190 aa, Amaranthus edulis
MASSVISSAAVATRSNVTQASMVAPFTGLKSSATFPVTKKQNLDITSIASNGGRVSCMQVWPPVGKKKFETLSYLPPLSDAQLLAQVQYLLNKGWIPCIEFELEHPFVYRENHRSPGYQDGRYWTMWKLPMYGCTDPAQVLNEVEEAKKAYPSAFIRIIGFDNKRQVQCVSFIAYKPAGGLYPYDVPDYA

SEQ ID NO: 40 - protein, 481 aa, Amaranthus edulis
MAPQTETKASVGFKAGVKDYRLTYYTPEYETLDTDILAAFRVSPQPGVPPEEAGAAVAAESSTGTWTSVWTDGLTNLDRYKGRCYNIEPVAGEENQYICYVAYPLDLFEEGSVTNMFTSIVGNVFGFKALRALRLEDLRIPVAYVKTFQGPPHGIQVERDKLNKYGRPLLGCTIKPKLGLSAKNYGRACYECLRGGLDFTKDDENVNSQPFMRWRDRFLFCAEAIYKSQAETGEIKGHYLNATAGTCEEMIKRAVFARELGVPIVMHDYLTGGFTANTSLSQYCRDNGLLLHIHRAMHAVIDRQKNHGMHFRVLAKALRLSGGDHIHSGTVVGKLEGERDITLGFVDLLRDDYTEKDRSRGIFFTQSWVSTPGVLPVASGGIHVWHMPALTEIFGDDSVLQFGGGTLGHPWGNAPGAVANRVALEACVQARNEGRDLAREGNTIIREAAKWSPELAAACEVWKEIKFEFPAMDTIHHHHHH

SEQ ID NO: 41 - DNA, 1428 bp, Zea mays
atgtcaccacaaacagaaactaaagcaagtgttggatttaaagctggtgttaaggattataaattgacttactacaccccggagtacgaaaccaaggatactgatatcttggcggcattccgagtaactccacagctcggggttccgcctgaggaagcaggggctgcagtggctgcggaatcttctgctgctggtacatggacaactgtttggactgatggacttaccagtcttgatcgttacaaaggacgatgctatcacatcgagcccgttcctggggacccagatcaatatatctgttatgtagcttatccattagacctatttgaagagggttctgttactaacatgtttacttccattgtgggtaacgtatttggtttcaaagccttacgcgctctacgtttggaggatctacgaattccccctgcttattcaaaaactttccaaggtccgccacgcggtatgcaagttgaaagggataagttgaacaagtacggtcgtcctttattgggatgtactattaaaccaaaattgggattatccgcaaaaaattacggtagagcgtgttatgagtgtctacgcggtggacttgattttaccaaagatgatgaaaacgtaaactcacaaccatttatgcgctggagagaccgtttcgtcttttgtgccgaagcaatttataaatcacaagccgaaactggtgaaatcaaggggcattacttgaatgcgactgcaggtacatgcgatgaaatgattaagggagctgtatttgcaaggcaattaggggttcctattgtaatgcatgactacttgacaggtggattcaccgcaaatactactttgtctcattattgccgcgacaacggcctacttcacattcaccgagcaatgcatgcagttattgatagacagaaaaatcatggtatgcatttccgtgtattagctaaagcattgcgtatgtcggggggagatcatatccactccggtacagtagtaggtaagttagaaggggaacgcgaaataactttaggttttgttgatttattgcgcgatgattttattgaaaaagatcgttctcgcggtatctttttcactcaggactgggtatccatgccaggtgttataccggtggcttctgggggtattcatgtttggcatatgccagctctgaccgaaatccttggagatgattcagtattacaatttggtggaggaactttaggacatccttggggaaatgcacatggtgcagcagctaatcgtgtagctttagaagcctgtgtacaagctcgtaacgaagggcgcgatctagctcgcgaggtacaaattatcaaagcagcttgcaaatggagtgccgaactagccgcagcttgcgaaatatggaaggagatcaaattcgatggtttcaaagcgatggataccatataa

SEQ ID NO: 42 - protein, 475 aa, Zea mays
MSPQTETKASVGFKAGVKDYKLTYYTPEYETKDTDILAAFRVTPQLGVPPEEAGAAVAAESSAAGTWTTVWTDGLTSLDRYKGRCYHIEPVPGDPDQYICYVAYPLDLFEEGSVTNMFTSIVGNVFGFKALRALRLEDLRIPPAYSKTFQGPPRGMQVERDKLNKYGRPLLGCTIKPKLGLSAKNYGRACYECLRGGLDFTKDDENVNSQPFMRWRDRFVFCAEAIYKSQAETGEIKGHYLNATAGTCDEMIKGAVFARQLGVPIVMHDYLTGGFTANTTLSHYCRDNGLLHIHRAMHAVIDRQKNHGMHFRVLAKALRMSGGDHIHSGTVVGKLEGEREITLGFVDLLRDDFIEKDRSRGIFFTQDWVSMPGVIPVASGGIHVWHMPALTEILGDDSVLQFGGGTLGHPWGNAHGAAANRVALEACVQARNEGRDLAREVQIIKAACKWSAELAAACEIWKEIKFDGFKAMDTI

SEQ ID NO: 43 - DNA, 513 bp, Zea mays
atggcgcccaccgtgatgatggcctcgtcggcaaccgccgtcgccccgttccagggtctcaagtccgccgccagcctccccgtcgcccgccgcagcaccaggagcctcggcaacgtcagcaacggcggaaggatccggtgcatgcaggtgtggccggcctacggcaacaagaagttcgagacgctgtcgtacctgccgccgctgtcgacggacgacctgctgaagcaggtggactacctgctgcgcaacggctggataccctgcctcgagttcagcaagctcgggttcgtgtaccgcgagaactccacctccccgtgctactacgacggccgctactggaccatgtggaagctgcccatgttcggctgcaccgacgccacgcaggtgtacaaggagctgcaggaggccatcgccgcgtacccggacgccttccaccgcgtcatcggcttcgacaacgtcaggcagacgcagtgcgtcagcttcatcgcctacaagccccccggcagcgagtag

SEQ ID NO: 44 - protein, 170 aa, Zea mays
MAPTVMMASSATAVAPFQGLKSAASLPVARRSTRSLGNVSNGGRIRCMQVWPAYGNKKFETLSYLPPLSTDDLLKQVDYLLRNGWIPCLEFSKLGFVYRENSTSPCYYDGRYWTMWKLPMFGCTDATQVYKELQEAIAAYPDAFHRVIGFDNVRQTQCVSFIAYKPPGSE

SEQ ID NO: 45 - DNA, 1431 bp, Saccharum officinarum
atgtcaccacaaacagaaactaaagcaagtgttggatttaaagctggtgttaaggattataaattgacttactacaccccggagtacgaaaccaaggatactgatatcttggcagcattccgagtaactcctcagctcggggttccgcctgaagaagcaggagctgcagtagctgcggaatcttctactggtacatggacaactgtttggactgatggacttaccagtcttgatcgttacaaaggacgatgctatcacatcgagcccgttcctggggacccagatcaatatatctgttatgtagcttatccattagacctatttgaagagggttctgttactaacatgtttacttccattgtgggtaacgtatttggtttcaaagccttacgcgctctacgtttggaggatctacgaattccccctgcttatgtaaaaactttccaaggtccgcctcacggtatccaagttgaaagggataagttgaacaagtacggtcgtcctttattgggatgtactattaaaccaaaattgggattatccgcaaaaaattacggtagagcgtgttatgagtgtctacgcggtggacttgattttaccaaagatgatgaaaacgtaaactcacaaccatttatgcgctggagagaccgttttgtcttttgtgccgaagcaatttataaagcacaagccgaaaccggtgaaatcaaggggcattacttgaatgcgactgcaggtacatgcgaagaaatgattaagagagctgtatttgcaaaggaattaggggttcctattgtaatgcatgactacttaacaggaggattcaccgcaaatactactttgtctcattattgccgcgacaacggcctacttcttcacattcaccgagcaatgcatgcagttattgatagacagaaaaatcatggtatgcatttccgtgtattagctaaagcattgcgtatgtcggggggagatcatatccactccggtacagtagtaggtaagttagaaggggaacgcgaaataactttaggttttgttgatttattgcgcgatgattttattgaaaaagatcgttctcgcggtatctttttcactcaggactgggtatccatgccaggtgttataccggtggcttcagggggtattcatgtttggcatatgccagctctgaccgaaatctttggagatgattccgtattacaatttggtggaggaactttaggacatccttggggaaatgcacctggtgcagcagctaatcgtgtggctttagaagcctgtgtacaagctcgtaacgaagggcgcgatcttgctcgtgaaggtaatgaaattatcaaagcagcttgcaaatggagtgctgaactagccgcagcttgtgaaatatggaaggagatcaaatttgatactttcaaagcgatggataccttatag

SEQ ID NO: 46 - protein, 476 aa, Saccharum officinarum
MSPQTETKASVGFKAGVKDYKLTYYTPEYETKDTDILAAFRVTPQLGVPPEEAGAAVAAESSTGTWTTVWTDGLTSLDRYKGRCYHIEPVPGDPDQYICYVAYPLDLFEEGSVTNMFTSIVGNVFGFKALRALRLEDLRIPPAYVKTFQGPPHGIQVERDKLNKYGRPLLGCTIKPKLGLSAKNYGRACYECLRGGLDFTKDDENVNSQPFMRWRDRFVFCAEAIYKAQAETGEIKGHYLNATAGTCEEMIKRAVFAKELGVPIVMHDYLTGGFTANTTLSHYCRDNGLLLHIHRAMHAVIDRQKNHGMHFRVLAKALRMSGGDHIHSGTVVGKLEGEREITLGFVDLLRDDFIEKDRSRGIFFTQDWVSMPGVIPVASGGIHVWHMPALTEIFGDDSVLQFGGGTLGHPWGNAPGAAANRVALEACVQARNEGRDLAREGNEIIKAACKWSAELAAACEIWKEIKFDTFKAMDTL

SEQ ID NO: 47 - DNA, 507 bp, Saccharum officinarum
atggcgctcaccgtgatggcctcgtccgccaccgccgctgctccgttccaggggctcaagtccaccgccagcctccccgtcgcccgacgctccaccaccagcctcgccaaagtcagcaacggcggaaggatccggtgcatgcaggtgtggccggcgtacggcaacaagaagttcgagacgctgtcgtacctgccgccgctgacgcaggagcagctgctgaagcaggtggactacctgctgcgcaacaactgggtgccctgcctcgagttcagcaaggaaggcttcgtgtaccgcgagaactccacctccccgtgctactacgacggccggtactggaccatgtggaagctgcccatgttcggctgcaccgacgcgtcgcaggtgtacaaggagctgcaggaggccatcgcgtcctacccggagctacgtgccatcctcggcttcgacaacatcaggcagacgcagtggctcacgttcatcgcctacaagcccgcgggcagcgagtaa

SEQ ID NO: 48 - protein, 168 aa, Saccharum officinarum
MALTVMASSATAAAPFQGLKSTASLPVARRSTTSLAKVSNGGRIRCMQVWPAYGNKKFETLSYLPPLTQEQLLKQVDYLLRNNWVPCLEFSKEGFVYRENSTSPCYYDGRYWTMWKLPMFGCTDASQVYKELQEAIASYPELRAILGFDNIRQTQWLTFIAYKPAGSE

SEQ ID NO: 49 - DNA, 1428 bp, Amaranthus hypochondriacus
atgtcaccacaaacagagactaaagcaagtgttggatttaaagctggtgttaaagattaccgattgacttattatactcctgagtatgaaacccaagatactgatatcttggcagcattccgagtaagtcctcaacctggagttccacctgaagaagcgggggctgcagtagctgccgaatcttctactggtacatggacaagtgtatggaccgacggacttaccaatcttgatcgttacaaaggacgatgctacaacatcgagcccgttgctggagaagaaaatcaatatatttgttatgtagcgtatcctttagacctttttgaagaaggttctgttactaacatgtttacttccattgtgggtaacgtatttgggttcaaagctttgcgtgctctacgtttggaagatttgcgaatccctgttgcttatgtcaaaactttccaaggcccgcctcacggtatccaggttgaaagagataaattgaacaagtacggtcgtcccctattgggatgcactattaaaccaaaattggggttatccgctaaaaactatggtcgagcatgttatgaatgtcttcgtggtggacttgattttaccaaagatgatgaaaacgtgaattcccagccgttcatgcgttggagagaccgtttcctattttgtgccgaagctatttataaatcacaagccgaaacaggtgaaatcaaagggcattatttgaatgctaccgcaggtacttgcgaagaaatgataaaaagagctgtatttgctagagagttgggagttccaatcgtaatgcatgactacttaacaggtggattcactgcaaatactagcttgtctcagtattgccgagacaatggtctacttcttcacatccacgtcgcaatgcacgcagttattgatagacagaagaatcatggtatgcacttccgtgtactagctaaagcgttacgtctgtctggtggagaccatattcattctggtaccgtagtaggtaagcttgaaggggaaagagatattactttaggctttgttgatttactacgtgatgattatacagaaaaagacagatgtcgcggtatctttttcactcaatcttgggtttccacacctggtgttcttcctgttgcttcaggaggtattcacgtttggcatatgcctgctctaaccgaaatcttcggggatgattctgtactacagtttggtggaggaactttaggacacccttgggggaatgcaccgggtgctgtagcgaatcgagtagctctagaagcatgtgtacaagctcgtaatgagggacgtgaccttgctcgcgagggtaatacaattattcgtgaagctgctaaatggagtcctgaactagctgctgcttgtgaagtatggaaggaaatcaaatttgaattcccggcaatggatacaatttag

SEQ ID NO: 50 - protein, 475 aa, Amaranthus hypochondriacus
MSPQTETKASVGFKAGVKDYRLTYYTPEYETQDTDILAAFRVSPQPGVPPEEAGAAVAAESSTGTWTSVWTDGLTNLDRYKGRCYNIEPVAGEENQYICYVAYPLDLFEEGSVTNMFTSIVGNVFGFKALRALRLEDLRIPVAYVKTFQGPPHGIQVERDKLNKYGRPLLGCTIKPKLGLSAKNYGRACYECLRGGLDFTKDDENVNSQPFMRWRDRFLFCAEAIYKSQAETGEIKGHYLNATAGTCEEMIKRAVFARELGVPIVMHDYLTGGFTANTSLSQYCRDNGLLLHIHVAMHAVIDRQKNHGMHFRVLAKALRLSGGDHIHSGTVVGKLEGERDITLGFVDLLRDDYTEKDRCRGIFFTQSWVSTPGVLPVASGGIHVWHMPALTEIFGDDSVLQFGGGTLGHPWGNAPGAVANRVALEACVQARNEGRDLAREGNTIIREAAKWSPELAAACEVWKEIKFEFPAMDTI

SEQ ID NO: 51 - DNA, 555 bp, Amaranthus hypochondriacus
atggcttcaagcatgatgagcaacgctgctactgccgtcgccgtcgccgccacttccggcggtgctcaggctaacatggtggcaccattcaatggtttgaagtctattgctagtttccctgttactaggaagagtaatgacattacttctattgctagtaacggtggaagagtccaatgcatgcaggtatggcccccagttggaaagaagaagttcgagaccttatcttacctcccacctcttagcgacgcacaattgttggctcaagttcaataccttctcaacaagggatggattccttgcattgagttcgagttggaacatccatttgtttaccgtgagaaccacaggtcaccaggataccaagatggacgttactggactatgtggaagttgcccatgtacgggtgcactgaccccgcccaggtgttgaatgaggtagaggaagccaagaaggcttaccctaccgctttcattagaatcattggattcgacaataaacgtcaagtgcaatgtgtcagtttcattgcctacaagcctgctgacagctactaa

SEQ ID NO: 52 - protein, 184 aa, Amaranthus hypochondriacus
MASSMMSNAATAVAVAATSGGAQANMVAPFNGLKSIASFPVTRKSNDITSIASNGGRVQCMQVWPPVGKKKFETLSYLPPLSDAQLLAQVQYLLNKGWIPCIEFELEHPFVYRENHRSPGYQDGRYWTMWKLPMYGCTDPAQVLNEVEEAKKAYPTAFIRIIGFDNKRQVQCVSFIAYKPADSY

SEQ ID NO: 53 - DNA, 1467 bp, Griffithsia monilis
atgtctaactctgtagaagaacggacaaggattaaaaatgaacgttatgaatctggtgtaattccatacgcaaaaatgggatattgggatcctaactatgcagtaaaagatactgatattctagctttatttcgtgtaagtccacaaccaggtgttgacccagtagaagcttctgctgcagttgctggtgaatcatctactgcaacatggacagtagtatggactgatttacttacagcttgtgatctatatagagcaaaagcatataaagtagagtctgttcctaatacttctgatcaatactttgcttatatctcatatgatattgacttatttgaagaaggttctattgcaaacttaacggcttctattattggtaacgtattcggttttaaagcagtaaaagctttaagactagaagatatgcgtataccagtagcttatctaaaaactttccaaggtcctgctacaggtatagttgtagaacgtgaacgtatggataaattcggtcgtccatttttaggagctactgtaaaacctaaactaggtttatctggtaaaaattatggtcgtgtagtttatgaaggtttaagaggtggattagatttcctgaaagatgatgaaaatatcaactctcaaccatttatgcgctggaaagaaagattcctatattcaatagaagctgtaaatcgttctattgctgctacaggtgaagtcaaaggacattatatgaatgtcacagctgctacaatggaagagatgtatgaaagagctgaatttgctaagcaactaggcacagttatcatcatgattgacttagtaattggatatacagctattcaaactatgggaatctgggcacgtaagaatgacatgattctgcatttacatcgtgctggtaactctacttactctcgtcaaaaaattcatggaatgaatttccgtgttatttgtaaatggatgcgtatggcaggtgtagatcatattcatgcaggtactgtagtaggaaaattagaaggtgatcctttaatgattagaggcttctataatactttattattaccatacttagaagttaatctacctcaaggtatattcttccaacaagattgggcatcattacgtaaagtaactcctgtagcttcaggtggtattcactgcggtcaaatgcatcaattattagattatcttggtaatgacgtagtattacagtttggtggtggtacaattggccatccagatggaattcaagcaggtgcaacagcaaaccgagtagctttagaatcaatggtaattgctcgcaatgaaggccgtgactatgtagcagaaggtccacaaattttacgtgatgctgcaaaaacatgtggacctttacaaacagcattagacttatggaaagatatcacatttaactatacttctacagatacagctgattttgtagaaactccaacagctaatgtttaa

SEQ ID NO: 54 - protein, 488 aa, Griffithsia monilis
MSNSVEERTRIKNERYESGVIPYAKMGYWDPNYAVKDTDILALFRVSPQPGVDPVEASAAVAGESSTATWTVVWTDLLTACDLYRAKAYKVESVPNTSDQYFAYISYDIDLFEEGSIANLTASIIGNVFGFKAVKALRLEDMRIPVAYLKTFQGPATGIVVERERMDKFGRPFLGATVKPKLGLSGKNYGRVVYEGLRGGLDFLKDDENINSQPFMRWKERFLYSIEAVNRSIAATGEVKGHYMNVTAATMEEMYERAEFAKQLGTVIIMIDLVIGYTAIQTMGIWARKNDMILHLHRAGNSTYSRQKIHGMNFRVICKWMRMAGVDHIHAGTVVGKLEGDPLMIRGFYNTLLLPYLEVNLPQGIFFQQDWASLRKVTPVASGGIHCGQMHQLLDYLGNDVVLQFGGGTIGHPDGIQAGATANRVALESMVIARNEGRDYVAEGPQILRDAAKTCGPLQTALDLWKDITFNYTSTDTADFVETPTANV

SEQ ID NO: 55 - DNA, 417 bp, Griffithsia monilis
gtgagattaacacaaggaactttttctttcctaccagatttaactgacgaacaaattaaaaagcaagttgactatgcaatatctcaaaattgggcaatcaatattgagtatactgaagatccacacccaagaaataacttctgggaattatggggattaccattatttgacattaatgacgcagcaacagtaatgtatgaaattggcagttgccgtcaacaacattcgaatgtttatatcaaagtgaatgcatttgataatacaagaggtgttgaaagttgtgttctttcatttcttatcaatagaccatcttatgaaccaggttttagattagtaagaagtgaagatatcagcagaaatcaaaaatatagcttccatagctatgcaactgataagccagaaggatctagatattaa

SEQ ID NO: 56 - protein, 138 aa, Griffithsia monilis
MRLTQGTFSFLPDLTDEQIKKQVDYAISQNWAINIEYTEDPHPRNNFWELWGLPLFDINDAATVMYEIGSCRQQHSNVYIKVNAFDNTRGVESCVLSFLINRPSYEPGFRLVRSEDISRNQKYSFHSYATDKPEGSRY

(19) United States
(12) Patent Application Publication
(10) Pub. No.: US 2009/0172842 A1
(43) Pub. Date: Jul. 2, 2009

(54) EXPRESSION OF RECOMBINANT GENES ENCODING RUBISCO PROTEINS IN C3 PLANTS

(76) Inventors: TIMOTHY CASPAR, Yorklyn, DE (US); Theodore Mitchell Klein, Wilmington, DE (US); Min Qi, Hockessin, DE (US); Jianjun Yang, Hockessin, DE (US)

Correspondence Address: E I DU PONT DE NEMOURS AND COMPANY;LEGAL PATENT RECORDS CENTER, BARLEY MILL PLAZA 25/1122B, 4417 LANCASTER PIKE, WILMINGTON, DE, 19805, US

(21) Appl. No.: 12/203,993

(22) Filed: Sep. 4, 2008

Related U.S. Application Data
(60) Provisional application No. 61/017,416, filed on Dec. 28, 2007.

(51) Int. Cl. A01H 5/00; C12N 15/82; C07K 14/00; C07H 21/00

(52) U.S. Cl. 800/295; 435/468; 530/324; 536/23.6

(57) ABSTRACT

The invention describes isolation and functional expression of RUBISCO genes derived from C4 plants and red algae in C3 plant hosts. Specifically the RUBISCO genes of Amaranthus edulis were functionally expressed at high levels in the transgenic crop plants soybean and tobacco, while the RUBISCO gene from Griffithsia monilis alga was expressed at lower levels in the transgenic crop tobacco.

This application claims the benefit of U.S. Provisional Application 61/017416, filed Dec. 28, 2007.

### FIELD OF THE INVENTION

The invention relates to the field of molecular biology and plant genetics. More specifically genes encoding RUBISCO enzymes isolated from C4 plants and rhodophytes have been expressed at high levels in C3 plants.

### BACKGROUND OF THE INVENTION

The study of ribulose-1,5-bisphosphate carboxylase/oxygenase (Rubisco, E. C. 4.1.1.39) “RUBISCO”, has been extensive as this enzyme catalyzes a key, limiting reaction in the photosynthetic process. Modulations in the activity and effectiveness of this enzyme will affect plant growth and yield, and hold the potential for increasing crop production in a variety of food plants. The RUBISCO enzymes of C4 plants and Rhodophytes are typically more efficient than those of C3 plants and engineering a C4 or Rhodophyte RUBISCO into a C3 plant with efficient expression would represent an advance in the art. To date the obstacles to effecting this recombinant construction have been legion.

As noted, the function of RUBISCO is crucial to the photosynthetic process. RUBISCO catalyzes the carboxylation of ribulose-1,5-bisphosphate (RuBP) producing two molecules of 3-phosphoglycerate (PGA), which are partly utilized in the Calvin cycle to regenerate the carbon dioxide acceptor RuBP and partly converted to carbohydrate which supports plant growth. This pathway is responsible for the annual net fixation of 1011 tons of CO₂ into the biosphere, a process upon which all agriculture ultimately depends. In addition to carboxylation of RuBP, RUBISCO also catalyzes its oxygenation, producing one molecule of PGA and one molecule of phosphoglycolate from each molecule of RuBP. The PGA is recycled through the Calvin cycle but the phosphoglycolate is metabolized by the photorespiratory pathway. This pathway utilizes energy in the form of ATP and reducing equivalents to recycle three quarters of the carbon in the phosphoglycolate back to PGA. However, for each molecule of RuBP which is oxygenated, one half molecule of CO₂ is released during photorespiration. The oxygenation reaction of RuBP performed by RUBISCO has no widely accepted value to the plant. Similarly, with the exception of recycling phosphoglycolate back into PGA, the photorespiratory pathway also has no known value to the plant.

The RUBISCO enzyme from plants is a sub-optimal enzyme because of its low catalytic activity, poor ability to discriminate between CO₂ and O₂, (Andrews, T. J., Whitney, S. M., Arch Biochem Biophys, 414, 159-169, 2003).

Models which relate RUBISCO parameters to photosynthesis, growth, and yield have been developed (von Caemmerer, S., Biochemical Models of Leaf Photosynthesis 2000, CSIRO Publishing; Zhu, X. -G., et al., Plant Cell and Environment, 27,155-165, 2004; Alagarswamy, G., et al. Agron. J., 98, 34-42, 2006; and Whitney, S. M. and Andrews, T. J., Plant Physiol., 133, 287-294, 2003). These models predict that increasing RUBISCO'S catalytic efficiency will result in a substantial increase in plants' productivity. In particular, if the oxygenase activity were eliminated and the rate of carboxylation increased about ten-fold, plant productivity would be predicted to increase by 50%. A RUBISCO with better kinetic properties than the version in a particular plant could be identified from other plant or non-plant sources. Alternatively, an improved RUBISCO enzyme could be created by rational protein design and/or in vitro evolution e.g. U.S. patent application Ser. No. 09/437,726 and US patent No. 2006/0117409A1.

Amaranthus edulis is a dicot C4 plant and its RUBISCO has better kinetic properties (e.g., k^{c}_{cat} of 7.3 and Tao of 82 (Seemann, J. R., et al., Plant Physiol., 74, 791-794, 1984 and Zhu. X. -G., et al., Plant Cell Environ., 27, 155-165, 2004) compared to major C3 crops such as soybean and tobacco. It therefore is possible to increase the efficiency of photosynthesis in C3 plants through expression of the A. edulis RUBISCO in them. Based on a photosynthetic model, complete replacement of A. edulis RUBISCO could increase photosynthesis by 17% in an average C3 plant (Zhu. X. -G., et al., supra). To date, no attempts to express a C4 Rubisco in a C3 plant have been reported.

Griffithsia monilis is a rhodophyte (red alga) which also has a RUBISCO enzyme (Genbank accession #EU079379.1) with much improved kinetic properties, k^{c}_{cat} of 2.6, tau of 167 (Whitney S. M., et al., Plant J., 26, 535-547, 2001) relative to higher plants. Based on these kinetic properties, modeling indicates that if the endogenous RUBISCO in C3 plants could be replaced with a functional version of this enzyme, then the photosynthesis of the plant would be improved by 27% (Zhu. et al., supra). To date, all attempts to express algal L8S8 RUBISCO genes in plants have been unsuccessful, producing only insoluble, inactive protein (Whitney S. M., et al., Plant J., 26, 535-547, 2001).

The problem to be solved is therefore to achieve functional expression of RUBISCO genes with kinetically improved properties relative to C3 plants, e.g., from A. edulis and G. monilis, in the chloroplasts of soybean and tobacco plants for better crop performance.

### SUMMARY OF THE INVENTION

The invention relates to the expression of the genes encoding a C4 plant or rhodophyte derived RUBISCO enzyme in a C3 plant. Specifically the RUBISCO genes of Amaranthus edulis and Griffithsia monilis were functionally expressed in the transgenic crop plants soybean and tobacco.

Accordingly the invention provides a method for the recombinant expression of an L8S8 RUBISCO enzyme in a plant cell comprising:
a) providing a C3 plant cell comprising a transformation vector wherein the vector comprises a heterologous genetic construct encoding a plant protein selected from the group consisting of: the small subunit of a L8S8 RUBISCO enzyme and the large subunit of an L8S8 RUBISCO enzyme, wherein the large and small subunits of the L8S8 RUBISCO enzyme are derived from a C4 plant or a rhodophyte; and
b) growing the plant cell under conditions whereby the protein is expressed in soluble form.

In a preferred embodiment the invention utilizes a chloroplast vector essentially of the general structure:
hetero Pro1::M:: Tern hetero Pro2::RBC::Ter2

Wherein:
a) hetero Pro1 is a promoter derived from a non-RUBISCO plant gene;
b) M genetic construct encoding a selectable marker;
c) Tern is a terminator;
d) hetero Pro2 is a promoter derived from a non-RUBISCO plant gene;
e) RBC is a genetic construct encoding a plant protein selected from the group consisting of: the small subunit of a L8S8 RUBISCO enzyme and the large subunit of an L8S8 RUBISCO enzyme, wherein the large and small subunits of the L8S8 RUBISCO enzyme are derived from a C4 plant or a rhodophyte; and
f) Ter2 is a terminator.

In another embodiment the invention provides a C3 plant comprising a soluble plant protein selected from the group consisting of: the small subunit of a L8S8 RUBISCO enzyme and the large subunit of an L8S8 RUBISCO enzyme, wherein the large and small subunits of the L8S8 RUBISCO enzyme are derived from Amaranthus or Griffithsia.

In another embodiment the invention provides a polypeptide encoding a large subunit of an L8S8 RUBISCO enzyme selected from the group consisting of SEQ ID NO: 36, 38 and 40.

Similarly the invention provides an isolated nucleic acid sequence encoding a large subunit of an L8S8 RUBISCO enzyme having a nucleic sequence selected from the group consisting of SEQ ID NO: 25, 27 and 29.

Additionally the invention provides a polypeptide encoding a small subunit of an L8S8 RUBISCO enzyme selected from the group consisting of SEQ ID NO: 37 and 39.

Similarly the invention provides An isolated nucleic acid sequence encoding a small subunit of an L8S8 RUBISCO enzyme having a nucleic sequence selected from the group consisting of SEQ ID NO: 26 and 28.

### BRIEF DESCRIPTION OF THE FIGURES AND SEQUENCE LISTING

The invention can be more fully understood from the following detailed description and the accompanying drawings and Sequence Listing which form a part of this application.

FIG. 1. Plasmid Map of pREC102.

FIG. 2. Plasmid Map of pREC1104

FIG. 3. Plasmid Map of pRBI104-LB and RB are left border and right border sequences of T-DNA in aTi plasmid of Agrobacterium

FIG. 4. Plasmid Map of pRBI105.

FIG. 5. Plasmid Map of pRBI106.

FIG. 6. SDS-PAGE immunoblot assay. (A) Protein extracts of pRBI104 tobacco transformants were probed with Anti-His(C-term)-HRP Ab. (B) Protein extracts of PRBI105 tobacco transformants were probed with Anti-HA-HRP Ab. (C) Protein extracts of pRBI106 tobacco transformants were probed with Anti-His(C-term)-HRP Ab. (D) Protein extracts of PRBI106 tobacco transformants were probed with Anti-HA-HRP Ab. Each sample contained 6 μg protein, except for the standards used for quantification for which 140 ng of GST-His and 14 ng of GST-HA was used. Sample IDs are indicated on the top. WT: protein extract of wild-type tobacco. Standard protein MW ladder is shown on the left. LSU or SSU is marked on the right.

FIG. 7. Native-PAGE immunoblot assay. (A) Protein extracts of pRBI104 tobacco transformants were probed with Anti-His(C-term)-HRP Ab. (B) Protein extracts of PRBI105 tobacco transformants were probed with Anti-HA-HRP Ab. (C) Protein extracts of pRBI106 tobacco transformants were probed with Anti-His(C-term)-HRP Ab. (D) Protein extracts of PRBI106 tobacco transformants were probed with Anti-HA-HRP Ab. Each sample contained 6 μg protein. Sample IDs are indicated on the top. WT: protein extract of wild-type tobacco. Standard protein MW ladder is shown on the left. L8S8 complex is marked on the right.

FIG. 8. Plasmid Map of pRST106

FIG. 9. Plasmid Map of pRST108.

FIG. 10. Plasmid Map of pRST107.

FIG. 11. SDS-PAGE immunoblot assay. (A) Protein extracts of pRST107 soy transformants were probed with Anti-His(C-term)-HRP Ab. (B) Protein extracts of pRST107 soy transformants were probed with Anti-HA-HRP Ab. (C) Protein extracts of pRST108 soy transformants were probed with Anti-HA-HRP Ab. Each sample contained 6 μg protein, except for the standards used for quantification for which 140 ng of GST-His and 14 ng of GST-HA was used. Sample IDs are indicated on the top. WT: protein extract of wild-type tobacco. Standard protein MW ladder is shown on the left. LSU or SSU is marked on the right.

FIG. 12. Native-PAGE immunoblot assay. (A) Protein extracts of pRST107 tobaccos were probed with Anti-His(C-term)-HRP Ab. (B) Protein extracts of pRST107 tobaccos were probed with Anti-HA-HRP Ab. (C) Protein extracts of pRST108 tobaccos were probed with Anti-HA-HRP Ab. Each sample contained 6 μg protein. Sample IDs are indicated on the top. WT: protein extract of wild-type tobacco. Standard protein MW ladder is shown on the left. L8S8 complex is marked on the right.

FIG. 13. Plasmid Map of pTCP103.

FIG. 14. SDS-PAGE immunoblot assay. Leaf protein extracts of tobacco plants transformed with pTCP103 were probed with Anti-His(C-term)-HRP Ab. Each sample contained 5 μg protein, except for the standards for which 140 ng of the GST-His were used. Sample IDs are indicated on the top. KO: protein extract of rbcL-KO tobacco. Standard protein MW ladder is shown on the left. LSU is marked on the right.

FIG. 15. Native-PAGE immunoblot assay. Leaf protein extracts of tobacco plants transformed with pTCP103 were probed with Anti-His(C-term)-HRP Ab. Each sample contained 5 μg protein. Sample IDs are indicated on the top. WT: protein extract of wild-type tobacco. KO: protein extract of rbcL-KO tobacco. Standard protein MW ladder is shown on the left. The location of the L8S8 holoenzyme complex is shown on the right.

FIG. 16—Immunoblot assay of the purified RUBISCO from pTCP103-1 tobacco. Proteins were separated by SDS-PAGE (upper panels) and Native-PAGE (lower panels) and probed with Anti-His (C-term)-HRP Ab (A) and Anti-NTrbcS Ab (B). Samples are indicated on the top and contained 5 μg protein, except that WT has 2.5 μg protein and AE-P has 2 μg protein. WT: protein extract of wild-type tobacco. KO: protein extract of rbcL-KO tobacco. AE-C: protein extract of pTCP103-1 tobacco. AE-P: Ni-NTA purified protein of pTCP103-1 tobacco. RUBISCO LSU, SSU, and L8S8 complex are marked on the right

FIG. 17 Plasmid Map of pREC103.

FIG. 18 Plasmid Map of pRECI105

FIG. 19 Plasmid Map of pRBI107.

FIG. 20 Plasmid Map of pRBI108.

FIG. 21 Plasmid Map of pRBI109.

FIG. 22 Plasmid Map of pRST109.

FIG. 23 Plasmid Map of pRST110

FIG. 24 Plasmid Map of pRST111.

FIG. 25 Plasmid Map of pTCP104.

FIG. 26 SDS-PAGE immunoblot assay. Protein extracts of pTCP104 transformants were probed with Anti-His(C-term)-HRP Ab. Each sample contained 5 μg protein, except the standard lane which contained 140 ng GST-His. Sample IDs are indicated at the top. WT: protein extract of wild-type tobacco. KO: protein extract of rbcL-KO tobacco. Standard protein MW ladder is shown on the left. The location of LSU is marked on the right.

FIG. 27 Immunoblot assay of the purified Rubisco from pTCP104-1 tobacco. Proteins were separated by SDS-PAGE (upper panels) and Native-PAGE (lower panels) and probed with Anti-His (C-term)-HRP Ab (A) and Anti-rbcS Ab (B). Samples are indicated on the top. WT: crude soluble protein extract of wild-type tobacco (2.5 μg protein). GST-His: 140 ng purified GST-His. GMN-C: crude soluble protein extract of pTCP104-1 Tobacco (5 μg protein). GMN-P: Ni-NTA purified protein of pTCP104-1 tobacco (2 μg protein). The locations of Rubisco LSU, SSU, and L8S8 complex are marked on the right.

The sequence descriptions and Sequence Listing attached hereto comply with the rules governing nucleotide and/or amino acid sequence disclosures in patent applications as set forth in 37 C.F.R. §1.821-1.825.

The Sequence Listing contains the one letter code for nucleotide sequence characters and the three letter codes for amino acids as defined in conformity with the IUPAC-IUBMB standards described in (Nucleic Acids Res. 13, 3021-3030, 1985 and Biochem. J., 219, 345-373, 1984) which are herein incorporated by reference. The symbols and format used for nucleotide and amino acid sequence data comply with the rules set forth in 37 C.F.R. §1.822. sequences of the invention are summarized below.

TABLE 1
PRIMERS

| | | |
|---|---|---|
| SEQ | Primer | |
| ID NO | name | Sequence |

| | | |
|---|---|---|
| 1 | rbc34 | ATGTCACCACAAACAGAGACTAA |
| 2 | rbc35 | CTAAATTGTATCCATTGCCGGGA |
| 3 | rbc36 | TGCATGCAGGTRTGGCCMCC |
| 4 | rbc52 | ATGCAGGTGTGGCCACCAGTTGG |
| 5 | rbc53 | TTAGAGGCCGCCAGCAGGCTTGTA |
| 6 | rbc61 | ACCATATTCATTCTGGAACCGTAGTAGGTAAGCTT |
| 7 | rbc62 | AAGCTTACCTACTACGGTTCCAGAATGAATATGGT |
| 8 | rbc56 | GGATTCCTTGCATTGAGTTCGAGTTGGAACACCCA |
| 9 | rbc57 | TGGGTGTTCCAACTCGAACTCAATGCAAGGAATCC |
| 10 | rbc59 | CCGTGGCCACAAACAGAGACTAAAGCAAGT |
| 11 | rbc60 | TAGCGGCCGCCTAGTGATGGTGATGGTGATGAATTGT |
| | | ATCCATTGCCGGGAATTCA |
| 12 | rbc58 | TAGCGGCCGCTTAAGCATAATCTGGAACATCATATGG |
| | | ATAGAGGCCGCCAGCAGGCTTGTA |
| 13 | rbc135 | TTACCATGGCACCACAAACAGAGACTAAAGCA |
| 14 | rbc136 | TTGAATTCTTAGTGATGGTGATGGTGATG |
| 15 | rbc70 | CCGTGGCCAGCTAACTCTGTAGAAGAACGGACAAGG |
| 16 | rbc71 | TAGCGGCCGCTTAGTGATGGTGATGGTGATGAACAT |
| | | TAGCTGTTGGAGTTTCTAC |
| 17 | rbc66 | TTGGTGGTGGTACAATTGGACATCCAGATGGAATTCA |
| | | AG |
| 18 | rbc67 | CTTGAATTCCATCTGGATGTCCAATTGTACCACCACC |
| | | AA |
| 19 | rbc64 | CAAAAATGGGATATTGGGACCCTAACTATGCAGTAAA |
| | | AG |
| 20 | rbc65 | CTTTTACTGCATAGTTAGGGTCCCAATATCCCATTTT |
| | | TG |
| 21 | rbc80 | TAGCGGCCGCTTAAGCATAATCTGGAACATC |
| 22 | rbc68 | CCGTGGCCAAGATTAACACAAGGAACTTTTTCTTTCC |
| | | TACC |
| 23 | rbc69 | TAGCGGCCGCTTAAGCATAATCTGGAACATCATATGG |
| | | ATAATATCTAGATCCTTCTGGCTTAT |
| 24 | Rbc134 | TTACCATGGCTAACTCTGTAGAAGAACGG |

SEQ ID NO: 25 is the DNA sequence of wildtype A. edulis rbcL gene (AErbcL)
SEQ ID NO: 26 is the DNA sequence of wildtype mature A. edulis RUBISCO small subunit coding region (AErbcS)
SEQ ID NO: 27 is the DNA sequence of A. edulis rbcL transgene designed for nuclear expression (nAErbcL).
SEQ ID NO: 28 is the DNA sequence of A. edulis rbcS transgene designed for nuclear expression (nAErbcS).
SEQ ID NO: 29 is the DNA sequence of A. edulis rbcL transgene designed for chloroplast expression (cpAErbcL).
SEQ ID NO: 30 is the DNA sequence of the nGMNrbcL transgene.
SEQ ID NO: 31 is the amino acid sequence of the nGMNrbcL transgene. Tomato transit peptide: Met1-Cys57. G. monilis RUBISCO LSU: Met58-Val549. 6-His tag: His550-His555.
SEQ ID NO: 32 is the DNA sequence of the nGMNrbcS transgene.
SEQ ID NO: 33 is the Amino acid sequence of the nGMNrbcS transgene. Tomato transit peptide: Met1-Cys57. Mature G. monilis RUBISCO SSU: Met58-Tyr199. HA tag: Tyr200-Ala208.
SEQ ID NO: 34 is the DNA sequence of cpGMNrbcL transgene
SEQ ID NO: 35 is the amino acid sequence of cpGMNrbcL transgene—G. monilis Rubisco LSU: Met1-Val488. 6-His tag: His489-His5104.
SEQ ID NO: 36 is the amino acid sequence for AErbcL
SEQ ID NO: 37 is the amino acid sequence for AErbcS
SEQ ID NO: 38 is the amino acid sequence for nAErbcL
SEQ ID NO: 39 is the amino acid sequence for nAErbcS
SEQ ID NO: 40 is the amino acid sequence for cpAErbcL
SEQ ID NO: 41 is the DNA sequence of Zea mays, rbcL (ribulose-1,5-bisphosphate carboxylase/oxygenase large subunit)
SEQ ID NO: 42 is the amino acid sequence of Zea mays, rbcL (ribulose-1,5-bisphosphate carboxylase/oxygenase large subunit)
SEQ ID NO: 43 is the DNA sequence of Zea mays, rbcS (ribulose-1,5-bisphosphate carboxylase/oxygenase small subunit)
SEQ ID NO: 44 is the amino acid sequence of Zea mays, rbcS (ribulose-1,5-bisphosphate carboxylase/oxygenase small subunit)
SEQ ID NO: 45 is the DNA sequence of Saccharum officinarum, rbcL (ribulose-1,5-bisphosphate carboxylase/oxygenase large subunit)
SEQ ID NO: 46 is the amino acid sequence of Saccharum officinarum, rbcL (ribulose-1,5-bisphosphate carboxylase/oxygenase large subunit)
SEQ ID NO: 47 is the DNA sequence of Saccharum officinarum, rbcS (ribulose-1,5-bisphosphate carboxylase/oxygenase small subunit)
SEQ ID NO: 48 is the amino acid sequence of Saccharum officinarum, rbcS (ribulose-1,5-bisphosphate carboxylase/oxygenase small subunit)
SEQ ID NO: 49 is the DNA sequence of Amaranthus hypochondriacus, rbcL (ribulose-1,5-bisphosphate carboxylase/oxygenase large subunit)
SEQ ID NO: 50 is the amino acid sequence of Amaranthus hypochondriacus, rbcL (ribulose-1,5-bisphosphate carboxylase/oxygenase large subunit)
SEQ ID NO: 51 is the DNA sequence of Amaranthus hypochondriacus, rbcS (ribulose-1,5-bisphosphate carboxylase/oxygenase small subunit)
SEQ ID NO: 52 is the amino acid sequence of Amaranthus hypochondriacus, rbcS(ribulose-1,5-bisphosphate carboxylase/oxygenase small subunit)
SEQ ID NO: 53 is the DNA sequence of Griffithsia monilis, rbcL (ribulose-1,5-bisphosphate carboxylase/oxygenase large subunit
SEQ ID NO: 54 is the amino acid sequence of Griffithsia monilis, rbcL (ribulose-1,5-bisphosphate carboxylase/oxygenase large subunit
SEQ ID NO: 55 is the DNA sequence of Griffithsia monilis, rbcS (ribulose-1,5-bisphosphate carboxylase/oxygenase small subunit)
SEQ ID NO: 56 is the amino acid sequence of Griffithsia monilis, rbcS (ribulose-1,5-bisphosphate carboxylase/oxygenase small subunit)

### DETAILED DESCRIPTION OF THE INVENTION

The invention relates to the expression of both the large and small subunits of either a C4 plant or Rhodophyte derived RUBISCO in a C3 plant. In particular, the A. edulis RUBISCO large subunit coding sequence and mature small subunit coding sequences were expressed in tobacco and soybean and the G. monilis RUBISCO large subunit coding sequence was expressed in tobacco. The invention provides a method for development of nucleus and chloroplast transformation vectors for functional expression of the A. edulis and G. monilis RUBISCO enzymes in tobacco and soybean, or other hosts to improve photosynthesis for better crop performance.

The ability to express the more efficient C4 plant or Rhodophyte-derived RUBISCO in a C3 plant is useful as such expression is expected to improve the growth rate and yield of the C3 plant. Many C3 plants are critical to the world food supply including soybean, rice, canola, and wheat.

The following definitions and abbreviations are to be used for the interpretation of the claims and the specification.

The term “RUBISCO” will mean the enzyme ribulose-1,5-bisphosphate carboxylase/oxygenase (Rubisco, E. C. 4.1.1.39), as more fully described below.

“Plant” refers to any higher or lower plant, particularly dicots and monocots. As used herein the term “C3 plant” means a plant that only uses the Calvin cycle to fix carbon dioxide, i.e., uses “C3 carbon fixation”. C3 carbon fixation is a process that converts carbon dioxide and ribulose bisphosphate into 3-phosphoglycerate in the first step of the Calvin cycle. The term “C4 plant” means a plant that initially fixes CO₂ using the enzyme phosphoenolpyruvate carboxylase. The CO₂ is later released from the resulting C4 acids and then refixed by RUBISCO C3 plants preferred for use in the present invention include, but are not limited to, tobacco, soybean, rice, canola, cotton and wheat. A preferred C4 plant from which the RUBISCO of the invention is derived is Amaranthus edulis.

“Rhodophytes” are a large group, about 5000-6000 of mostly multicellular, photosynthetic, marine algae, which occur in freshwater and/or soil habitats.

The term “L8S8 RUBISCO” refers to the hexadecameric form of the RUBISCO enzyme consisting of eight large subunits (each about 55 kD) and eight small subunits (each about 14 kD).

“rbcL” is the designation for the RUBISCO large subunit gene.

“rbcS” is the designation for the RUBISCO small subunit gene.

“LSU” is the abbreviation for the RUBISCO large subunit.

“SSU” is the abbreviation for the RUBISCO small subunit.

“Tobacco rbcL-knockout plant” or “tobacco rbcL-KO plant”, refers to a plant in which the naturally-occurring rbcL gene in the tobacco chloroplast genome is disrupted, leading to the functional inactivation of the endogenous RUBISCO. In this invention, the rbcL-KO tobacco was developed by Icon Genetics (Halle, Germany). In the chloroplast genome of this plant, the majority of the rbcL coding sequence was replaced with a green fluorescent protein (GFP) gene. The result created an rbcL fragment that encoded the N-terminal 59 amino acids translationally fused with the GFP gene. Thus, there is no functional rbcL gene in the genome and the plant has no LSU accumulation. In the absence of LSU, the SSU also does not accumulate, possibly because it is proteolytically degraded when not present in a holoenzyme complex with the LSU. Thus, in the rbcL-KO tobacco there is neither LSU nor SSU protein, no RUBISCO activity, and no photosynthesis activity. The homoplastomic rbcL knockout plant is pale and only survives when sugar is provided. The chimeric plant containing both WT and rbcL-KO sectors is able to grow slowly without sugar supplement since some sections of leaves have wild type chloroplast genomes, appear green, and carry out photosynthesis.

“Progeny” comprises any subsequent generation of a plant.

“Transgenic plant” includes reference to a plant which comprises within its genome a heterologous polynucleotide. Preferably, the heterologous polynucleotide is stably integrated within the genome such that the polynucleotide is passed on to successive generations. The heterologous polynucleotide may be integrated into the genome alone or as part of a recombinant DNA construct.

“Heterologous” with respect to sequence means a sequence that originates from a foreign species, or, if from the same species, is substantially modified from its native form in composition and/or genomic locus by deliberate human intervention.

“Transgenic” refers to any cell, cell line, callus, tissue, plant part or plant, the genome of which has been altered by the presence of a heterologous nucleic acid, such as a recombinant DNA construct, including those initial transgenic events as well as those created by sexual crosses or asexual propagation from the initial transgenic event. The term “transgenic” as used herein does not encompass the alteration of the genome (chromosomal or extra-chromosomal) by conventional plant breeding methods or by naturally occurring events such as random cross-fertilization, non-recombinant viral infection, non-recombinant bacterial transformation, non-recombinant transposition, or spontaneous mutation.

“Genome” as it applies to plant cells encompasses not only chromosomal DNA found within the nucleus, but organelle DNA found within subcellular components (e.g., mitochondrial, plastid) of the cell.

“Polynucleotide”, “nucleic acid sequence”, “nucleotide sequence”, or “nucleic acid fragment” are used interchangeably and is a polymer of ribonucleic acid (RNA) or deoxyribonucleic acid (DNA) that is single- or double-stranded, optionally containing synthetic, non-natural or altered nucleotide bases. Nucleotides (usually found in their 5′-monophosphate form) are referred to by their single letter designation as follows: “A” for adenylate or deoxyadenylate (for RNA or DNA, respectively), “C” for cytidylate or deoxycytidylate, “G” for guanylate or deoxyguanylate, “U” for uridylate, “T” for deoxythymidylate, “R” for purines (A or G), “Y” for pyrimidines (C or T), “K” for G or T, “H” for A or C or T, “I” for inosine, and “N” for any nucleotide.

“Polypeptide”, “peptide”, “amino acid sequence” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers.

“Messenger RNA (mRNA)” refers to the RNA that is without introns and that can be translated into protein by the cell.

“cDNA” refers to a DNA that is complementary to and synthesized from a mRNA template using the enzyme reverse transcriptase. The cDNA can be single-stranded or converted into the double-stranded form using the Klenow fragment of DNA polymerase I.

“Mature” protein refers to a post-translationally processed polypeptide; i.e., one from which any pre- or pro-peptides present in the primary translation product have been removed.

“Precursor” protein refers to the primary product of translation of mRNA; i.e., with pre- and pro-peptides still present. Pre- and pro-peptides may be and are not limited to intracellular localization signals.

“Isolated” refers to materials, such as nucleic acid molecules and/or proteins, which are substantially free or otherwise removed from components that normally accompany or interact with the materials in a naturally occurring environment. Isolated polynucleotides may be purified from a host cell in which they naturally occur. Conventional nucleic acid purification methods known to skilled artisans may be used to obtain isolated polynucleotides. The term also embraces recombinant polynucleotides and chemically synthesized polynucleotides.

“Recombinant” refers to an artificial combination of two otherwise separated segments of sequence, e.g., by chemical synthesis or by the manipulation of isolated segments of nucleic acids by genetic engineering techniques. “Recombinant” also includes reference to a cell or vector, that has been modified by the introduction of a heterologous nucleic acid or a cell derived from a cell so modified, but does not encompass the alteration of the cell or vector by naturally occurring events (e.g., spontaneous mutation, natural transformation/transduction/transposition) such as those occurring without deliberate human intervention.

“Recombinant DNA construct” refers to a combination of nucleic acid fragments that are not normally found together in nature. Accordingly, a recombinant DNA construct may comprise regulatory sequences and coding sequences that are derived from different sources, or regulatory sequences and coding sequences derived from the same source, but arranged in a manner different than that normally found in nature.

“Regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include, but are not limited to, promoters, translation leader sequences, introns, and polyadenylation recognition sequences.

“Promoter” refers to a nucleic acid fragment capable of controlling transcription of another nucleic acid fragment.

“Promoter functional in a plant” is a promoter capable of controlling transcription in plant cells whether or not its origin is from a plant cell. A promoter that is functional in a plant include those that are useful for expression in both the plant nucleus as well as the choloroplast.

“Tissue-specific promoter” and “tissue-preferred promoter” are used interchangeably, and refer to a promoter that is expressed predominantly but not necessarily exclusively in one tissue or organ, but that may also be expressed in one specific cell.

“Developmentally regulated promoter” refers to a promoter whose activity is determined by developmental events.

“Operably linked” refers to the association of nucleic acid fragments in a single fragment so that the function of one is regulated by the other. For example, a promoter is operably linked with a nucleic acid fragment when it is capable of regulating the transcription of that nucleic acid fragment.

“Expression” refers to the production of a functional product. For example, expression of a nucleic acid fragment may refer to transcription of the nucleic acid fragment (e.g., transcription resulting in mRNA or functional RNA) and/or translation of mRNA into a precursor or mature protein.

“Phenotype” means the detectable characteristics of a cell or organism.

“Introduced” in the context of inserting a nucleic acid fragment (e.g., a recombinant DNA construct) into a cell, means “transfection” or “transformation” or “transduction” and includes reference to the incorporation of a nucleic acid fragment into a eukaryotic or prokaryotic cell where the nucleic acid fragment may be incorporated into the genome of the cell (e.g., chromosome, plasmid, plastid or mitochondrial DNA), converted into an autonomous replicon, or transiently expressed (e.g., transfected mRNA).

A “transformed cell” is any cell into which a nucleic acid fragment (e.g., a recombinant DNA construct) has been introduced.

“Transformation” as used herein refers to both stable transformation and transient transformation.

“Stable transformation” refers to the introduction of a nucleic acid fragment into a genome of a host organism resulting in genetically stable inheritance. Once stably transformed, the nucleic acid fragment is stably integrated in the genome of the host organism and any subsequent generation.

“Transient transformation” refers to the introduction of a nucleic acid fragment into the nucleus, or DNA-containing organelle, of a host organism resulting in gene expression without genetically stable inheritance.

“Allele” is one of several alternative forms of a gene occupying a given locus on a chromosome. When the alleles present at a given locus on a pair of homologous chromosomes in a diploid plant are the same that plant is homozygous at that locus. If the alleles present at a given locus on a pair of homologous chromosomes in a diploid plant differ that plant is heterozygous at that locus. If a transgene is present on one of a pair of homologous chromosomes in a diploid plant that plant is hemizygous at that locus.

“Contig” refers to a nucleotide sequence that is assembled from two or more constituent nucleotide sequences that share common or overlapping regions of sequence homology. For example, the nucleotide sequences of two or more nucleic acid fragments can be compared and aligned in order to identify common or overlapping sequences. Where common or overlapping sequences exist between two or more nucleic acid fragments, the sequences (and thus their corresponding nucleic acid fragments) can be assembled into a single contiguous nucleotide sequence.

“Codon degeneracy” refers to divergence in the genetic code permitting variation of the nucleotide sequence without affecting the amino acid sequence of an encoded polypeptide. Accordingly, the instant invention relates to any nucleic acid fragment comprising a nucleotide sequence that encodes all or a substantial portion of the amino acid sequences set forth herein. The skilled artisan is well aware of the “codon-bias” exhibited by a specific host cell in usage of nucleotide codons to specify a given amino acid. Therefore, when synthesizing a nucleic acid fragment for improved expression in a host cell, it is desirable to design the nucleic acid fragment such that its frequency of codon usage approaches the frequency of preferred codon usage of the host cell.

“Synthetic nucleic acid fragments” can be assembled from oligonucleotide building blocks that are chemically synthesized using procedures known to those skilled in the art. These building blocks are ligated and annealed to form larger nucleic acid fragments which may then be enzymatically assembled to construct the entire desired nucleic acid fragment. “Chemically synthesized”, as related to a nucleic acid fragment, means that the component nucleotides were assembled in vitro. Manual chemical synthesis of nucleic acid fragments may be accomplished using well-established procedures, or automated chemical synthesis can be performed using one of a number of commercially available machines. Accordingly, the nucleic acid fragments can be tailored for optimal gene expression based on optimization of the nucleotide sequence to reflect the codon bias of the host cell. The skilled artisan appreciates the likelihood of successful gene expression if codon usage is biased towards those codons favored by the host. Determination of preferred codons can be based on a survey of genes derived from the host cell where sequence information is available.

The term “amplified” means the construction of multiple copies of a nucleic acid sequence or multiple copies complementary to the nucleic acid sequence using at least one of the nucleic acid sequences as a template. Amplification systems include the polymerase chain reaction (PCR) system, ligase chain reaction (LCR) system, nucleic acid sequence based amplification (NASBA, Cangene, Mississauga, Ontario), Q-Beta Replicase systems, transcription-based amplification system (TAS), and strand displacement amplification (SDA) (Diagnostic Molecular Microbiology: Principles and Applications, D. H. Persing et al., Ed., Am. Soc. Microbiol., Washington, D.C., 1993). The product of amplification is termed an amplicon.

The term “chromosomal location” includes reference to a length of a chromosome which may be measured by reference to the linear segment of DNA which it comprises. The chromosomal location can be defined by reference to two unique DNA sequences, i.e., markers.

“Plastid” refers to any of several pigmented or unpigmented cytoplasmic organelles such as chloroplasts, amyloplasts, leucoplasts, proplastids, and etioplasts, found in plant cells and other organisms, having various physiological functions, such as the synthesis and storage of food. All plastids are developmentally related to each other and all contain a plastome.

“Plastome” is the circular plastid genome of higher plants. It is approximately 150 kb in size and it encodes about 120 products.

The term “Transplastomic” means plants which have stably integrated into their plastome at least one expression cassette which is functional in plastids.

The term “Chloroplast” means a chlorophyll-containing plastid found in algal and green plant cells and includes all developmental stages of a chloroplast, such as proplastids, etioplasts, and mature chloroplasts. Chloroplasts and other plastids from all lower and higher plants are very similar in properties, and the present invention is therefore directed to all such organisms and their chloroplasts and plastids.

The term “Chaperonin” means protein complexes that assist the folding of nascent, native or non-native polypeptides into their fully-assembled, functional state.

The term “Primer” means a nucleic acid strand (or related molecule) that serves as a starting point for DNA replication.

“PCR” means polymerase chain reaction.

“Quantitative Polymerase chain reaction (qPCR) is a modification of the PCR used to rapidly measure the quantity of DNA, complementary DNA or RNA present in a sample.

“Oligo or oligonucleotide” refer to short sequences of nucleotides (RNA or DNA), typically with twenty or more bases

“Gene” or “genetic construct” refers to a nucleic acid fragment that expresses a specific protein, optionally including regulatory sequences preceding (5′ non-coding sequences) and following (3′ non-coding sequences) the coding sequence. “Native gene” or “wild type gene” refers to a gene as found in nature with its own regulatory sequences. “Chimeric gene” refers to any gene that is not a native gene, comprising regulatory and coding sequences that are not found together in nature. Accordingly, a chimeric gene may comprise regulatory sequences and coding sequences that are derived from different sources, or regulatory sequences and coding sequences derived from the same source, but arranged in a manner different than that found in nature. “Endogenous gene” refers to a native gene in its natural location in the genome of an organism. A “foreign” gene refers to a gene not normally found in the host organism, but that is introduced into the host organism by gene transfer. Foreign genes can comprise native genes inserted into a non-native organism, or chimeric genes. The term “open reading frame” refers to that portion of a gene or genetic construct that encodes a polypeptide but may be devoid of any regulatory elements.

A “terminator, or transcription terminator” is a section of genetic sequence that marks the end of gene or operon on genomic DNA for transcription.

“Polylinker region” means a DNA fragment on a vector/plasmid, containing multiple unique restriction enzyme recognition sites for other DNA fragments to be cloned/integrated into vector/plasmid conveniently.

“Foreign protein” means a heterologous protein.

“dNTP” is a mixture of dATP, dGTP, dCTP, and dTTP.

“GFP” means green fluorescent protein.

“NaEPPS buffer” is sodium [4-(2-hydroxyethyl)-1-piperazine-propanesulfonate buffer.

“rDNA” refers to Ribosomal Deoxyribonucleic Acid.

“Plasmid”, “vector” and “cassette” refer to an extra chromosomal element often carrying genes which are not part of the central metabolism of the cell, and usually in the form of circular double-stranded DNA fragments. Such elements may be autonomously replicating sequences, genome integrating sequences, phage or nucleotide sequences, linear or circular, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell.

“Transformation cassette” refers to a specific vector containing a foreign gene and having elements in addition to the foreign gene that facilitates transformation of a particular host cell. “Expression cassette” refers to a specific vector containing a foreign gene and having elements in addition to the foreign gene that allow for enhanced expression of that gene in a foreign host. In the practice of the present invention, foreign DNA is provided for transformation into a plant chloroplast. “Foreign” or “exogenous” DNA refers to any DNA which is not found within the tobacco chloroplast in nature or modified from a native one. Thus, foreign DNA can encompass a wide variety of DNA molecules. Particularly preferred are DNA molecules containing an expression cassette; i.e., a DNA construct comprising a coding sequence and appropriate control sequences (e.g., promoter and appropriately matched transcription termination sequence) to provide for the proper expression of the coding sequence in the chloroplast. Typically, the expression cassette is flanked by convenient restriction sites to facilitate cloning. In a preferred embodiment, the foreign DNA used for transformation comprises an expression cassette flanked by chloroplast DNA to facilitate the stable integration of the expression cassette into the chloroplast genome by homologous recombination.

“Homologous targeting sequences” or “homology arms” are fragments of chloroplast genome sequences, flanking a chimeric transgene structure in a plasmid. They function to exchange the chimeric transgene structure into the chloroplast genome to replace genomic sequence between the homologous targeting sequences through homologous recombination. Homologous targeting sequence on one side is left targeting region (LTR) and on other side is right targeting region (RTR).

“Homologous recombination” (or general recombination) is defined as the exchange of homologous segments anywhere along a length of two DNA molecules. An essential feature of general recombination is that the enzymes responsible for the recombination event can presumably use any pair of homologous sequences as substrates, although some types of sequence may be favored over others.

The term “sequence analysis software” refers to any computer algorithm or software program that is useful for the analysis of nucleotide or amino acid sequences. Sequence alignments and percent identity calculations may be determined using a variety of comparison methods designed to detect homologous sequences including, but not limited to, the Megalign® program of the LASERGENE® bioinformatics computing suite (DNASTAR® Inc., Madison, Wis.). Unless stated otherwise, multiple alignment of the sequences provided herein were performed using the Clustal V method of alignment (Higgins and Sharp, CABIOS. 5, 151-153, 1989) with the default parameters (GAP PENALTY=10, GAP LENGTH PENALTY=10). Default parameters for pairwise alignments and calculation of percent identity of protein sequences using the Clustal V method are KTUPLE=1, GAP PENALTY=3, WINDOW=5 and DIAGONALS SAVED=5. For nucleic acids these parameters are KTUPLE=2, GAP PENALTY=5, WINDOW=4 and DIAGONALS SAVED=4. After alignment of the sequences, using the Clustal V program, it is possible to obtain “percent identity” and “divergence” values by viewing the “sequence distances” table on the same program; unless stated otherwise, percent identities and divergences provided and claimed herein were calculated in this manner. Unless otherwise stated, “BLAST” sequence identity/similarity values provided herein refer to the value obtained using the BLAST 2.0 suite of programs using default parameters (Altschul, et al., J. Mol. Biol. 215, 403-410, 1990). Software for performing BLAST analyses is publicly available, e.g., through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always>0) and N (penalty score for mismatching residues; always<0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix (Henikoff, S., and Henikoff, J. G., Proc. Natl. Acad. Sci. USA 89, 10915-10919, 1989).

The term “holoenzyme” in this context means an active, complex enzyme consisting of all subunits.

The terms “k_{cat}” and “Kₘ” are known to those skilled in the art and are described in Enzyme Structure and Mechanism, 2^{nd} ed. (Ferst; W.H. Freeman: NY, pp 98-120, 1985). The term “k_{cat}”, often called the “turnover number”, is defined as the maximum number of substrate molecules converted to products per active site per unit time, or the number of times the enzyme turns over per unit time. k_{cat}=Vmax/[E], where [E] is the enzyme concentration (Ferst, supra). The terms “total turnover” and “total turnover number” are used herein to refer to the amount of product formed by the reaction of a RUBISCO enzyme with substrate.

The term “specific activity” means enzyme units/mg protein where an enzyme unit is defined as moles of product formed/minute under specified conditions of temperature, pH, [S], etc.

RUBISCO “specificity”, sometimes designated as Tau or S_{c/o}, is a measure of the rates of carboxylation to oxygenation at equal concentrations of CO₂ and O₂. It is defined by the expression:
(k^{c}_{cat}×Kₒ)/(k^{o}_{cat}×K_{c}) where:
k^{c}_{cat} is the turnover number for carboxylation,
k^{o}_{cat} is the turnover number for oxygenation
K_{c} is the Michaelis constant for CO₂
Kₒ is the Michaelis constant for O₂

Standard recombinant DNA and molecular cloning techniques used herein are well known in the art and are described more fully in Sambrook, J., Fritsch, E. F. and Maniatis, T. Molecular Cloning: A Laboratory Manual; Cold Spring Harbor Laboratory Press: Cold Spring Harbor, 1989 (hereinafter “Sambrook”).

The invention relates to the effective and high level expression of either C4 plant derived or Rhodophyte RUBISCO enzymes in C3 plants. Specifically the invention provides a method for the recombinant expression of an L8S8 RUBISCO enzyme in a plant cell comprising:
a) providing a C3 plant cell comprising a transformation vector wherein the vector comprises a heterologous genetic construct encoding a plant protein selected from the group consisting of: the small subunit of a L8S8 RUBISCO enzyme and the large subunit of an L8S8 RUBISCO enzyme, wherein the large and small subunits of the L8S8 RUBISCO enzyme are derived from a C4 plant or a rhodophyte; and
b) growing the plant cell under conditions whereby the protein is expressed in soluble form.

Sources of the appropriate forms of the RUBISCO enzymes as well as preferred expression hosts and methods of transforming these hosts for effected RUBISCO expression will be described below in detail.

### RUBISCO Enzyme

Three major forms of RUBISCO enzymes are found in living organisms (Andrews T. J., & Lorimer, G. H., The Biochemistry of Plants, volume 10, 131-218, 1987 and Miziorko, H. M., & Lorimer, G. H., Annu. Rev. Biochem., 52, 507-535, 1983). Form I, which is found in higher plants, algae and most other photosynthetic organisms, is a complex molecule consisting of eight large (L, Mr=55, 000) and eight small (S, Mr=14,000) subunits, forming an L₈S₈ complex. In higher plants, the large subunit (LSU) is encoded by the chloroplast gene rbcL while the small subunit (SSU) is encoded by the nuclear gene rbcS. After synthesis, SSU is translocated from the cytosol to the chloroplast, processed to remove the transit peptide, and assembled with the LSU (Spreitzer, R. J. and Salvucci, M. E., Annu. Rev. Plant Biol. 53, 449-475, 2002). On the other hand, form II, which is primarily found in certain bacteria, e.g., the photosynthetic bacterium Rhodospirillum rubrum (R. rubrum), is a dimer of large subunits, L₂, (Tabita, F. R. and McFadden, B, A., Arch. Microbiol., 99, 231-240, 1974) that differ substantially in sequence from form I large subunits. Depending on the source, form II may be oligomerized to form dimmers, tetramers, or even larger oligomers (Li, H., et al., Structure, 13, 779-789, 2005). Form III also contains only a LSU and forms dimers (L₂) or decamers [(L₂)₅] (Li, H., supra). In all forms the L subunit carries the catalytic function of the enzyme.

The RUBISCO enzyme, especially the one in C3 plants such as tobacco and soybean, is a sub-optimal enzyme in 2 respects. First, its catalytic activity (k^{c}_{cat}˜3s⁻¹), is relatively slow for an enzyme that performs such a high flux reaction in photosynthetic carbon fixation. To compensate for its low activity, plants accumulate large amounts of RUBISCO enzyme in their green tissues. Indeed, RUBISCO accounts for about half of the leaf's total soluble proteins. Increasing RUBISCO'S catalytic rate, therefore, would reduce commensurately the requirement for this massive accumulation of enzyme and allow the plant to reapportion those resources to other functions. Second, RUBISCO has poor ability to discriminate between CO₂ and O₂, leading to the catalysis of both carboxylation and oxygenation of RuBP. The ability of RUBISCO to discriminate between CO₂ and O₂ is measured by the specificity (also termed Tao, τ, or S_{c/o}). The specificity represents the ratio of the rates of carboxylation to oxygenation in equal concentrations of CO₂ and O₂ and is given by the expression [(k^{c}_{cat})(Kₒ)]/[(k^{o}_{cat})(K_{c})] (Laing et al., Plant Physiol., 54, 678-685, 1974). The RUBISCO from plants typically has a specificity of 80-100, however, because of the much higher concentration of O₂ than CO₂ in the atmosphere, about 25% of the turnovers of RUBISCO in most plants are oxygenations.

Amaranthus edulis is a dicot C4 plant and its RUBISCO has better kinetic properties (e.g., k^{c}_{cat} of 7.3 and Tao of 82 (Seemann, J. R., et al., Plant Physiol., 74, 791-794, 1984 and Zhu. X. -G., et al. (supra)) compared to major C3 crops such as soybean and tobacco. Therefore, it is possible to increase the efficiency of photosynthesis in C3 plants by expressing A. edulis RUBISCO in them. Based on photosynthetic modeling, complete replacement of the endogenous RUBISCO with the A. edulis RUBISCO could increase photosynthesis by 17% in an average C3 plant (Zhu. X. -G., et al., (supra)). Neither the A. edulis rbcL nor rbcS genes have been previously cloned nor expressed in any plant. However, rbcL has been cloned from two other species of Amaranthus. The rbcL gene of A. hypochondriacus has an accession number of X51964 (Michalowski, C. B. et al., Nucleic Acids Res., 18, 2187, 1990.). The rbcL gene of A. tricolor has an accession number of X53980 (Rettig, J. H., et al., Taxon 41, 201-209, 1992). On the other hand, three rbcS genes have been cloned from A. hypochondriacus, (accession #AF150665, AF150666, and AF150667) (Corey, A. C., et al., Plant Physiol., 120, 934, 1999). Foreign higher plant RUBISCOS have been expressed in tobacco through nuclear and chloroplast transformation approaches. For example, a sunflower (Helianthus annuus) rbcL gene has been introduced into the tobacco chloroplast genome between atpB and accD. The transformant produced functional sunflower RUBISCO LSU up to approximately 15% total soluble protein (Kanevski, I., et al., Plant Physiol. 119,133-142, 1999). In another case, a tobacco chloroplast rbcL gene was relocated to the tobacco nuclear genome and expressed in the cytosol. With the help of a pea RUBISCO SSU transit peptide, the LSU accumulated and functioned in the chloroplast (Kanevski, I. and Maliga, P., Proc. Natl. Acad. Sci. USA 91, 1969-1973, 1994). Similar experiments have not been reported in soybean. Accordingly then, it is within the scope of the invention to provide method for the recombinant expression of RUBISCO large and small subunits of an L8S8 RUBISCO enzyme in a C3 plant cell where the RUBISCO enzymes is derived from a variety of C4 plants including, but not limited to corn (Zea mays,) and sugar cane (Saccharum officinarum). The sequences of the large and small subunits of the RUBISCO enzyme for some typical C4 plants are listed herein as SEQ ID NO:'s 41-52.

Griffithsia monilis is a Rhodophyte which also has a RUBISCO enzyme with much improved kinetic properties, k^{c}_{cat} of 2.6, tau of 167 (Whitney S. M., et al., Plant J., 26, 535-547, 2001) relative to higher plants. Based on these kinetic properties, modeling indicates that if the endogenous RUBISCO in C3 plants could be replaced with a functional version of this enzyme, then the photosynthesis of the plant would be improved by 27% (Zhu. et al., supra). However, previous attempts to express algal L8S8 RUBISCO genes in plants have been unsuccessful, producing only insoluble, inactive protein (Whitney, S. M., et al., Plant J., 26, 535-547, 2001).

In Griffithsia monilis rbcL and rbcS are both located in the plastid genome and form a gene cluster. Gene sequences and a PGEM plasmid containing both genes (pGm-rbcLS-TVE) were obtained from Australian National University. A comparison of the G. monilis rbcL and rbcS sequences with other rbcL and rbcS genes indicated that, at the amino acid level, G. monilis LSU has strong homology to the higher plant LSU (55% identity to LSU of corn, soybean, and tobacco). The G. monilis LSU was almost identical to LSU's from nine other members of the Griffithsia genus deposited in Genebank, having 95-97% identity at the amino acid level and 86-93% identity at the nucleotide level. On the other hand, G. monilis SSU has little homology to the higher plant SSU (<18% identity to the SSU of corn, soybean, and tobacco at the amino acid level). However, it has significant homology to Porphyridium aerugineum SSU, the only red algal SSU available in the public sequence database, at both the amino acid level (71% identity) and nucleotide level (69%). Although G. monilis rbcL and rbcS are plastid genes, their codon usage matches closely that of dicot nuclear genes. Thus, they can be used for tobacco and soy nuclear transformation without optimization.

In this invention, A. edulis rbcL and rbcS, which had not been previously cloned, were cloned and inserted into chimeric transgenes for tobacco nuclear and chloroplast expression, as well as soybean nuclear expression. G. monilis rbcL and rbcS were also expressed with the same approaches. Heterologous RUBISCO proteins expressed using different approaches were characterized in detail.

Within the context of the present invention L8S8 RUBISCO large subunit proteins that are expected to be expressed in C3 plants in soluble form are those that are at least about 90% identical to the amino acid sequences as set forth in SEQ ID NO:36, 38 and 40 (Amaranthus RUBISCO large subunit, including modifications for nuclear and chloroplast expression) and SEQ ID NO:s 31 and 35 (Griffithsia RUBISCO large subunit, including modifications for nuclear and chloroplast expression) over the full length of the protein sequence using the Clustal V method of alignment (described by Higgins and Sharp, CABIOS, 5:151-153 (1989); Higgins, D. G. et al., Comput. Appl. Biosci., 8:189-191(1992); found in the MegAlign™ v6.1 program of the LASERGENE bioinformatics computing suite (DNASTAR Inc.)). More preferred amino acid sequences corresponding to RUBISCO large subunits are at least about 95% identical to SEQ ID NO:36, 38 and 40 and SEQ ID NO:s 31 and 35. Alternatively, nucleic acid sequences encoding RUBISCO large subunit proteins useful for expression in C3 plants may be determined according to stringent hybridization conditions (0.1×SSC, 0.1% SDS, 65° C. and washed with 2×SSC, 0.1% SDS followed by 0.1×SSC, 0.1% SDS) where those nucleic acid sequences that hybridize under these conditions to any nucleic acid sequence encoding the specific large subunit proteins described herein, including, but not limited to SEQ ID NO:s 25, 27 and 29 (Amaranthus RUBISCO large subunit, including modifications for nuclear and chloroplast expression) and SEQ ID NO: 30 and 34 (Griffithsia large RUBISCO subunit, including modifications for nuclear and chloroplast expression) will be expressed in soluble form.

Similarly L8S8 RUBISCO small subunit proteins that are expected to be expressed in C3 plants in soluble form are those that are at least about 90% identical to the amino acid sequences as set forth in SEQ ID NO:37 and 39 (Amaranthus RUBISCO small subunit, including modifications for nuclear and chloroplast expression) and SEQ ID NO:s 33 (Griffithsia RUBISCO small subunit, including modifications for nuclear and chloroplast expression) over the full length of the protein sequence using the Clustal V method of alignment (described by Higgins and Sharp, CABIOS, 5:151-153 (1989); Higgins, D. G. et al., Comput. Appl. Biosci., 8:189-191(1992); found in the MegAlign™ v6.1 program of the LASERGENE bioinformatics computing suite (DNASTAR Inc.)). More preferred amino acid sequences corresponding to RUBISCO small subunits are at least about 95% identical to SEQ ID NO:37 and 39 and SEQ ID NO:33. Alternatively nucleic acid sequences encoding RUBISCO small subunit proteins useful for expression in C3 plants may be determined according to stringent hybridization conditions (0.1×SSC, 0.1% SDS, 65° C. and washed with 2×SSC, 0.1% SDS followed by 0.1×SSC, 0.1% SDS ) where those nucleic acid sequences that hybridize under these conditions to any nucleic acid sequence encoding the specific small subunit proteins described herein, including, but not limited to SEQ ID NO:s 26 and 28 (Amaranthus RUBISCO small subunit, including modifications for nuclear and chloroplast expression) and SEQ ID NO: 32 (Griffithsia RUBISCO small subunit, including modifications for nuclear and chloroplast expression) will be expressed in soluble form.

### Plant Transformation

In this invention both nuclear and chloroplast transformations of plant cells were used.

Transgenic plant cells are placed in an appropriate selective medium for selection of transgenic cells that are then grown to callus. Shoots are grown from callus and plantlets generated from the shoot by growing in rooting medium. Various constructs normally will be joined to a marker for selection in plant cells. Conveniently, the marker may be resistance to a biocide (particularly an antibiotic such as kanamycin, G418, bleomycin, hygromycin, chloramphenicol, herbicide, spectinomycin or the like). The particular marker used will allow for selection of transformed cells as compared to cells lacking the DNA that has been introduced. Components of DNA constructs including transcription cassettes of this invention may be prepared from sequences which are native (endogenous) or foreign (exogenous) to the host. By “foreign” it is meant that the sequence is not found in the wild-type host into which the construct is introduced. Heterologous constructs will contain at least one region that is not native to the gene from which the transcription-initiation-region is derived.

To confirm the presence of the transgenes in transgenic cells and plants, a Southern blot or PCR analysis can be performed using methods known to those skilled in the art. Expression products of the transgenes can be detected in any of a variety of ways, depending upon the nature of the product (e.g., Western blot and enzyme assay). Once transgenic plants have been obtained, they may be grown to produce plant tissues or parts having the desired phenotype. The plant tissue or plant parts may be harvested, and/or the seed collected. The seed may serve as a source for growing additional plants with tissues or parts having the desired characteristics.

### Chloroplast Transformation

A desirable approach for expressing foreign RUBISCO genes is to introduce the gene into the genome of the chloroplast, the organelle in which the rbcL gene normally resides. Methods have been disclosed for introducing genes into the chloroplast genome. Chloroplast transformation vectors use regulatory and untranslated regions (promoters, ribosome binding sites and terminators) of chloroplast, bacterial or viral origin to control expression of marker genes and specific genes of interest (such as RUBISCO genes). Promoters that have been used for chloroplast transformation include psbA, Prrn, rbcL, Ptrc. 5′ untranslated regions that have been used include the ribosome binding site from gene10 of bacteriophage T7 and the RBS from the rbcL or atpB genes (Daniell, H., et al, Trends in Biotech., 23:238-245, 2005). 3′ untranslated regions that have been used include sequences from psbA, rps16, rbcL and rrn (Daniell, H., et al, Trends in Biotech., 23:238-245, 2005). These genes are flanked by chloroplast sequences (homologous targeting sequences) that are homologous to specific sites in the chloroplast genome. Appropriate left and right homology regions can be identified from the available chloroplast genomic sequences from various species that are provided in public databases. Generally, these homology regions are each about 0.5 to 1.5 kb in length. Standard PCR techniques can be used to amplify these sequences from genomic DNA and then clone them into the chloroplast transformation vector such that they flank the transformation cassette. A number of integration sites have been used to introduce transgenes into the chloroplast genome. These include the regions between to following pairs of loci: trnI/trnA, trnV/rps12/7, rbcL/accD, trnH/pbA, trnG/trnfM, ycf3/trnS, petA/psbJ, 5′rps12/cIpP, petD/rpoA, ndhB/rps7, 3′rps12/trnV, rrn16/trnI, trnN/trnR, rpl32/trnL (Maliga, P, Annu. Rev. Plant Biol., 55:289-313, 2004).

Following delivery of the chloroplast transformation vector into a chloroplast, these homologous targeting sequences mediate homologous recombination between the introduced vector and the chloroplast genome, resulting in the insertion of the sequence in the vector located between the homologous targeting sequences into the chloroplast genome. The introduced genes are then expressed at very high levels. Methods disclosed for plastid transformation in higher plants include the particle gun delivery of DNA containing a selectable marker and targeting of the DNA to the plastid genome through homologous recombination (Svab, Z. et al., Proc. Natl. Acad. Sci., USA, 87, 8526-8530, 1990 and Svab, Z. and Maliga, P. Proc. Natl. Acad. Sci., USA, 90, 913-917, 1993 and Staub, J. M. and Maliga, P. EMBO J., 12, 601-606, 1993 and U.S. Pat. Nos. 5,451,513 and 5,545,818). For some species, protoplasts can also be utilized for chloroplast transformation (O'Neill, C., et al., Plant J., 3, 729-38; 1993 and Spoerlein, B., et al., Theor. Appl. Gen., 82, 717-22, 1991).

Following introduction of the chloroplast transformation vectors, the treated cultures are placed on tissue culture medium containing the appropriate selection agent. The most commonly used selection marker is the aadA gene coding for streptomycin/ spectinomycin adenyltransferase (Svab, Z. et al., Proc. Natl. Acad. Sci., USA, 90, 913-917, 1993). Genes conferring resistance to kanamycin (NPTII or AphA6) have also been used (Carrer, H., et al., Mol. Gen. Genetics, 241, 49-56, 1993 and Huang, F. -C.; et al., Mol. Gen. Genomics, 268, 19-27, 2002). After a suitable period of incubation on selection medium, transformed cell lines can be identified and grown to a stage that allows regeneration of whole plants. The regeneration processes are basically identical to those used for standard nuclear transformation events. Special care must be taken to ensure that selection and regeneration conditions promote the elimination of all wild-type chloroplast genomes. The status of the proportion of wild-type to transformed chloroplast genomes can be monitored by standard molecular techniques including Southern and PCR analysis.

Chloroplast transformation has been accomplished in a number of species including tobacco, soybean, rice, potato, soybean, duckweed, lettuce, cabbage, tomato, cotton, and poplar (Li, Yi-Nu et al., Zhongguo Nongye Kexue, Beijing, China, 40, 1849-1851, 2007, and Hou, Bingkai; et al., 28, 187-192, 2002, and Nguyen, T., et al, Plant Sci., 168, 1495-1500, 2005, and Dufourmantel, N., et al., Plant Mol. Biol., 55, 479-489, 2004, and Cox, K. M., and Peele, C. G. PCT lnt. Appl., 2005, WO 2005005643 A2 20050120, Kanamoto, H., et al., Transgenic Res., 15, 205-217, 2006, and Liu, Cheng-Wei, et al., Plant Cell Rep., 26, 1733-1744, 2007, and Wurbs, D., et al., Plant J., 49, 276-288; 2007, and Kumar, S., et al., Plant Mol. Biol., 56, 203-216, 2004, and Okumura, S., et al., Transgenic Res., 15, 637-646, 2006 and Svab, Z., et al., Proc. Natl. Acad. Sci. USA, 87, 8526-8530, 1990 and WO2004053133A1 and US20070039075A1 ).

Chloroplast transformation has been used to introduce foreign RUBISCO genes into the chloroplast genome. For example, the rbcL gene from sunflower (Kanevski, I., et al., supra) and the rbcM gene from the bacterium R. rubrum (Whitney, S. M. and Andrews, T. J. Plant Physiol., 133, 287-294, 2003 and Whitney, S. M. and Andrews, T. J., Proc. Natl. Acad. Sci., USA, 98, 14738-14743, 2001) were introduced into the chloroplast genome of tobacco. The rbcL and rbcS genes from the red alga Galdieria sulphuraria and the diatom Phaeodactylum tricornutum were also introduced into the chloroplast genome of tobacco (Whitney, S. M., et al., Plant J., 26, 535-547, 2001). Large amounts of RUBISCO protein was expressed from all these transgenes, however those from the red alga and the diatom were not properly assembled into a functional holoenzyme.

The psbA promoter from the plant chloroplast has been used in a number of studies to express foreign proteins to high levels. For example, Hayashi and coworkers (Plant Cell Physiol 44, 334-341, 2003) utilized the psbA promoter to drive expression of the green fluorescent protein in tobacco chloroplasts. The vector used the following elements where T before a component designates a terminator and P designates a promoter: TpsbA::aadA::Prrn//psbA::gfp::rps16. This construct was introduced between trnV and rps12/7 genes of the chloroplast genome. Staub and coworkers (Nature Biotechnol., 18, 333-338, 2000) used the psbA promoter to express human somatotropin (hST). They used the following vector with the components shown here: Trps16::aadA::Prrn//Trps16::hST::PpsbA. The transgenes were inserted between the trnV and rps12/7 genes of the chloroplast genome. Production of hST was 7% of total soluble protein. Dhingra and coworkers (Proc. Natl. Acad. Sci., USA, 101, 6315-6320, 2004) used the psbA promoter to control expression of the tobacco rbcS gene. The components of the vector were:
Prrn::aadA::term/PpsbA::rbcS::term. This construct was inserted between the trnl and trnA genes and gave 106% of the wild type rbcS levels.
Dufourmantel and coworkers (Plant Biotechnol. J., 5, 118-133, 2007) used the psbA promoter to drive expression of HPPD (4-hydroxyphenylpyruvate dioxygenase). The construct was inserted between the rbcL and accD genes in tobacco and lead to the accumulation of the foreign protein at 5% TSP. The structure of the vector that was used was:
PpsbA::HPPD::TrbcL//Prrn::aadA::TpsbA.

The psbA promoter and a host of other promoters, 5′ UTRs, terminators and homology regions have been used to express a wide range of proteins. These applications are summarized in the following review articles: (Daniell, H., et al., Transgenic Plants, 83-110, 2003 and Bock, R., Cur. Op. Biotechnol., 18, 100-106, 2007 and Grevich, J. and Daniell, H., Crit. Rev. Plant Sci, 24, 83-107, 2005 and Maliga, P., Ann. Rev. Plant Biol., 55, 289-313, 2004).

The gene encoding the RUBISCO large subunit, rbcL, is located in the chloroplast genome of higher plants. Using nuclear regulatory signals and a chloroplast targeting sequence an rbcL gene has been relocated to the nucleus for its functional expression (Kanevski , I. and Maliga, P. Proc. Natl. Acad. Sci., USA, 91, 1969-1973, 1994). However, while the rbcL gene is usually expressed at very high levels in the native cells, when it is expressed in the nucleus only about 3% of normal RUBISCO activity is observed. Thus, it is likely that successful expression of a foreign rbcL gene at high enough levels to completely support photosynthesis, would require, in addition to nuclear expression, insertion of the gene into the chloroplast genome. Moreover, it is likely that the endogenous rbcL gene will need to be eliminated from any potential crop with an improved Rubisco. This will likely be necessary in order to avoid deleterious interactions between the endogenous and foreign RUBISCO as well as to eliminate the production of the endogenous rbcL that is expensive to the plant in terms of nitrogen and energy.

### Nuclear Transformation

The development or regeneration of plants containing the foreign, exogenous gene that encodes a protein of interest in nuclei is well known in the art. Genes can routinely be introduced into the nuclear genome by an array of technologies. A transgenic plant of the present invention containing a desired polypeptide is cultivated using techniques well known to one skilled in the art. These techniques include transformation with DNA employing A. tumefaciens or A. rhizogenes as the transforming agent, electroporation, particle acceleration, etc. (EP 295959 and EP 138341). It is particularly preferred to use the binary type vectors of Ti and Ri plasmids of Agrobacterium spp. Ti-derived vectors transform a wide variety of higher plants, including monocotyledonous and dicotyledonous plants, such as soybean, cotton, rape, tobacco, and rice (Facciotti et al., Bio/Technology 3, 241-246, 1985, and Byrne et al., Plant Cell, Tissue and Organ Culture 8, 3-15, 1987, and Sukhapinda et al., Plant Mol. Biol. 8, 209-216, 1987 and Lorz et al., Mol. Gen. Genet. 199, 178-182, 1985, and Potrykus, Mol. Gen. Genet. 199, 183-188, 1985, Park et al., J. Plant Biol. 38, 365-71, 1995, and Hiei et al., Plant J. 6, 271-282, 1994). The use of T-DNA to transform plant cells has received extensive study and is amply described (EP 120516 and Hoekema, A., In: The Binary Plant Vector System, Offset-drukkerij Kanters B. V.; Alblasserdam,1985, Chapter V, Knauf, et al., Genetic Analysis of Host Range Expression by Agrobacterium (Molecular Genetics of the Bacteria-Plant Interaction, Puhler, A. Ed., Springer-Verlag: New York, 1983, pp, 245 and An et al., EMBO J. 4, 277-284, 1985). Other transformation methods are available to those skilled in the art, such as direct uptake of foreign DNA constructs (EP 295959), techniques of electroporation (Fromm et al. Nature (London) 319, 791, 1986) or high-velocity ballistic bombardment with metal particles coated with the nucleic acid constructs (Klein et al. Nature (London) 327, 70, 1987, and U.S. Pat. No. 4,945,050). Other vector systems suitable for introducing transforming DNA into a host plant cell include but are not limited to binary artificial chromosome (BIBAC) vectors (Hamilton, C. M., et al., Gene, 200, 107-116, 1997); and transfection with RNA viral vectors (Della-Cioppa, G., et al., Ann. N.Y. Acad. Sci., 792 (Engineering Plants for Commercial Products and Applications, 57-61, 1996). Additional vector systems also include plant selectable YAC vectors, such as those described by Mullen, J., et al., (Mol. Breeding, 4, 449-457, 1988).

Technology for introduction of DNA into cells is well known by one of skill in the art. Four general methods for delivering a gene into cells have been described: (1) chemical methods (Graham F. L., and van der Eb, A. J. Virology, 54, 536-539, 1973); (2) physical methods, such as microinjection (Capecchi, M., Cell, 22, 479-488, 1980), electroporation (Wong T. K., Neumann, E. Biochem. Biophys. Res. Commun., 107, 584-587, 1982; Fromm, M., et al., Proc. Natl. Acad. Sci. (USA), 82, 5824-5828, 1985; U.S. Pat. No. 5,384,253), the gene gun (Johnston, S. A., Tang, D. C., Methods Cell Biol., 43, 353-365, 1994), and vacuum infiltration (Bechtold N., et al., C.R. Acad. Sci. Paris, Life Sci., 316, 1194-1199, 1993); (3) viral vectors (Clapp, Clin. Perinatol., 20, 155-168, 1993; Lu, L., et al., J. Exp. Med., 178, 2089-2096, 1993; Eglitis M. A., Anderson, W. F., Biotechniques, 6, 608-614, 1988); and (4) receptor-mediated mechanisms (Curiel D. T., Hum. Gen. Ther., 3, 147-154, 1992; Wagner, E., et al., Proc. Natl. Acad. Sci., USA, 89, 6099-6103, 1992).

Acceleration methods that may be used include, for example, microprojectile bombardment and the like. One example of a method for delivering transforming nucleic acid molecules into plant cells is microprojectile bombardment. This method has been reviewed by Yang, N. S., and Christou, P. (eds.), Particle Bombardment Technology for Gene Transfer, Oxford Press, Oxford, England (1994). Non-biological particles (microprojectiles) may be coated with nucleic acids and delivered into cells by a propelling force. Exemplary particles include those comprised of tungsten, gold, platinum, and the like.

A particular advantage of microprojectile bombardment, in addition to it being an effective means of reproducibly transforming monocots, is that neither the isolation of protoplasts (Christou P., et al., Plant Physiol., 87, 671-674, 1988) nor the susceptibility to Agrobacterium infection is required. A particle delivery system suitable for use with the present invention is the helium acceleration PDS-1000/He gun, which is available from Bio-Rad Laboratories (Bio-Rad, Hercules, Calif.) (Sanford, J. C., et al., Technique, 3:3-16, 1991).

For the bombardment, cells in suspension may be concentrated on filters. Filters containing the cells to be bombarded are positioned at an appropriate distance below the microprojectile stopping plate. If desired, one or more screens are also positioned between the gun and the cells to be bombarded.

Alternatively, immature embryos or other target cells may be arranged on solid culture medium. The cells to be bombarded are positioned at an appropriate distance below the macroprojectile stopping plate or screen. Through the use of techniques set forth herein one may obtain 1000 or more loci of cells transiently expressing a marker gene. In bombardment transformation, one may optimize the pre-bombardment culturing conditions and the bombardment parameters to yield the maximum numbers of stable transformants. Both the physical and biological parameters for bombardment are important in this technology. Physical factors are those that involve manipulating the DNA/microprojectile precipitate or those that affect the flight and velocity of the microprojectiles. Biological factors include all steps involved in manipulation of cells before and immediately after bombardment, the osmotic adjustment of target cells to help alleviate the trauma associated with bombardment and, also, the nature of the transforming DNA, such as linearized DNA or intact supercoiled plasmids. It is believed that pre-bombardment manipulations are especially important for successful transformation of immature embryos.

Accordingly, it is contemplated that one may wish to adjust various aspects of the bombardment parameters in small scale studies to fully optimize the conditions. One may particularly wish to adjust physical parameters such as gap distance, flight distance, tissue distance, and helium pressure. One may also minimize the trauma of bombardment by modifying conditions that influence the physiological state of the recipient cells and which may therefore influence transformation and integration efficiencies. For example, the osmotic state, tissue hydration, and the subculture stage or cell cycle of the recipient cells may be adjusted for optimum transformation. The execution of other routine adjustments will be known by one of skill in the art in light of the present disclosure.

Alternatively, transformation of plant protoplasts can be achieved using methods based on calcium phosphate precipitation, polyethylene glycol treatment, electroporation, and combinations of these treatments (Potrykus, I., et al., Mol. Gen. Genet., 205, 193-200, 1986; Lorz, H., et al., Mol. Gen. Genet., 199, 178-182, 1985; Fromm , M., et al., Nature, 319, 791-793, 1986; Uchimiya, H., et al., Mol. Gen. Genet., 204, 204-207, 1986; Marcotte W. R., et al., Nature, 335, 454-457, 1988).

Once transformed, the cells can be regenerated by those skilled in the art. Of particular relevance are the recently described methods to transform foreign genes into commercially important crops, such as rapeseed (De Block, M., et al., Plant Physiol. 91:694-701, 1989), sunflower (Everett, N. P., et al., Bio/Technology 5, 1201-1204, 1987), soybean (McCabe, D. E., et al., Bio/Technology 6, 923-926, 1988; Hinchee, M. A. W., et al., Bio/Technology 6, 915-922, 1988; Chee, P. P., et al., Plant Physiol., 91, 1212-1218, 1989; Christou, P., et al., Proc. Natl. Acad. Sci USA, 86, 7500-7504, 1989); EP 301749), rice (Hiei, Y., et al., Plant J. 6, 271-282, 1994), and corn (Gordon-Kamm, W. J., et al., Plant Cell 2, 603-618, 1990 and Fromm, M. E., et al., Biotechnology 8, 833-839, 1990).

### RUBISCO Enzyme Assay

To demonstrate RUBISCO activity in the transplastomic plants, crude protein extracts and purified RUBISCO complexes were dialyzed against a solution containing 0.1 M NaEPPS pH8.0, 2.5 mM MgCl₂, 0.1 mM EDTA, 10 mM NaHCO₃, 10 mM NaHSO₃, and 10 mM 2-mercaptoethanol. RuBP-dependent ¹⁴CO₂ fixation in these extracts was measured as described below. The RUBISCO was first activated by addition of MgCl₂ and NaHCO₃ and incubated at room temperature for one h. Reactions (30 μL) were performed in 1.5 mL capacity polypropylene tubes. The mixture consisted of 15 μL extract (diluted as needed with 0.1 M NaEPPS, pH 8, containing 20 mM MgCl₂, 20 mM NaHCO₃, 1.0 mM EDTA, 50 μg/mL bovine serum albumin, and 2 mM dithiothreitol). Ten microliters of a solution of [¹⁴C]—NaHCO₃ (ca. 0.3 mM) was added and the reaction was started by addition of 5 μL of 6.0 mM RuBP at 25° C. Three assays containing different levels of highly active extracts were performed for 10 min, after which 25 μL of the reaction was transferred to a 7 mL glass vial containing 0.4 mL 10% v/v acetic acid. Two pairs of reactions were performed for less active samples. Each reaction containing RuBP was paired with another lacking RuBP, and reactions were terminated at 10 min and 60 min. Three controls, each with excess enzyme for determination of the specific radioactivity of ¹⁴C in the assay and no enzyme, were performed with each set of assays. The vials containing quenched reactions were taken to dryness on a hotplate, and taken up in 0.2 mL water. Ecolume scintillation fluid (5.0 mL) (MP Biologicals, Solon, Ohio) was added to the samples and the tubes were capped and the radioactivity was determined in a Beckman LS6000TA liquid scintillation counter. The specific activity of the ¹⁴C in the assay was calculated by subtracting the mean of the no-enzyme controls from the excess enzyme controls, averaging the result, and dividing by 25 nmol RuBP added to the aliquot collected. For active samples, the no-enzyme value was subtracted from the observed counts, and the corrected value converted to nmol ¹⁴C fixed. For less-active samples, the counts in the −RuBP reaction of each pair was subtracted from the corresponding +RuBP reaction. If the difference was considered meaningful (at least 50% higher +RuBP, in both samples), nmol ¹⁴C fixed was calculated as above. The results were then converted to nmoles/min/mg of protein (mU/mg) taking into account the volume of extract in each assay.

It is understood, as those skilled in the art will appreciate, that the invention encompasses more than the specific exemplary sequences. Alterations in a nucleic acid fragment which result in the production of a chemically equivalent amino acid at a given site, but do not affect the functional properties of the encoded polypeptide, are well known in the art. For example, a codon for the amino acid alanine, a hydrophobic amino acid, may be substituted by a codon encoding another less hydrophobic residue, such as glycine, or a more hydrophobic residue, such as valine, leucine, or isoleucine. Similarly, changes which result in substitution of one negatively charged residue for another, such as aspartic acid for glutamic acid, or one positively charged residue for another, such as lysine for arginine, can also be expected to produce a functionally equivalent product. Nucleotide changes which result in alteration of the N-terminal and C-terminal portions of the polypeptide molecule would also not be expected to alter the activity of the polypeptide. Each of the proposed modifications is well within the routine skill in the art, as is determination of retention of biological activity of the encoded products.

### PREFERRED EMBODIMENTS OF THE INVENTION

In one embodiment, the chloroplast-encoded A. edulis RUBISCO LSU full length coding sequence (AErbcL) was amplified directly from A. edulis total DNA in a PCR reaction. Using a pBS-based plasmid the A. edulis LSU was linked with a tomato RUBISCO SSU transit peptide coding sequence at its 5′end and a 6-His tag coding sequence at its 3′end, forming the nAErbcL nuclear transgene. The nucleus-encoded A. edulis RUBISCO SSU mature protein coding sequence (AEebcS) was amplified from A. edulis mRNA by a RT-PCR approach. AErbcS was also linked with a tomato RUBISCO SSU transit peptide coding sequence at its 5′end but a HA tag coding sequence at its 3′end, forming the nAErbcS nuclear transgene.

In another embodiment the nAErbcL and nAErbcS sequences were isolated from their host pBS plasmids. The nAErbcL fragment was cloned into the pREC10 plasmid and the resultant pREC102 harbored a chimeric transgene expression cassette of SCP1 Pro::nAErbcL::Pha Ter. The nAErbcS fragment was cloned into the pREC11 plasmid. The resultant pREC1104 contained a chimeric transgene expression cassette of SCP1 Pro::nAErbcS::NOS Ter. These two chimeric transgenes were then constructed into a pZBL1 M1-based expression plasmid, individually or in combination, resulting in three expression plasmids for tobacco nuclear transformation: pRBI104 (containing nAErbcL), pRBI105 (containing nAErbcS) and pRBI106 (containing both nAErbcL and nAErbcS).

In another embodiment, the transgene cassettes in pRBI104, pRBI105, and pRBI106 were introduced into Nicotiana tabacum (tobacco) nuclear genomes by a standard Agrobacterium-mediated transformation approach. Expression of nAErbcL and nAErbcS was analyzed with immunoblot assays, using total soluble protein (TSP) extracts of each transformant. The products of nAErbcL and nAErbcS were detected by an Anti-His (C-term)-HRP antibody and an Anti-HA-HRP antibody, respectively. The results confirmed transgene expression, precursor translocation, and mature protein accumulation in the transformants. It also demonstrated that, in the tobacco chloroplast, the A. edulis RUBISCO subunits could assemble into a normal RUBISCO complex with either other A. edulis RUBISCO subunits or with tobacco RUBISCO subunits.

In another embodiment, two chimeric transgene expression cassettes; SCP1 Pro::nAErbcL::Pha Ter in pREC102 and SCP1 Pro::nAErbcS::NOS Ter in pREC1104 were isolated and inserted into a pZSL222-based soybean expression vector individually or in combination. This resulted in three soy nuclear expression plasmids: pRST106 (containing the nAErbcL construct), pRST107 (containing both the nAErbcL and nAErbcS constructs), and pRST108 (containing only the nAErbcS construct). The pRST107 and pRST108 plasmids were introduced into the soybean nuclear genome by biolistic bombardment transformation. Transgene expression, precursor translocation, mature protein accumulation, and RUBISCO complex assembly were demonstrated by immunoblot analysis, using total soluble protein (TSP) extracts of each transformant.

In another embodiment, the cpAErbcL chloroplast transgene, encoding the A. edulis LSU with a C-terminal 6-His tag, was amplified from pRBI104 by PCR. The cpAErbcL fragment was cloned into the pTCP10 plasmid. The resultant pTCP11 harbored a chimeric transgene expression cassette of psbA Pro::cpAErbcL::rps16 Ter. The construct was further isolated and inserted into a master chloroplast transformation vector pTCP101, producing pTCP103 for AErbcL-6His expression in tobacco chloroplasts

In another embodiment, the transgene in pTCP103 was introduced into the tobacco chloroplast genome of an rbcL-KO tobacco, by biolistic bombardment transformation. The rbcL-KO tobacco lacked an intact rbcL gene, and thus had no background RUBISCO activity. Transgene expression, determined using immunoblot assay, indicated a high level of expression (average of 1.66% TSP with the highest accumulation of 4% TSP). Further analysis demonstrated that the heterologous A. edulis LSU had formed a RUBISCO complex with the endogenous tobacco SSU, generating the enzymatic activity of a functional RUBISCO.

In another embodiment, the Griffithsia monilis RUBISCO rbcL gene was provided in pGm-rbcLS-TVE, a plasmid containing a genomic fragment of G. monilis. The GMNrbcL-6His coding sequence was synthesized by PCR using the plasmid as template and further integrated into a pBS-based plasmid to translationally fuse it to a tomato RUBISCO SSU transit peptide coding sequence. This resulted in pBS-nGMNrbcL containing nuclear transgene nGMNrbcL The nGMNrbcL fragment was isolated from pBS-nGMNrbcL and cloned into the pREC10 plasmid such the resultant pREC103 harbored a chimeric transgene expression cassette of SCP1 Pro::nGMNrbcL::Pha Ter. This chimeric transgene was then inserted into a pZBL1M1-based expression plasmid resulting in pRBI107. The cpGMNrbcL transgene, without the tomato SSU transit peptide, was amplified from pRBI107 by PCR and inserted into the pTCP10 plasmid. The resultant pTCP12A harbored a chimeric transgene expression cassette of psbA Pro::cpAErbcL::rps16 Ter. The construct was further isolated and inserted into a master chloroplast transformation vector pTCP101, producing pTCP104 for GMNrbcL-6His expression in tobacco chloroplasts

In another embodiment, the transgene in pTCP104 was introduced into the tobacco chloroplast genome of an rbcL-KO tobacco by biolistic bombardment transformation. Transgene expression, determined using an immunoblot assay, showed a low level of expression (average of 0.04% TSP with the highest accumulation of 0.19% TSP). The expression product was purified using a Ni-NTA column. Further analysis demonstrated that the heterologous G. monilis LSU had formed a RUBISCO complex with the endogenous tobacco SSU, generating the enzymatic activity of a functional RUBISCO.

### EXAMPLES

The present invention is further illustrated in the following Examples. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications to the invention to adapt it to various usages and conditions. Thus, various modifications of the invention in addition to those shown and described herein will be apparent to those skilled in the art from the foregoing description. Such modifications are also intended to fall within the scope of the appended claims.

Additional abbreviations used in the following Examples are as follows: “mm” means millimeter, “h” means hour(s), “min” means minute(s), “day” means day(s), “mU/mg” means milli unit per milligram, “mL” means milliliters, “mg/mL” means milligram per milliliter, “L” means liters, “μL” means microliters, “mM” means millimolar, “nmoles” means nano moles, “Cm” means centimeters, “mg/L” means milligram per liter, “μg/mL” means microgram per milliliter, “g” means gram, “g/L” means gram per liter, “mL/L” means milliliter per liter, “μM” means micromolar, “ng” means nano grams, “μg” means micrograms, “° C.” means degrees Centigrade, “bp” means base pair, “bps” means base pairs, “nt” means nucleotide, “kd” means kilodaltons., “psi” means per square inch, “kpb” means kilobase pair, “v/v” means volume per volume, “sec” means second, “dpm/nmol” means disintegration per minute per nanomole, “SDEV” means standard deviation, “kV” means kilovolt, “mA/cm²” means milliamp per square centimeter.

### Example 1

### Cloning of Amaranthus edulis RUBISCO LSU and SSU Coding Sequences

Seeds of Amaranthus edulis (also known as A. caudatus) (obtained from Geo. W. Park seed Co, Inc, Greenwood, S.C.) were germinated and plants were grown in the greenhouse with natural light at 28° C. for five weeks. Total DNA and total RNA were isolated from the leaf tissue using the DNeasy and RNeasy Plant Mini Kits (Qiagen, Valencia, Calif.). PolyA-mRNA was purified from total RNA using the Oligotex mRNA Kit (Qiagen, Valencia, Calif.) as recommended by the manufacturer. DNA and RNA concentrations were determined by using a Nanodrop ND-1000 as described by Nanodrop Technologies (Montchanin, Del.).

Although the chloroplast genome of A. edulis has not been previously sequenced and the LSU and SSU subunits of its RUBISCO coding sequences have not been cloned, comparison of the rbcL genes of A. hypochondriacus, A. tricolor, corn, soy, and tobacco indicated that all these sequences were highly conserved at both the amino acid and nucleotide levels. The sequences of the A. hypochondriacus and A. tricolor rbcL genes were almost identical (99.3%). PCR primers, rbc34 (SEQ ID NO:1) and rbc35 (SEQ ID NO:2), synthesized based on the rbcL sequence of the A. hypochondriacus gene, were used to amplify the chloroplast-encoded RUBISCO LSU full length coding sequence (start codon to stop codon) from the A. edulis total DNA. A 25-μL PCR reaction consisting of 100 ng total DNA, 10 pmoles each of rbc34 and rbc35, 5 nmoles of each dNTP, 2.5 units of Pfu ultra enzyme and 2.5 μL Pfu ultra buffer (Stratagene, La Jolla, Calif.) was used. The reaction was pre-heated at 95° C. for 4 min, followed by 30 cycles of denaturing at 95° C. for one min, annealing at 56° C. for one min, extending at 72° C. for 2 min and ended by incubating at 72° C. for 10 min. The product was purified using a QIAquick Gel Extraction Kit (Qiagen) and cloned into a PCR Blunt II TOPO vector using a Zero Blunt TOPO PCR Cloning Kit (Invitrogen, Carlsbad, Calif.) as recommended by the manufacturer. The inserted fragment was sequenced and the resultant plasmid, (pTP-AErbcL), contained a 1,428 bp insert which consisted of a full length A. edulis rbcL gene that encoded a 475-amino acid RUBISCO LSU. This gene was nearly identical to the A. hypochondriacus (99.6%) and A. tricolor (99.7%) rbcL's, and its translated product was 99.2% and 99.4% identical to A. hypochondriacus and A. tricolor RUBISCO LSU's, respectively. SEQ ID NO:25 depicts the sequence of the A. edulis rbcL gene.

A degenerate primer rbc36 (SEQ ID NO:3) was designed based on the protein sequence from −1 Cys to +6Pro of the three mature SSU coding sequences of RUBISCO from A. hypochondriacus. Position +1 represents the Met at the beginning of the RUBISCO SSU mature protein of A. hypochondriacus. The primer covered most of the diversity in this region of the three genes and was used to amplify the mature SSU coding sequence. To prepare the cDNA, a 20-μL reverse transcription reaction was prepared using the Omniscript RT kit (Qiagen), which consisted of 20 ng polyA-mRNA of A. edulis, 1.0 μL of 5 mM dNTP, 2.0 μL of 1.0 μM degenerate oligo-dT (a mixture of A-dT₁₅, C-dT₁₅, and G-dT₁₅), 10 units RNase inhibitor, 2.0 μL 10× reverse transcriptase buffer, and 4.0 unit of reverse transcriptase. The reaction was carried out at 37° C. for one h. Then, 3.0 μL of this reaction mixture was used to prepare a 25-μL PCR reaction by mixing with 10 pmole rbc36 and oligo dT, 5 nmole each of dNTPs, 2.5 units of Pfu ultra enzyme and 2.5 μL Pfu ultra buffer (Stratagene, La Jolla, Calif.). The PCR reaction was preheated at 95° C. for 4 min, followed by 30 cycles of denaturing at 95° C. for 0.5 min, annealing at 56° C. for 0.5 min, extending at 72° C. for one min and ended by incubating at 72° C. for 10 min. The product was purified using a QIAquick Gel Extraction Kit (Qiagen) and cloned into a PCR Blunt II TOPO vector using the Zero Blunt TOPO PCR Cloning Kit (Invitrogen) as recommended by the manufacturer. Sequencing of inserts from more than 10 of the resulting plasmids showed that they were identical to each other. This sequence contained an open reading frame, a 3′ UTR sequence and a polyA tail. The ORF sequence encoded a protein which was 100%, 96.8%, and 96% identical to the mature RUBISCO SSU-1, SSU-2, and SSU-3 of A. hypochondriacus respectively and it was concluded that the sequence represented an A. edulis rbcS gene. Using the A. edulis rbcS coding sequence, two non-degenerate primers were synthesized to redo the PCR reaction for synthesizing the rbcS coding sequence from the cDNA. Primers rbc52 (SEQ ID NO:4) and rbc53 (SEQ ID NO:5) amplified a 375-nt coding sequence of AErbcS, which encoded a 124-amino acid mature RUBISCO SSU of A. edulis, starting from +1 Met to +124Leu and ending with a translation stop codon. AErbcS was purified using the QIAquick Gel Extraction Kit (Qiagen) and cloned into a PCR Blunt II TOPO vector using the Zero Blunt TOPO PCR Cloning Kit (Invitrogen), generating pTP-AErbcS. The presence of AErbcS in the vector was confirmed by sequencing. SEQ ID NO:26 depicts sequence of the mature SSU coding region (AErbcS) of the A. edulis rbcS gene. At the DNA level, this coding sequence was 99.2%, 94.1 %, and 93.3% identical to the coding regions of the A. hypochondriacus rbcS1, rbcS2, and rbcS3 genes, respectively. The translated product was 100%, 96.8%, and 96% identical to the mature RUBISCO SSU-1, SSU-2, and SSU-3 of A. hypochondriacus, respectively.

### Example 2

### Construction of Amaranthus edulis RUBISCO Transgenes for Tobacco Nuclear Expression

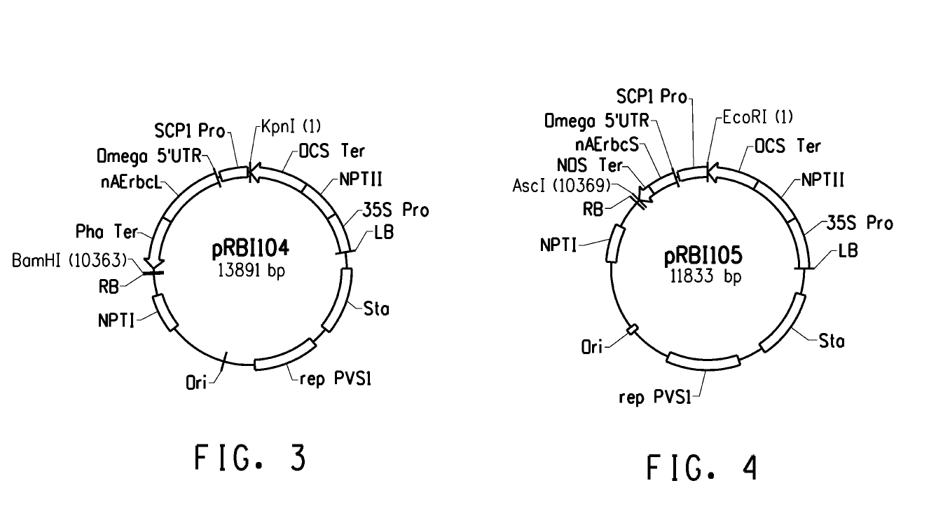

Although the AErbcL gene originated from the chloroplast genome, most of its codons matched the common nuclear codon usage of higher plants. Since AErbcS in the pTP-AErbcS vector, was a nuclear gene, both AErbcL and AErbcS could therefore be directly used for nuclear transformation. AErbcL contained a KpnI site GGTACC for Gly331 and Thr332 while the AErbcS coding sequence contained an EcoRI site GAATTC for Glu43 and Phe44. Because both sites could later interfere with the cloning procedure, they were mutated using a Quikchange site-directed mutagenesis kit (Stratagene, La Jolla, Calif.) as recommended by the manufacturer. The KpnI site in AErbcL was changed to GGAACC using primers rbc61 (SEQ ID NO:6) and rbc62 (SEQ ID NO:7). The EcoRI site in AErbcS was changed to GAGTTC using primers rbc56 (SEQ ID NO: 8) and rbc57 (SEQ ID NO: 9). These sequence changes did not affect the encoded peptide sequences. Subsequently, the modified pTP-AErbcL was used as a template to synthesize an AErbcL-6His coding sequence in a standard PCR reaction as described above. The reaction used primers rbc59 (SEQ ID NO:l0) and rbc60 (SEQ ID NO:11). In this sequence, a 6His-tag coding region was added into AErbcL just before the stop codon and a NotI site was also created after the stop codon. The first 3 amino acids (Met-Ser-Pro) of AErbcL were removed and an MscI site was created. AErbcL-6HIS was cloned into pCR-Blunt II-TOPO, making pTP-AErbcL-6HIS. The modified pTP-AErbcS was also used as a template to synthesize in a standard PCR reaction an AErbcS-HA coding sequence containing an N-terminal HA tag. The reaction used primers rbc52 (SEQ ID NO:4) and rbc58 (SEQ ID NO:12). In this PCR reaction, a HA-tag (YPYDVPDYA) coding region was added to AErbcS just before the stop codon. A NotI site was also created after the stop codon. This sequence had an MscI site at the fourth amino acid residue of AErbcS. AErbcS-HA was cloned into pCR-Blunt II-TOPO, creating pTP-AErbcS-HA. Finally, the AErbcL-6HIS and AErbcS-HA coding sequences were isolated from these plasmids through MscI/NotI digestion, and translationally fused to a tomato RUBISCO SSU transit peptide coding sequence in a pBS plasmid, creating pBS-nAErbcL (SEQ ID NO: 27) and pBS-nAErbcS (SEQ ID NO:28), respectively. In pBS-nAErbcL, the nAErbcL fragment (FIG. 3) was a 1,623-bp coding sequence encoding a 540-amino acid fusion protein, consisting of a tomato transit peptide, an A. edulis RUBISCO LSU, and a C-terminal 6His-tag. In pBS-nAErbcS, the nAErbcS fragment (FIG. 4) was a 573-bp coding sequence encoding a 190-amino acid fusion protein, consisting of a tomato transit peptide, an A. edulis mature RUBISCO SSU, and a C-terminal HA-tag.

To express nAErbcL and nAErbcS in a nuclear transformation approach, both sequences were isolated from their host pBS plasmids by StuI/NotI digestion and purified using QIAquick Gel Extraction Kits. The nAErbcL fragment was then cloned into pREC1 0 plasmid between the SmaI and NotI sites. Since pREC10 is a pBS (pBluescript SK+) based plasmid containing a synthetic SCP1 promoter with an omega 5′UTR (SCP1 Pro) and a soybean phaseolin terminator (Pha Ter), the resultant pREC102 harbored a chimeric transgene expression cassette of SCP1 Pro::nAErbcL::Pha Ter (FIG. 1).

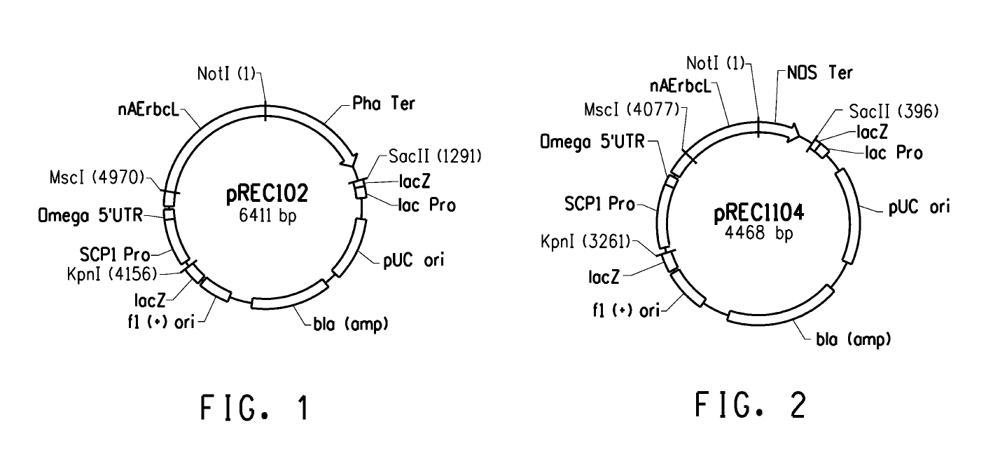

The nAErbcS was cloned into the pREC11 plasmid between the SmaI and NotI sites. The structure of pREC11 was similar to that for pREC10, except that the Agrobacterium T-DNA NOS terminator (NOS Ter) was substituted for Pha Ter. The resultant plasmid, containing a chimeric transgene expression cassette of SCP1 Pro::nAErbcS::NOS Ter, was named pREC1104 (FIG. 2).

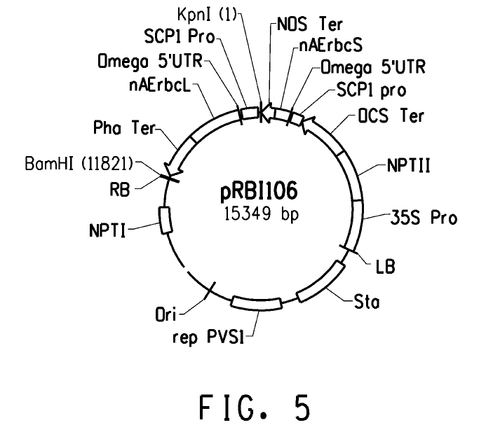

These two chimeric transgenes were then inserted into a pZBL1M1-based expression plasmid for tobacco nuclear genome transformation. pZBL1M1 is a master tobacco binary vector containing a marker gene, CaMV 35S promoter (35S Pro)::NPTII::Agrobacterium T-DNA OCS terminator (OCS Ter). A standard digestion, gel-purification, and sub-cloning procedure was followed for making the constructs. SCP1 Pro::nAErbcL::Pha Ter was isolated from pREC102 by KpnI/BamHI digestion and inserted into pZBLM1 to form pRBI104 (FIG. 3). SCP1 Pro::nAErbcS::NOS Ter was isolated from pREC1104 by EcoRI/AscI digestion and inserted into pZBLM1 to form pRBI105 (FIG. 4). SCP1 Pro::nAErbcL::Pha Ter isolated earlier from pREC102 by KpnI/BamHI digestion was inserted into pRBI105 between BamHI and KpnI, to form pRBI106 (FIG. 5).

### Example 3

### Expression of Amaranthus edulis RUBISCO Transgenes in Tobacco Nuclear Genomes

### Tobacco Nuclear Transformation

Plasmids pRBI104, pRBI105, and pRBI106 were introduced into wild type Nicotiana tabacum (tobacco) nuclear genomes by a standard Agrobacterium-mediated transformation approach. In the first step of the procedure, 1.0 μg plasmid DNA was electroporated into competent Agrobacterium strain LBA4404 (Invitrogen) in a 2 mm cuvette at 2.5 kV using a TransPorator Plus device (BTX, San Diego, Calif.). The Agrobacterium was grown up overnight in MinA medium (1 % Bacto tryptone, 1% yeast extract, 0.5% NaCl) containing 50 mg/L kanamycin, washed with Gibco BRL MS medium (Invitrogen, Carlsbad, Calif.) with 3% sucrose, and resuspended in twice the volume of MS. A sterile wild type tobacco leaf disc (one cm in diameter) was then infected by placing it in the Agrobacterium suspension for 30 min, after which it was transferred onto a shoot induction plate (MS medium with 0.7% agar, 0.1 mg/L 1-naphthaleneacetic acid (NAA), 1 mg/L Benzylaminopurine (BAP), and 1.0 mL/L 1,000×vitamins) for 3 days. After 3 days, the disc was washed in 30 mL MS medium containing 500 mg/L cefotaxime (Calbiochem, San Diego, Calif.) for 20 min, and then placed on a shoot induction plate containing 300 mg/L kanamycin and 500 mg/L cefotaxime for 3 weeks. The leaf disc was then transferred to a new shoot induction plate to allow callus growth and shoot regeneration. Finally, for root regeneration, rootless shoots (one cm high) were transplanted to M404 medium (Phytotechnology Labs, Shawnee Mission, Kans.), supplemented with 0.7% agar, 300 mg/L kanamycin, 500 mg/L cefotaxime, 100 mg/L myo-Inositol, 1 mg/L nicotinic acid, 1 mg/L pyrixidine HCl, and 10 mg/L thiamine HCl. Transgenic plants, transformed by pRBI104 (16 plants), pRBI105 (24 plants), and pRBI106 (24 plants), were transplanted into Metro Mix soil (Griffin Greenhouse Supply, Morgantown, Pa.) and grown under regular conditions in plant growth chambers (14 h light/10 h dark, at 28° C.).

### Expression of RUBISCO Transgenes

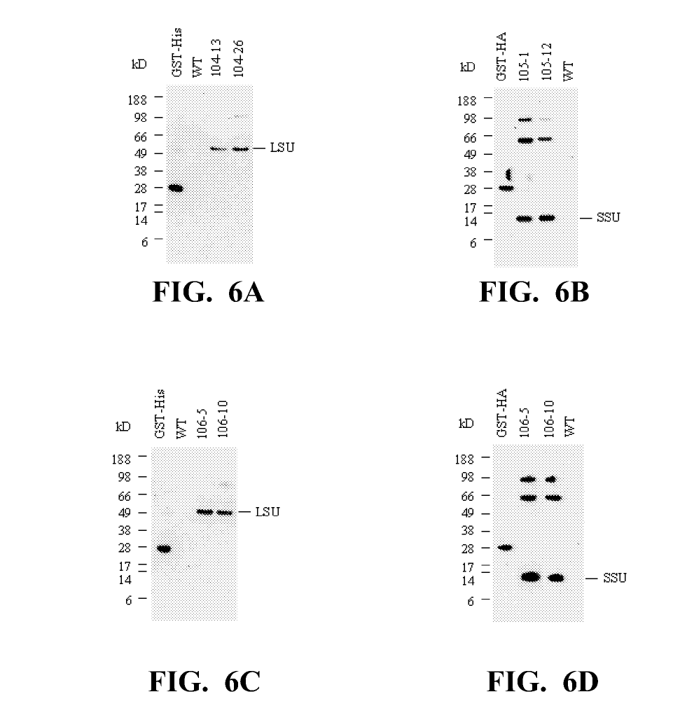

Based on their transgene structures, the PRBI104 plants should produce a 54 kD 6-His tagged A. edulis RUBISCO LSU (i.e. LSU-6His), a chloroplast-accumulated product of nAErbcL. The pRBI105 plants should produce a 15.6 kD HA tagged A. edulis RUBISCO SSU (i.e. SSU-HA), a chloroplast-accumulated product of nAErbcS. The pRBI106 plants should produce both 6-His tagged A. edulis RUBISCO LSU and HA tagged A. edulis RUBISCO SSU. To examine expression of these constructs, total soluble protein (TSP) extracts of each transformant were prepared by grinding 150 mg leaves of one-month soil grown transformants in 200 μL of ice-cold leaf extraction buffer. The buffer contained 50 mM Tris-HCl (pH 8.0), 50 mM NaCl, 0.1 mM EDTA, 2 mM DTT, 5 mM MgCl₂, 5% glycerol, and 1% plant protease inhibitor cocktail (Sigma, St. Louis, Mo.). Cell debris was removed by centrifugation at 10,000×g at 4° C. for 15 min and protein concentration in the supernatant was determined using the Coomassie Plus Protein Assay Reagent (Pierce Co., Rockford, Ill.). Protein extracts containing 6 μg TSP were analyzed by SDS-PAGE on a 4%-12% NuPAGE Novex Bis-Tris Gel (Invitrogen, Carlsbad, Calif.). Sample pre-treatment and electrophoresis were conducted using NuPAGE reagents following the NuPAGE Technical Guide (Invitrogen). The separated proteins were transferred from the NuPAGE SDS gel to a nitrocellulose membrane (Invitrogen) using a Pharmacia-LKB 2117 multiphor II (Pharmacia Biotech, Piscataway, N.J.), sandwiched by 2 layers of Whatman #1 filter paper on the both sides. The gel and filter were moistened with semi-dry western transfer buffer (40 mM glycine, 50 mM Tris, 1.0 mM SDS, and 20% methanol) and the transfer was carried out at 0.8 mA/cm² for 1.5 h. Protein blots were probed with 1,000× diluted Anti-His (C-term)-HRP Antibody (Invitrogen) for LSU-6His (in the case of pRBI104 and pRBI106 transformants) and with 5,000× diluted Anti-HA-HRP (Sigma) for SSU-HA (in pRBI105 and pRBI106 transformants). Signals were detected with SuperSignal West Pico Chemiluminescent Substrate Solution (Pierce Colo.) in a standard Western blot assay and recorded using a Lumi-Imager (Roche Diagnostics, Indianapolis, Ind.). These analyses showed that 11 PRBI104 transformants and 20 PRBI106 transformants accumulated LSU-6His. In addition, 10 pRBI105 transformants and 13 pRBI106 transformants accumulated SSU-HA. Amongst the PRBI106 transformants, 11 plants accumulated both LSU-6His and SSU-HA. The accumulated LSU-6His and SSU-HA had molecular masses of 54 kD and 15.6 kD, respectively indicating that both the LSU-6His and the SSU-HA had been translocated into chloroplast and their transit peptides had been removed from the precursor (FIG. 6). In the SSU-HA transformants pRST105 and pRST106, two larger proteins of unknown identity were also detected using the Anti-HA HRP antibody (FIGS. 6B and 6D).

The 6-His tag and HA tag (YPYDVPDYA) coding sequences were fused separately to the end of a GST coding sequence in pGSTf (Qi, M., et al., Biopolymers: Peptide Science, 90, 28-36, 2008). The resultant plasmids were transformed into BL21 E. coli to produce GST-6His and GST-HA proteins. GST-6His was purified on a Ni-NTA column (Qiagen, Valencia, Calif.) and used (140 ng) as a control in immunoblot assays of LSU-6His. GST-HA was purified on a glutathione-agarose column and used (14 ng) as a control in immunoblot assays of SSU-HA. Concentrations of LSU-6His and SSU-HA were calculated by measuring signal intensities of the control and the sample proteins. In pRBI104 transformants, LSU-6His accumulated from 0.004% to 0.01% TSP, while in pRBI105 transformants the SSU-HA level was from 0.03% to 1% TSP. In pRBI106 transformants, LSU-6His was from 0.1% to 0.4% TSP, and accumulation of SSU-HA was from 0.7% to 3.5% TSP.

### Assembly of RUBISCO Complex

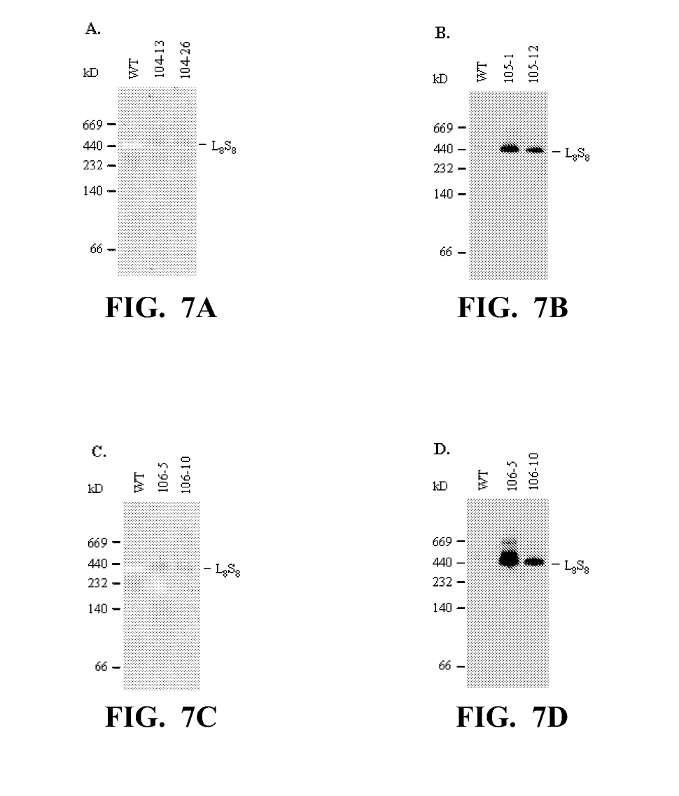

Assembly of the L₈S₈ complex in chloroplasts stabilizes both LSU and SSU and leads to its accumulation at high levels (Rodermel, S., Photosynthesis Res., 59:105-123, 1999). In the pRBI104 and pRBI105 transgenic tobacco lines described above, products of nAErbcL and nAErbcS were targeted to the chloroplasts, suggesting they might be interacting with the endogenous tobacco RUBISCO subunits leading to formation of hybrid RUBISCO complexes. To confirm this hypothesis, leaf protein extracts containing 6 μg protein were analyzed by Native-PAGE on a 10% Novex Tris-Glycine Gel (Invitrogen, Carlsbad, Calif.). Sample pre-treatment and electrophoresis were performed using Invitrogen native electrophoresis reagents following the Novex Pre-Cast Gel Technical Guide (Invitrogen). The separated proteins and protein complexes were transferred from the gel to a nitrocellulose membrane and analyzed by Western blot. Anti-His (C-term)-HRP Antibody (Invitrogen) and Anti-HA-HRP Antibody (Sigma) were used to detect LSU-6His in pRBI104 and pRBI106 and SSU-HA in pRBI105 and pRBI106, respectively, as described above and results were recorded using a Lumi-Imager. Both antibodies demonstrated the presence of a complex of about 550 kD in the transformant samples, the same size as the native RUBISCO L₈S8 complex (FIG. 7). Since the Anti-His (C-term)-HRP Antibody had a relatively lower titer and the endogenous tobacco RUBISCO strongly interfered in the reaction (note the white areas in FIGS. 7A and 7C), the signals for the L₈S₈ complexes were visible but weak.

Since in the pRBI104 transformants there is only an A. edulis LSU and no SSU and in the PRBI105 transformants there is only an A. edulis SSU and no LSU, the presence of the normal-sized complex in these lines indicates that the A. edulis subunits have assembled into a hybrid complex with the tobacco subunits. In the pRBI106 transformants, tobacco and A. edulis LSU and SSU may form a mix of hybrid and non-hybrid RUBISCO complexes or they may preferentially assemble only with the subunits from the same species, thus forming a mixture of non-hybrid A. edulis and tobacco complexes.

### Example 4

### Construction of Amaranthus edulis RUBISCO Transgenes for Soybean Nuclear Expression

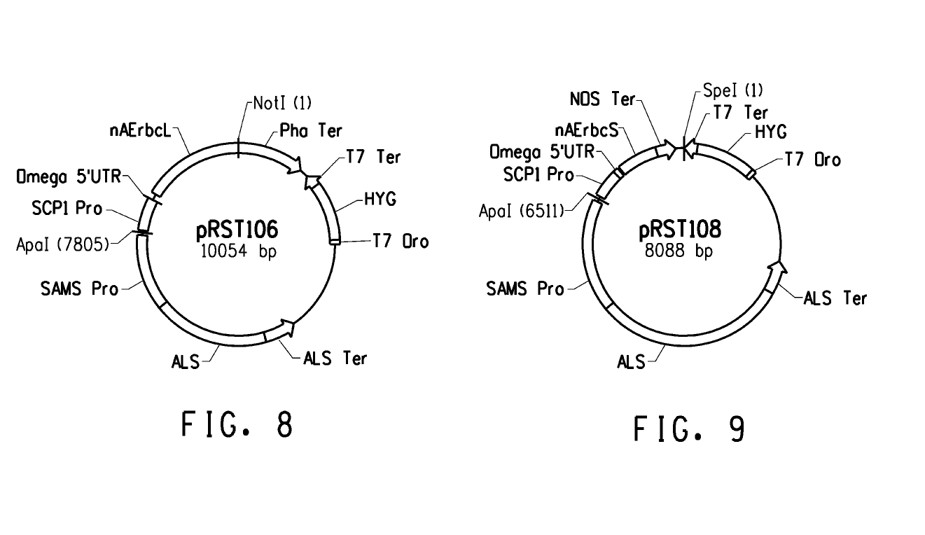

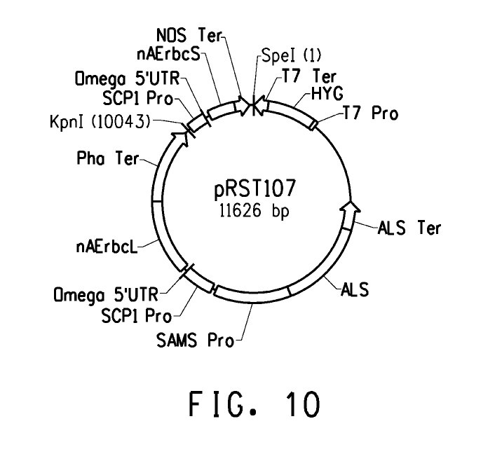

To express A. edulis RUBISCO genes in the soybean nucleus, two chimeric transgene expression cassettes; SCP1 Pro::nAErbcL::Pha Ter in pREC102 and SCP1 Pro::nAErbcS::NOS Ter in pREC1104 were inserted into a pZSL222-based expression plasmid. The pZSL222 is a master soybean expression vector containing a marker gene of soybean SAMS promoter (SAMS Pro)::ALS::soybean ALS terminator (ALS Ter) (see U.S. Pat. No. 7,217,858 for selective marker structure SAMS PRO::ALS::SAMS Ter). Standard digestion, gel-purification, and sub-cloning procedures were used for generating the constructs. Initially, SCP1 Pro::nAErbcL::Pha Ter was isolated from pREC102 by ApaI/NotI digestion and inserted into pZSL222 to form pRST106 (FIG. 8). Then, SCP1 Pro::nAErbcS::NOS Ter was isolated from pREC1104 by ApaI/SpeI digestion and inserted into pZSL222 to form pRST108 (FIG. 9). Finally, SCP1 Pro::nAErbcS::NOS Ter isolated from pREC1104 by KpnI/SpeI digestion was inserted into pRST106 to form pRST107 (FIG. 10).

### Example 5

### Expression of Amaranthus edulis RUBISCO Transgenes in Soybean Nuclear Genome

### Soybean Nuclear Transformation

Plasmids pRST107 and pRST108 were introduced into the soybean nuclear genome by biolistic bombardment transformation (Finer, J. J., and McMullen, M. D. In vitro Cell Dev. Biol., 27 p., 175-182, 1991; and Stewart, C. N., et al., Plant Physiol., 112, 121-129, 1996). Briefly, embryogenic suspension cultures of Glycine max Merrill (cultivar “Jack”) were initiated and maintained in MS medium modified according to Samoylov and co-workers (Samoylov, V. M., et al., In Vitro Cell Dev. Biol.—Plant, 34, 8-13, 1998). This was accomplished by placing about 5 to 10 small embryogenic clusters (with a diameter of about 0.5 to 1.0 mm) in a 250 ml flask containing 50 mL of the liquid modified MS medium. The flasks were maintained on a gyratory shaker at 26° C. under cool white fluorescent lights with a 16/8 h day/night photoperiod. The embryogenic cultures were then subcultured on a bi-weekly basis by selecting 5 to 10 embryogenic clusters and transferring these to fresh medium.

The particle bombardment method (Klein et al., Nature, 327, 70-73, 1987, U.S. Pat. No. 4,945,050) was used to genetically transform the soybean embryogenic cultures. Freshly subcultured samples were bombarded using the Bio-Rad PDS-1000/He Particle Bombardment System (Bio-Rad, Hercules, Calif.) with plasmid DNA-coated gold particles (average diameter 0.6 μm). The gold particles were coated with DNA using the following procedure. To 50 μL of a 60 mg/mL 1.0 μm gold particle suspension was added (in order): DNA (5 μL from a 1.0 μg/μL solution), 20 μL spermidine (0.1 M), and 50 μL CaCl₂ (2.5 M). The particle preparation was then agitated for three minutes, spun in a microfuge for 10 seconds and the supernatant removed. The DNA-coated particles were then washed once in 400 μL 70% ethanol and resuspended in 40 μL of anhydrous ethanol. The DNA/particle suspension was sonicated three times for one second each. The DNA-coated gold particles (5 μL) were then loaded on each macro carrier disk.

To accomplish gene transfer by particle bombardment, about 100 mg of embryogenic tissue was transferred to the center of an empty Petri dish. The tissue was allowed to air dry by placing the plate without cover in a laminar flow hood. Following the drying treatment, the tissue was placed into the chamber of the Bio-Rad PDS-1000/He Particle Bombardment System at a distance of about 3.5 inches from the stopping screen. Air in the chamber was then removed to provide a partial vacuum of about 28 inches of Hg. A helium burst pressure of 1100 psi was used to accelerate the DNA-coated particles into the tissue. For each bombardment, 0.3 mg gold particles coated with 0.03 μg DNA was used. After bombardment, the tissue was grown for 7 days in the liquid MS medium. The liquid medium was then replaced by a similar medium containing 100 ng/mL of chlorsulfuron as the selection agent. This selective medium was refreshed weekly for about 7 to 8 weeks. Seven to eight weeks post bombardment, green, transformed tissue was observed growing from untransformed, necrotic embryogenic clusters. The isolated transformed tissue was cultured in 50 mL of modified liquid MS medium containing 100 ng/mL chlorsulfuron in a 250 mL flask with rotary shaking. To regenerate transgenic soybean plantlets, transformed embryogenic clusters were removed from liquid modified MS medium and blotted on sterile filter paper. Tissue clumps were broken or gently squashed with forceps and placed on solid regeneration medium SB166 for one week. Following this one week incubation, 10 to 20 embryogenic clusters were transferred to a Petri dish containing SB103 medium for 3 weeks.

After the four weeks maturation period, individual embryos are desiccated by placing about 50 to 100 embryos into an empty petri dish (60×25 mm) for approximately 7 to 10 days. Desiccated embryos are transferred to germination medium by first breaking up the mature embryo clusters and transferring individual embryos to the germination medium. Shoots and roots then form from each embryo and these plantlets can be transferred to soil.

Eighteen plantlets obtained from the bombardment with pRST107 and 26 from pRST108 were transplanted into Metro Mix soil and grown in a growth chamber under 16/8 h photoperiod at 26/24 C day/night temperatures.

### Expression of RUBISCO Transgenes

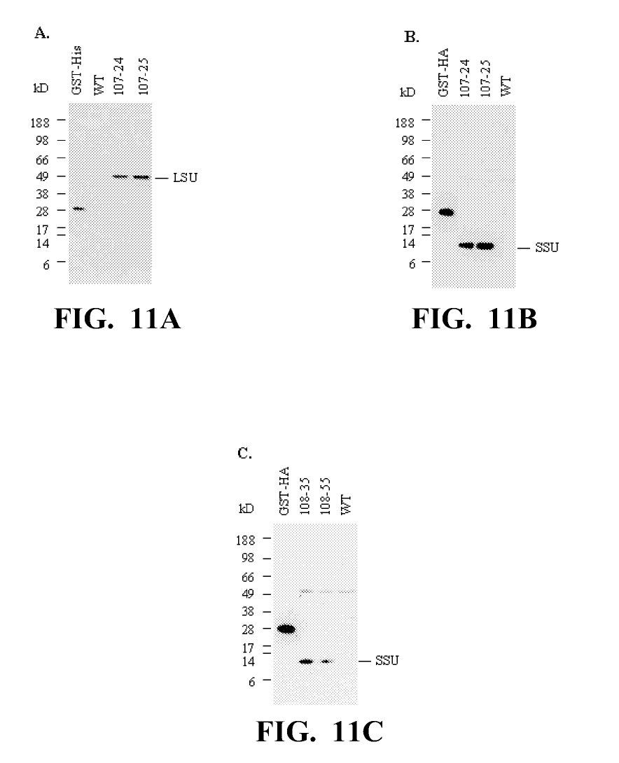

Since pRST107 plants were transformed by both nAErbcS and nAErbcL, they were expected to produce both 54 kD A. edulis RUBISCO LSU-6His and 15.6 kD A. edulis RUBISCO SSU-HA proteins. However, pRST108 transformants were expected to produce only the SSU-HA protein since it possessed only the nAErbcS transgene. To confirm this hypothesis, protein extracts were prepared from one-month soil grown transformants by grinding 150 mg leaves in 200 μL of ice-cold leaf extraction buffer (components shown earlier). Protein samples (6 μg of each) were analyzed by SDS-PAGE and Western blot assay, as described above. The Anti-His (C-term)-HRP antibody was used to detect LSU-6His in pRST107 and pRST108 transformants and the anti-HA-HRP antibody was used to detect SSU-HA in pRST107 transformants. Seventeen pRST107 transformants were identified that accumulated the 54 kD LSU-6His while 14 pRST107 transformants and 16 pRST108 transformants accumulated the 15.6 kD SSU-HA (FIG. 11). Among pRST107 transformants, 13 plants accumulated both LSU-6His and SSU-HA. In all plants, the detected LSU and SSU had molecular masses matching that expected without the tomato RUBISCO small subunit transit peptide. This indicated that both the LSU-6His and SSU-HA had been translocated into the chloroplast and the transit peptide had been removed from the precursor protein as designed.

Purified C-terminal 6-His tagged GST fusion protein (GST-His) (140 ng) was used as a control when Anti-His (C-term)-HRP antibody was applied, and 14 ng of purified C-terminal HA tagged GST fusion protein (GST-HA) was used as control when Anti-HA-HRP antibody was applied. Concentrations of LSU-6His and SSU-HA were calculated by measuring signal intensities of the control and the sample proteins. In pRST107 transformants, LSU-6His accumulated from 0.04% to 0.1% TSP, while the SSU-HA accumulated to 0.02% -0.3% TSP. In pRST108 transformants SSU-HA accumulated between 0.04% and 0.3% TSP.

### Assembly of RUBISCO Complex

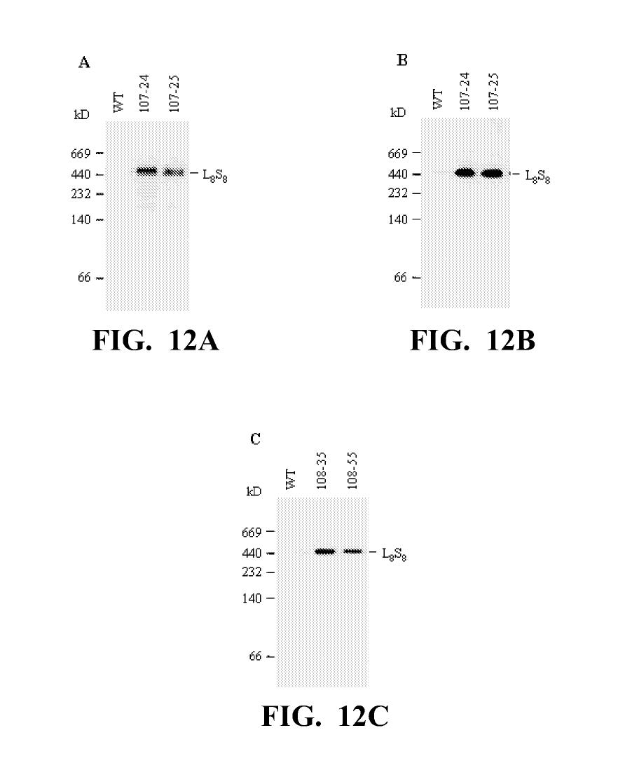

To confirm assembly of A. edulis LSU and SSU in soybean leaves, leaf protein extracts containing 6 μg soluble protein were analyzed by Native-PAGE and Western blot. Anti-His (C-term)-HRP and Anti-HA-HRP antibodies were used to detect LSU-6His in pRST107 and SSU-HA in pRST107 and pRST108 transformants, respectively. The presence of a 550 kD L₈S₈ RUBISCO complex containing LSU-6His and/or the SSU-HA tags in the leaf protein extracts was confirmed (FIG. 12). These results demonstrated that, similar to the case for the tobacco nuclear transformants, the A. edulis SSU had assembled with the soybean endogenous LSU in pRST108 transformants to form a hybrid RUBISCO complex. In pRST107 transformants, soy and A. edulis LSU and SSU may form a mix of hybrid and non-hybrid RUBISCO complexes or they may preferentially assemble only with the subunits from the same species, thus forming a mixture of non-hybrid A. edulis and soy complexes.

### Example 6

### Construction of Amaranthus edulis RUBISCO Transgenes for Chloroplast Expression

In the previous Examples, the recipients of the RUBISCO transgenes were wild type tobacco and soybean which contain a significant amount of endogenous Rubisco. The transgenic plants also accumulated large amounts of endogenous RUBISCO (approximately 40% TSP) and no significant differences in growth rate and photosynthetic activity, between the wild type and the transgenic plants, were observed. In addition, because of the large amount of endogenous Rubisco, it was difficult to measure the activity and the kinetic properties of the foreign RUBISCO in the transgenic plants. To eliminate this problem, the rbcL-KO tobacco plant, which is devoid of any endogenous Rubisco, was used as a recipient for chloroplast transformation.

Since A. edulis rbcL is a chloroplast gene, its sequence did not need to be optimized for chloroplast transformation. The cpAErbcL, encoding the A. edulis LSU with a C-terminal 6-His tag was amplified in a standard PCR reaction using Pfu DNA polymerase and plasmid pRBI104 as the template. The primers were rbc135 (SEQ ID NO:13) and rbc136 (SEQ ID NO:14). The PCR product was cleaned with QIAquick PCR Purification Kit (Qiagen), treated with NcoI, and then purified with QIAquick Gel Extraction Kit (Qiagen) as recommended by the manufacturer. To construct a chimeric chloroplast transgene containing cpAErbcL, pTCP10, containing cpNTrbcL, was digested with EcoRI, treated with Klenow enzyme (Invitrogen) and dNTP supplement, and digested with NcoI, sequentially. Construction of pTCP10, containing cpNTrbcL, is fully described in commonly owned and co-pending U.S. application 61/017422, filed Dec. 28, 2007, incorporated herein by reference.

The treated pTCP10 plasmid was purified using QIAquick Gel Extraction Kit (Qiagen). The PCR-generated cpAErbcL sequence was ligated into the treated pTCP10 plasmid to replace the NTrbcL-6His coding sequence. The resulting plasmid pTCP11 had a chloroplast expression cassette of tobacco psbA promoter with its 5′ UTR (psbA Pro)::cpAErbcL::tobacco rps16 terminator (rps16 Ter). In this chimeric transgene, cpAErbcL is a 1446-nt coding sequence, encoding a 475-aa LSU and a C-terminal 6His-tag (SEQ ID NO:29).

The DNA fragment of psbA Pro::cpAErbcL::rps16 Ter was then isolated from pTCP11 by NotI/SaII digestion and gel purification. It was inserted into a master chloroplast transformation vector pTCP101 (fully described in commonly owned and co-pending U.S. application 61/017422, filed Dec. 28, 2007, incorporated herein by reference) between the NotI and SaII sites in the polylinker region, producing pTCP103 for AErbcL-6His expression in tobacco chloroplasts (FIG. 13).

### Example 7

### Expression of Amaranthus edulis RUBISCO Transgenes in Tobacco Chloroplast Genomes

### Tobacco Chloroplast Transformation

Plasmid pTCP103 was transformed into the tobacco chloroplast genome by standard biolistic bombardment transformation as described below. The rbcL knock out line was grown under sterile conditions using ½ strength Gibco BRL MS medium (Invitrogen) contained in standard Magenta Tissue Culture Boxes (PlantMedia, Bublin, Ohio). Sterile leaves (3-7 cm in length) were excised and one leaf was placed abaxial side up on SAFC modified MS medium (SAFC Biosciences, Lenexa, Kans.) before bombardment. The vitamin composition and concentrations in this medium are as follows (in mg/L): i-inositol, 100; Niacin, 0.5; Pyrixidine HCl, 0.5; and Thiamine, 0.1 on a root induction plate (MS medium with 0.7% agar and 1.0 mL/L 1,000×vitamins). Leaves were bombarded using the Bio-Rad PDS-1000/He Particle Bombardment System with pTCP103 DNA-coated gold particles (average diameter 0.6 em). The tissue was placed about 3 inches from the stopping screen and a burst pressure of 1100 psi was used. For each bombardment, one mg gold particle coated with 0.5 μg DNA was used. After bombardment, the leaf was placed on agarose-solidified and hormone-containing T867 medium (PhytoTechnology, Lenexa, Kans.) with the abaxial side of the leaf in contact with the medium. Two days after incubation on this medium, the leaves were cut into 1-2 cm size squares and placed on fresh T867 medium containing 500 mg/L spectinomycin. The leaf sections were transferred to fresh spectinomycin-containing medium every 10 days. Spectinomycin-resistant calli and shoots were recovered after about 8 weeks on the selection medium. Shoots were generated from the resistant tissue on SAFC modified SM medium containing 500 mg/L spectinomycin. All media were supplied with 8 g/L agar and 30 g/L sucrose. Eleven transformants derived from pTCP103 were maintained on medium with sugar supplement.

### Expression of RUBISCO Transgenes

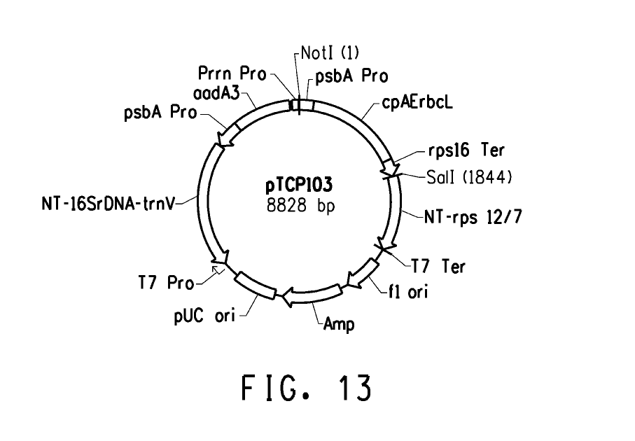

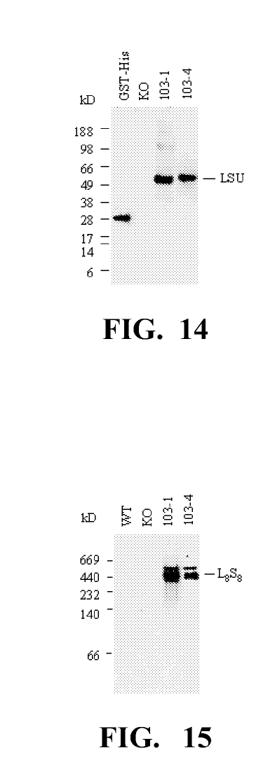

The pTCP103 plant transformed by cpAErbcL was expected to produce a 54 kD A. edulis LSU-6His. To examine the expressions of this gene, TSP extracts were prepared from transformants grown for 2-months on plates, and 5 μg protein samples were analyzed by SDS-PAGE and Western blot. Anti-His (C-term)-HRP was used to detect LSU-6His in pTCP103. Seven of the pTCP103 transformants accumulated the 54 kD LSU-6His (FIG. 14). 140 ng of the purified GST-His was used as control. Concentrations of LSU-6His in the samples were determined as described above. In pTCP103 transformants, average accumulation of the LSU-6His was 1.66% TSP with the highest accumulation of 4% TSP.

### Assembly of RUBISCO Complex

To study assembly of A. edulis LSU in the transplastomic chloroplasts, the leaf protein extracts with transgene expression, containing 5 μg soluble protein, were analyzed by Native-PAGE and Western blot using the antibodies described above. The presence of the 550 kD L₈S₈ RUBISCO complex in leaf protein samples from the pTCP103 transformants was confirmed (FIG. 15). LSU-6His was assembled into the 550 kD L₈S₈ RUBISCO complex. Since in the pTCP103 transformants there is only an A. edulis LSU and no SSU, the presence of the normal-sized complex in these lines indicates that, as in the tobacco and soybean nuclear transformants, the plastome-encoded A. edulis LSU has assembled into a hybrid complex with the tobacco SSU. The Anti-His(C-term)-HRP antibody also recognized an unknown protein or a protein complex somewhat larger than the 550 kD L₈S₈ RUBISCO complex in transformant extracts (FIG. 15).

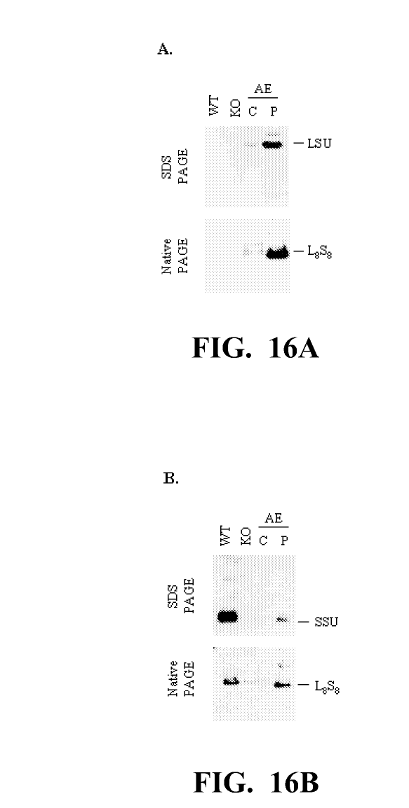

The RUBISCO complex was purified from the pTCP103-1 plant. Leaf tissue (1.0 g) was ground in liquid nitrogen, mixed with 2.5 mL protein extraction buffer (0.1 M NaEPPS pH8.0, 2.5 mM MgCl₂, 0.1 mM EDTA, 10 mM NaHCO₃, 10 mM NaHSO₃, 10 mM 2-mercaptoethanol), and micro-centrifuged twice at 14,000 rpm for 15 min at 4° C. to remove the cell debris. TSP was determined using the Coomassie Plus Protein Assay Reagent (Pierce Colo.). The protein extract was mixed with 0.25 mL Ni-NTA resin (Invitrogen) for 2 h with gentle agitation to bind the 6-His tagged LSU to the resin. The mixture was loaded into a column (0.8 cm in diameter) and allowed to drain. The column was then washed with 8 mL protein extraction buffer. Finally, proteins bound to the column were eluted 4× with 0.4 ml elution buffer (protein extraction buffer with 0.3 M imidazole and 10 mM EDTA). SDS-PAGE analysis of the eluted fractions indicated that both RUBISCO LSU and SSU were bound to the column and released with the elution buffer. The eluted fractions, in addition to Rubisco, contained variable minor amounts of other leaf proteins. Overall, RUBISCO represented about 40% of the total protein in the eluted fraction as estimated subsequently by SDS-PAGE and Coomassie Blue staining. To characterize the proteins purified from the pTCP103-1 transformant, the crude protein extract AE-C (loading fraction, containing 5 μg protein) and purified protein AE-P (eluted fraction, containing approximately 2 μg protein) were analyzed by SDS-PAGE and Native-PAGE Western blot. Wild type tobacco extract containing 2.5 μg protein and rbcL-KO tobacco extract containing 5 μg protein were used as positive and negative controls, respectively. The Western blots were probed by 1,000× diluted Anti-His (C-term)-HRP antibody (Invitrogen) which confirmed that the LSU with a 6-His tag was enriched after purification (FIG. 16A, upper panel). Since LSU-6His protein was located in a 550 kD RUBISCO complex in both crude and purified proteins, the entire complex was enriched along with LSU-6His (FIG. 16A, lower panel). Since neither wild type nor rbcL-KO plants hosted a cpAErbcL transgene, no LSU-6His was detected in the extracts of controls. The Western blots were then probed by 2,000× diluted rabbit antibody produced using spinach rubisco SSU as antigen (Hazelton Biologics, Denver, Pa.) and then by 10,000× diluted Anti-Rabbit IgG-HRP (Jackson ImmunoResearch, West Grove, Pa.) demonstrating that endogenous tobacco RUBISCO SSU had also been enriched during purification of the LSU-6His (FIG. 16B, upper panel). This protein was also present in the purified 550 kD RUBISCO complex (FIG. 16B, lower panel). In the wild type tobacco control, endogenous SSU was also detected. These experiments therefore demonstrated that A. edulis LSU-6His had formed a hybrid L₈S₈ RUBISCO complex in pTCP103 transplastomic tobacco by interacting with the endogenous tobacco RUBISCO SSU.

### Activity of RUBISCO Complex in Transformants

RUBISCO activity in crude extracts of transplastomic plants was determined as described above. Recombinant Rhodospirillum rubrum RUBISCO purified from pRR2119 E. coli strain overexpressing this protein was used as a control. In addition, the crude protein extract and purified RUBISCO of pTCP102 plant 81021 (fully described in commonly owned and co-pending U.S. application U.S. application 61/017422, filed Dec. 28, 2007, incorporated herein by reference) which expressed the tobacco rbcL transgene cpNTrbcL in the rbcL-KO tobacco to a level of 54% total soluble protein and crude protein extracts of the wild-type and rbcL-KO tobacco were also used as positive and negative controls. These analyses (Table 2) confirmed the presence of RUBISCO activity in crude protein extracts of the pTCP103-1 plant. Purification of the RUBISCO complex from these transformants not only enriched the enzyme (FIG. 16) but also concentrated the activity confirming that RUBISCO activity in the transplastomic plants was directly related to expression of A. edulis RUBISCO transgenes. The RUBISCO activity purified from pTCP103-1 transplastomic plant was much lower compared to the pRR2119 control and wild type tobacco Rubisco. This might be due to a natural defect of the hybrid RUBISCO complex. Alternatively, the C-terminal 6His in the transgenic LSU might also contribute to lower activity. However, these activities were also lower than that of pTCP102 plant 81021, which contains the native tobacco LSU with the C-terminal 6His. This implicates the incompatibility between the A. edulis LSU and tobacco SSU subunits as the cause for the reduced activity in the hybrid complex.

TABLE 2
RUBISCO ACTIVITY IN PROTEIN EXTRACTS
Results reported as the average of three measurements

| | RUBISCO Activity | |
|---|---|---|
| Protein Sample | (mU/mg) | Standard Deviation |
| pRR2119 E. coli | | |
| crude extract | 252 | ±16 |
| pTCP103-1 tobacco | | |
| crude extract | 0.15 | ±0.03 |
| purified RUBISCO | 4.6 | ±0.06 |
| pTCP102 (81021) | | |
| tobacco | | |
| crude extract | 43 | ±3.5 |
| purified RUBISCO | 320 | ±33 |
| Wild-type tobacco | | |
| crude extract | 610 | ±50 |
| rbcL-KO tobacco | | |
| crude extract | 0.0 | |

### Example 8

### Construction of Griffithsia monilis RUBISCO Transgenes for Tobacco Nuclear Expression

Plasmid pGm-rbcLS-TVE was used as a template to synthesize a GMNrbcL-6His coding sequence in a standard PCR reaction as described above. The reaction used primer rbc70 (SEQ ID NO:15) and primer rbc71 (SEQ ID NO:16). In this PCR reaction, a 6His-tag coding region was added into the rbcL just before the stop codon and a NotI site was created after the stop codon. At the same time, the first codon (ATG, for Met) was changed to CCA (for Pro) to create an MscI site at the 5′ end. GMNrbcL-6HIS was cloned into pCR-Blunt II-TOPO to produce pTP-GMNrbcL-6HISa. Since GMNrbcL contains additional internal MscI and BamHI sites which would interfere with further cloning procedures, these sites were mutated by using the Quikchange site-direct mutagenesis kit (Stratagene, La Jolla, Calif.), following a protocol provided by manufacture. Initially, the MscI site (TGGCCA) was changed to TGGACA with primer rbc66 (SEQ ID NO:17) and rbc67 (SEQ ID NO:18) to produce pTP-GMNrbcL-MscI. Then, the BamHI (GGATCC) was changed to GGACCC with primer rbc64 (SEQ ID NO:19) and rbc65 (SEQ ID NO:20) producing pTP-GMNrbcLMscI/BamHI. Neither of these changes altered the protein sequences they encoded. After sequencing, it was found that the NotI site after the stop codon was not correct. Thus, it was recreated with rbc70 (SEQ ID NO:15) and rbc80 (SEQ ID NO:21) by PCR, using pTP-GrbcLMscI/BamHI as a template. The PCR product was cloned into pCR-Blunt II-TOPO to produce pTP-GMNrbcL-6HIS. Plasmid pGm-rbcLS-TVE was also used as a template to synthesize a GMNrbcS-HA coding sequence in a standard PCR reaction. The reaction used primer rbc68 (SEQ ID NO:22) and rbc69 (SEQ ID NO:23). In the PCR reaction, an HA-tag and a NotI site were added to GMNrbcS just before and after the stop codon, respectively. The first codon GTG, encoding valine, of GMNrbcS was removed, and an MscI site was added before the second amino acid codon which created a Pro codon CCA. The PCR product was cloned into pCR-Blunt II-TOPO, creating pTP-GMNrbcS-HA. Finally, the GMNrbcL-6HIS and GMNrbcS-HA coding sequences were isolated from pTP-GMNrbcL-6HIS and pTP-GMNrbcS-HA through MscI/NotI digestion, and each was translationally fused to a tomato RUBISCO SSU transit peptide coding sequence in a pBS plasmid, resulting in pBS-nGMNrbcL and pBS-nGMNrbcS, respectively. In pBS-nGMNrbcL, nGMNrbcL (SEQ ID NOs:30 and 31) was a 1,668-bp coding sequence. It encoded a 555-amino acid fusion protein consisting of a tomato transit peptide, a G. monilis RUBISCO LSU, and a C-terminal 6His-tag. In pBS-nGMNrbcS, nGMNrbcS (SEQ ID NOs:32 and 33) was a 627-bp coding sequence. It encoded a 208-amino acid fusion protein consisting of a tomato transit peptide, a G. monilis mature RUBISCO SSU, and a C-terminal HA-tag.

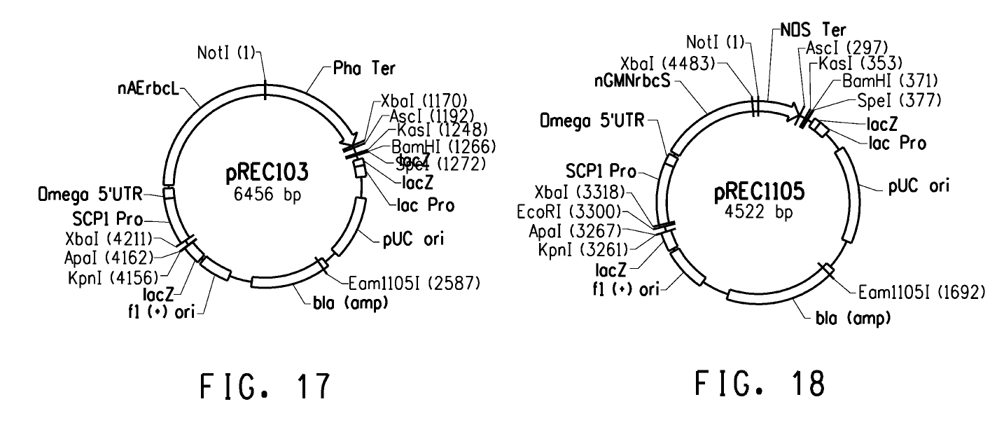

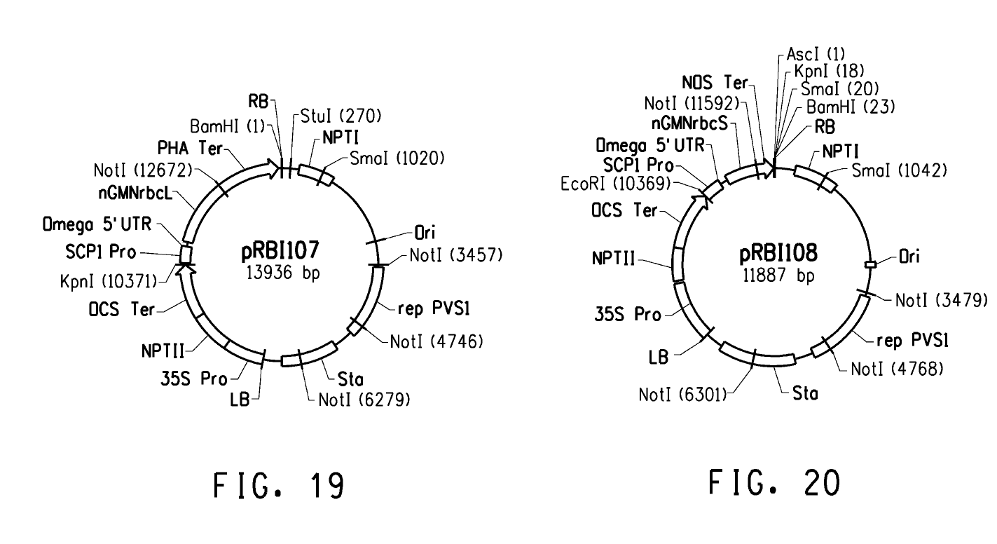

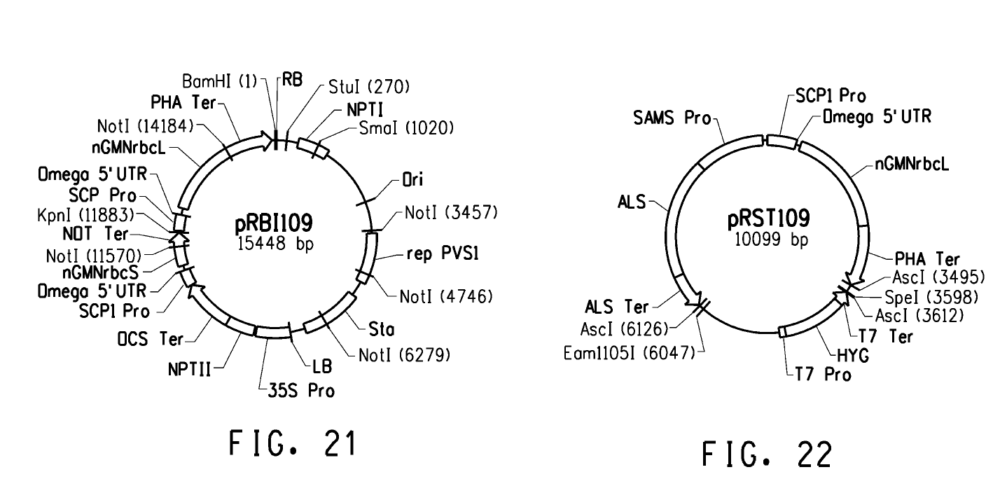

In order to express nGMNrbcL and nGMNrbcS from a plant nucleus, both sequences were isolated from their host pBS plasmids by StuI/NotI digestion and purified using a QIAquick Gel Extraction Kit. Then, the nGMNrbcL was cloned into the pREC10 plasmid between the SmaI and NotI sites. Since pREC10 was a pBS based plasmid containing a synthetic SCP1 promoter with the omega 5′UTR (SCP1 Pro) and a soybean phaseolin terminator (Pha Ter), the cloning generated a vector, pREC103, which harbored the chimeric transgene expression cassette SCP1 Pro::nGMNrbcL::Pha Ter. FIG. 17 shows a map of pREC103. In parallel, nGMNrbcS was cloned into the pREC11 plasmid between the SmaI and NotI sites. The structure of pREC11 was similar to that for pREC10, except the Agrobacterium T-DNA NOS terminator (NOS Ter) was substituted for Pha Ter. The resultant plasmid, termed pREC1105 (FIG. 18), contained the chimeric transgene expression cassette SCP1 Pro::nGMNrbcS::NOS Ter. These two chimeric transgenes were then inserted into a pZBL1M1-based expression plasmid for tobacco nuclear genome transformation. pZBL1M1 is a master tobacco binary vector containing the marker gene CaMV 35S promoter (35S Pro)::NPTII::Agrobacterium T-DNA OCS terminator (OCS Ter). When making the constructs, a standard digestion, gel-purification, and sub-cloning procedure was followed. SCP1 Pro::nGMNrbcL::Pha Ter was isolated from pREC103 by KpnI/BamHI digestion and inserted into pZBLM1 to form pRBI107 (FIG. 19). SCP1 Pro::nGMNrbcS::NOS Ter was isolated from pREC1105 by EcoRI/AscI digestion and inserted into pZBLM1 to form pRBI108 (FIG. 20). SCP1 Pro::nGMNrbcL::Pha Ter isolated earlier from pREC103 by KpnI/BamHI digestion was inserted into pRBI108 to form pRBI109 (FIG. 21).

### Example 9

### Expression of Griffithsia monilis RUBISCO Transgenes in Tobacco Nuclear Genomes

### Tobacco Nuclear Transformation

Plasmids pRBI107, pRBI108, and pRBI109 were introduced into wild type Nicotiana tabacum nuclear genomes by a standard Agrobacterium-mediated transformation approach. In the first step of the procedure, 1.0 μg plasmid DNA was electroporated into competent cells of Agrobacterium strain LBA4404 (Invitrogen) in a 2 mm cuvette at 2.5 kV using a TransPorator Plus device (BTX, San Diego, Calif.). Agrobacterium cells were grown overnight in MinA medium (1% Bacto tryptone, 1% yeast extract, 0.5% NaCl) containing 50 mg/L kanamycin), washed with MS medium (Gibco BRL MS salts with 3% sucrose), and resuspended in a double volume of MS. In the second step, sterile wild type tobacco leaf discs (one cm in diameter) were infected by incubating in the Agrobacterium suspension for 30 min, transferred onto a shoot induction plate (MS medium with 0.7% agar, 0.1 mg/L NAA, 1 mg/L BAP, and 1 mL/L 1,000× vitamins) for 3 days, washed in 30 mL MS medium containing 500 mg/L cefotaxime (Calbiochem, San Diego, Calif.) for 20 min, and then placed on a shoot induction plate containing 300 mg/L kanamycin and 500 mg/L cefotaxime for 3 weeks. Leaf discs were transferred to a new shoot induction plate to allow callus growth and shoot regeneration. Finally, roots were regenerated as described above. A total of 13 independent transformants for PRBI107, 23 for PRBI108, and 21 for PRBI109 were obtained. These were transplanted into Metro Mix soil and grown in plant growth chambers under a 16/8 h photoperiod at 26/24° C. day/night temperatures.

### Expression of RUBISCO Transgenes

Based on its transgene structure, the pRBI107 transformants should produce a 61.3 kD precursor that should be imported into chloroplasts and processed into a 55.6 kD 6-His tagged G. monilis rubisco LSU (i.e. LSU-6His), a chloroplast-accumulated product of nGMNrbcL. Similarly, the pRBI108 transformants should produce a 23.5 kD precursor that should be imported into chloroplasts and processed into 17.7 kD HA tagged G. monilis RUBISCO SSU (i.e. SSU-HA), a chloroplast-accumulated product of nGMNrbcS. The pRBI109 plant should produce both the 6-His tagged G. monilis RUBISCO LSU and HA tagged G. monilis RUBISCO SSU. To examine expression of these proteins, total soluble protein extract was prepared by grinding 150 mg leaves of transformants grown for one month in soil in 200 uL ice-cold leaf extraction buffer. The buffer contained 50 mM Tris-HCl at pH 8.0, 50 mM NaCl, 0.1 mM EDTA, 2 mM DTT, 5 mM MgCl₂, 5% glycerol, and 1% protease inhibitor cocktail for plant (Sigma, St. Louis, Mo.). Cell debris was removed by centrifugation at 10,000×g at 4° C. for 15 min. Protein concentration in the supernatant was determined using the Coomassie Plus Protein Assay Reagent (Pierce, Rockford, Ill.). Protein extracts containing 6 ug total soluble protein were subjected to SDS-PAGE on a 4%-12% NuPAGE Novex Bis-Tris Gel (Invitrogen, Carlsbad, Calif.). Sample pre-treatment and electrophoresis were conducted using NuPAGE reagents and following the NuPAGE Technical Guide (Invitrogen). The separated proteins were transferred from the NuPAGE SDS gel to a nitrocellulose membrane (Invitrogen) using a Pharmacia-LKB 2117 multiphor 11 (Pharmacia Biotech, Piscataway, N.J.), sandwiched by 2 layers of Whatman 1 filter paper on the both sides. The gel and filter were moistened with semi-dry western transfer buffer (40 mM glycine, 50 mM Tris, 1 mM SDS, and 20% methanol). The transfer was carried out at 0.8 mA/cm² for 1.5 h. Protein blots were probed with 1,000× diluted Anti-His (C-term)-HRP Antibody (Invitrogen) for LSU-6His (in pRBI107 and pRBI109 transformants) and with 5,000× diluted Anti-HA-HRP (Sigma) for SSU-HA (in pRBI108 and pRBI109 transformants). Signals were detected with SuperSignal West Pico Chemiluminescent Substrate Solution (Pierce) in a standard western blot assay. The results, recorded using a Lumi-Imager (Roche Diagnostics, Indianapolis, Ind.), indicated that none of these transformants accumulated the transgene products in the soluble protein fraction (data not shown). Therefore, G. monilis rbcL and rbcS genes could not be produced in tobacco through a nuclear transformation approach, although their codon usage was similar to higher plant nuclear genes.

### Example 10

### Construction of Griffithsia monilis RUBISCO Transgenes for Soybean Nuclear Expression

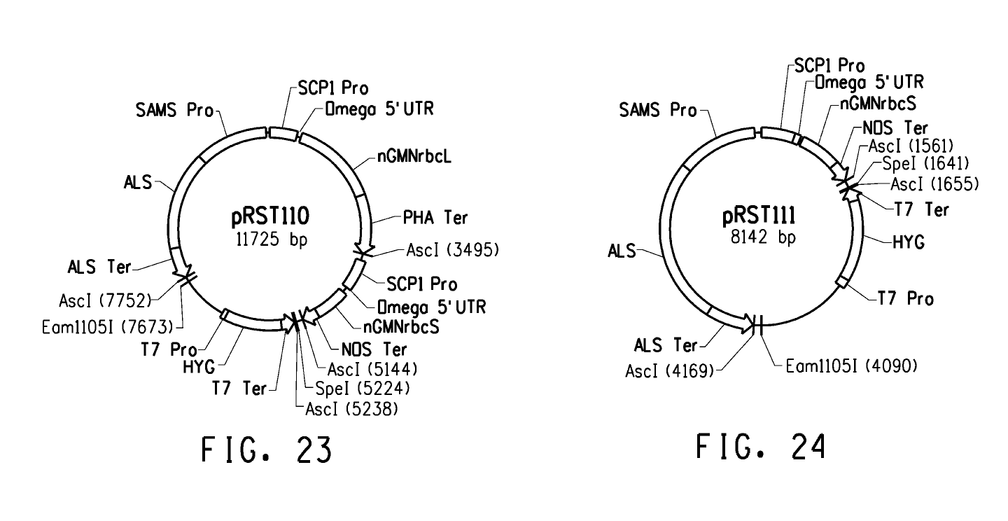

To achieve soybean nuclear expression of G. monilis RUBISCO transgenes, a chimeric transgene expression cassette containing SCP1 Pro::nGMNrbcL::Pha Ter in pREC103 and a chimeric transgene expression cassette containing SCP1 Pro::nGMNrbcS::NOS Ter in pREC1105 were inserted into the pZSL222-based expression plasmid. The pZSL222 vector is a master soybean expression vector containing a marker gene, soybean SAMS promoter (SAMS Pro)::ALS::soybean ALS terminator (ALS Ter). When making the constructs, a standard digestion, gel-purification, and sub-cloning procedure was followed. First, SCP1 Pro::nGMNrbcL::Pha Ter was isolated from pREC103 by ApaI/NotI digestion and inserted into pZSL222 to form pRST109 (FIG. 22). Then, SCP1 Pro::nGMNrbcS::NOS Ter was isolated from pREC1105 by KpnI/SpeI digestion and inserted into pRST109 to form pRST110 (FIG. 23), which contained both nGMNrbcL and nGMNrbcS transgenes. Finally, SCP1 Pro::nGMNrbcS::NOS Ter isolated from pREC1105 by ApaI/SpeI digestion was inserted into pZST222 to form pRST111 (FIG. 24).

### Example 11

### Expression of Griffithsia Monilis RUBISCO Transgenes in Soybean Nuclear Genomes

### Soybean Nuclear Transformation

Plasmids pRST110 and pRST111 were introduced into soybean nuclear genomes by a standard biolistic bombardment transformation approach (Finer and McMullen, 1991; Stewart et al., 1996). Briefly, embryogenic suspension cultures of Glycine max Merrill (cultivar “Jack”) were maintained in 250 ml flasks containing 50 ml of liquid MS medium on rotary shakers at 26° C. under cool white fluorescent lights with a 16/8 hour day/night photoperiod. Freshly subcultured cultures were bombarded using the Bio-Rad PDS-1000/He Particle Bombardment System (Bio-Rad, Hercules, Calif.) with plasmid DNA-coated gold particles (average diameter 1.0 em). A burst pressure of 1100 psi was used. The tissue was placed about 3.5 inches from the stopping screen. For each bombardment, 0.3 mg gold particle coated with 0.03 μg DNA was used. After bombardment, the tissue was put back into the liquid MS medium and cultured for 7 days. Then, the liquid medium was replaced by a liquid MS medium containing 100 ng/mL chlorsulfuron as selection agent. This selective medium was refreshed weekly, until green transformed tissue was observed. The isolated transformed tissue was cultured in a liquid MS medium containing 100 ng/mL chlorsulfuron and 10 mg/mL 2,4-dichlorophenoxyacetic acid (2,4-D) and further on a solid MS medium containing 2,4-D to allow embryos to develop. It was placed on a solid germination medium to regenerate plantlets of transgenic soybean. Finally, a total of 9 independent transformants for pRST110 and 13 for pRST111 were obtained. These were transplanted into Metro Mix soil and grown in a growth chamber under 16/8 h photoperiod at 26/24° C. day/night temperatures.

### Expression of RUBISCO Transgenes

Transgene insertion in all soybean pRST110 and pRST111 transformants were confirmed by PCR analysis. Since pRST110 plants were transformed by both nGMNrbcS and nGMNrbcL, they should produce both G. monilis RUBISCO LSU-6His and SSU-HA., whereas the pRST111 transformants should produce only the SSU-HA because they possessed only the nGMNrbcS transgene. To examine transgene expression, soluble protein extracts were prepared from transformants grown for one month in soil, and 6 μg protein was subjected to SDS-PAGE and Western blot analyses, as described above. Anti-His (C-term)-HRP Antibody was used to detect LSU-6His in pRST110 transformants. Anti-HA-HRP Antibody was used to detect SSU-HA in pRST110 and pRST111 transformants. The results, recorded by Lumi-Imager, indicated that none of these transformants accumulated the RUBISCO transgene products (data not shown). This further confirmed that G. monilis RUBISCO could not be produced in higher plants through a nuclear transformation approach.

### Example 12

### Construction of Griffithsia monilis RUBISCO Transgenes for Chloroplast Expression

In previous Examples, transformation of G. monilis RUBISCO genes into tobacco and soybean nuclear genomes did not result in accumulation of transgene products in these transformants. Chloroplast transformation of rbcL-KO tobacco was therefore performed as an alternative approach to achieve accumulation of the RUBISCO enzyme.

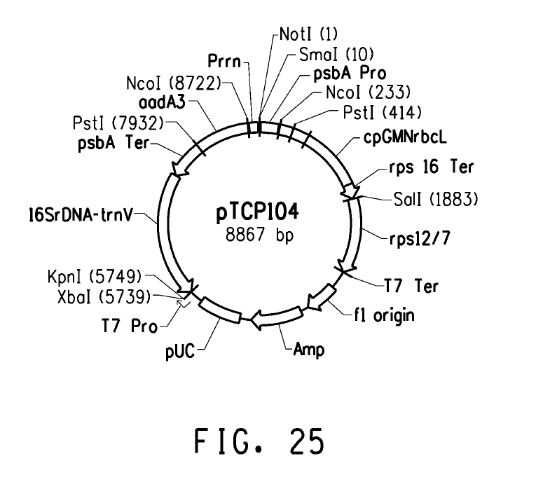

Since G. monilis rbcL is a chloroplast gene its sequence did not need to be optimized for chloroplast transformation. The cpGMNrbcL, encoding the G. monilis LSU with a C-terminal 6-His tag was synthesized in a standard PCR reaction using Pfu DNA polymerase with plasmid pRBI107 as the template. The PCR primers were rbc134 (SEQ ID NO:24) and rbc136 (SEQ ID NO:14). The PCR product was cleaned with the QIAquick PCR Purification Kit (Qiagen), treated with NcoI, and then purified with QIAquick Gel Extraction Kit (Qiagen). To construct a chimeric chloroplast transgene of cpGMNrbcL, pTCP10 (as described in commonly owned and copending application U.S. application 61/017422, filed Dec. 28, 2007, incorporated herein by reference) containing cpNTrbcL, was digested with EcoRI, treated by Klenow enzyme (Invitrogen) with dNTP supplement, and digested with NcoI, sequentially. The treated pTCP10 plasmid was purified using a QIAquick Gel Extraction Kit (Qiagen). The PCR-generated cpGMNrbcL sequence was integrated into the treated pTCP10 plasmid to replace the NTrbcL-6His coding sequence by ligation. The resultant plasmid pTCP12A had a chloroplast expression cassette containing tobacco psbA promoter with its 5′ UTR (psbA Pro)::cpGMNrbcL::tobacco rps16 terminator (rps16 Ter). In this chimeric transgene, cpGMNrbcL is a 1485-nt coding sequence, encoding a 488 residue LSU and a C-terminal 6His-tag (SEQ ID NOs: 34 and 35). Finally, a DNA fragment of psbA Pro::cpGMNrbcL::rps16 Ter was isolated from pTCP12A by NotI/SaII digestion and gel purification. It was inserted into a master chloroplast transformation vector pTCP101 (as described in commonly owned and copending application U.S. application 61/017422, filed Dec. 28, 2007, incorporated herein by reference) between NotI and SaII in the polylinker region, resulting in pTCP104 for GMNrbcL-6His expression in tobacco chloroplasts. A map of pTCP104 is shown in FIG. 25.

### Example 13

### Expression of Griffithsia monilis RUBISCO Transgenes in Tobacco Chloroplast Genomes

The rbcL-KO tobacco plant was chosen as a recipient of the cpGMNrbcL transgene. This plant was developed by Icon Genetics (Halle, Germany) through a research contract with Verdia/Pioneer (as described in commonly owned and copending application U.S. application 61/017422, filed Dec. 28, 2007, incorporated herein by reference). In the chloroplast genome of this plant, the majority of the rbcL coding sequence was replaced with a GFP gene. An rbcL fragment that encoded the N-terminal 59 amino acids was translationally fused with the GFP. Thus, there was no functional rbcL gene in the rbcL-KO genome. The plant accumulated neither LSU nor SSU protein, had no RUBISCO activity, and no photosynthesis activity. The homoplastomic rbcL-KO plant appeared pale and only survived when sugar was provided. Chimeric WT/rbcL-KO plants were able to grow without sugar supplement since the WT leaf sectors could photosynthesize and cross-feed the mutant sectors.

### Tobacco Chloroplast Transformation

Plasmid pTCP104 was transformed into the tobacco chloroplast genome by a standard biolistic bombardment transformation approach. For this purpose, the rbcL-KO line was grown under sterile conditions using ½ strength MS medium (Gibco BRL) in standard Magenta tissue culture boxes. Sterile leaves (3-7 cm in length) were excised and one leaf was placed abaxial side up on SAFC modified MS medium (SAFC Biosciences, Lenexa, Kans.) before bombardment. Leaves were bombarded using the Bio-Rad PDS-1000/He Particle Bombardment System with pTCP104 DNA-coated gold particles (average diameter 0.6 μm). A burst pressure of 1100 psi was used and the tissue was placed about 3 inches from the stopping screen. For each bombardment, 1 mg gold particle coated with 0.5 μg DNA was used. After bombardment, the leaf was placed on bactoagar-solidified and hormone-containing T867 medium (PhytoTechnology, Lenexa, Kans.) with the bottom surface of the leaf in contact with the medium. Two days after incubation on this medium, the leaves were cut into 1-2 cm squares and placed onto fresh T867 media containing 500 mg/L spectinomycin. The leaf sections were transferred to fresh spectinomycin-containing medium every 10 days. Spectinomycin-resistant calli and shoots were recovered after about 8 weeks on the selection medium. Shoots were generated from the resistant tissue on SAFC modified SM medium containing 500 mg/L spectinomycin. All media were supplied with 8 g/L bactoagar and 30 g/L sucrose. A total of 11 independent transformants were obtained. These plants were maintained on the SAFC modified SM medium with sugar supplement.

### Expression of RUBISCO Transgenes

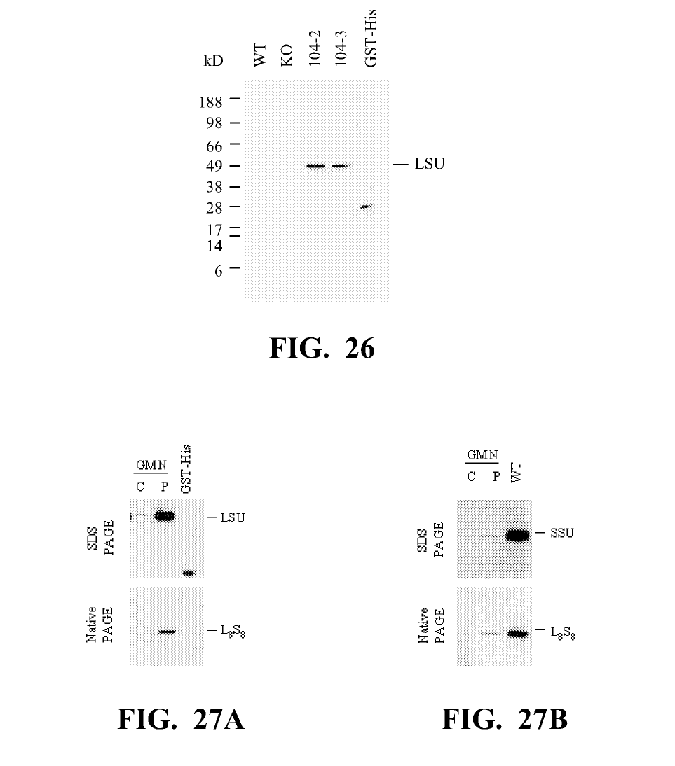

Transgene insertions in all transformants of pTCP104 were confirmed by PCR analysis. The pTCP104 plant containing the cpGMNrbcL transgene should produce a 55 kD G. monilis LSU-6His. To examine the transgene expression, total soluble protein extract was prepared from transformants grown for two months on plates, and samples containing 5 ug protein were subjected to SDS-PAGE and Western blot assay, as described above. Anti-His (C-term)-HRP Antibody was used to detect LSU-6His. The results, recorded by Lumi-Imager, showed that 9 out of 11 pTCP104 transformants accumulated the 55 kD LSU-6His. The results for two transformants (pTCP104-2 and pTCP104-3) are presented in FIG. 26. In the assay, 140 ng of purified GST-His was used as a quantification control protein. By measuring signal intensities of control protein and sample proteins, accumulation levels of LSU-6His were calculated. The average accumulation of the LSU-6His was 0.04% TSP with a highest accumulation of 0.19% TSP. Therefore, through a chloroplast transformation approach, soluble G. monilis RUBISCO LSU subunit was expressed and accumulated in tobacco chloroplasts at a low but detectable level.

### Assembly of RUBISCO Complex

RUBISCO complex assembly in the transformants was studied by purifying the complex from the pTCP104-1 transformant (originally named the 81221 transformant). For this purpose, 1 g leaf tissue was ground in liquid nitrogen, mixed with 2.5 mL protein extraction buffer (0.1 M NaEPPS pH8.0, 2.5 mM MgCl₂, 0.1 mM EDTA, 10 mM NaHCO₃, 10 mM NaHSO₃, 10 mM 2-mercaptoethanol, then micro-centrifuged twice at 14,000 rpm for 15 min at 4° C. to remove cell debris. The concentration of total soluble protein was determined using the Coomassie Plus Protein Assay Reagent (Pierce). The protein extract was mixed with 0.25 mL Ni-NTA resin (Invitrogen) for 2 h with gentle agitation to bind 6-His tagged LSU to the resin. The mixture was loaded on a column to collect flow through fractions and then washed with 8 mL protein extraction buffer. Finally, proteins on the column were eluted 4 times with 0.4 ml elution buffer (protein extraction buffer with 0.3 M imidazole and 10 mM EDTA), with each elution fraction collected separately. SDS-PAGE analysis indicated that the purification was not efficient due to the low level of expression and non-RUBISCO leaf proteins were present in the eluted fractions.

To characterize the proteins purified from the pTCP104-1 plant, crude soluble protein extract GMN-C (loading fraction, containing 5 μg protein) and purified protein GMN-P (elution fraction, containing approximately 2 μg protein) were subjected to SDS-PAGE and Native-PAGE Western blot assays, as described earlier. Initially, the Western blots, were probed by 1,000× diluted Anti-His (C-term)-HRP Antibody (Invitrogen). Results of the SDS-PAGE Western blotting (FIG. 27A, upper panel) showed that the 55 kD LSU-6His was barely detectable in the crude extract GMN-P and but was substantially enriched after purification. Native-PAGE Western blotting (FIG. 27A, lower panel) demonstrated that the LSU-6His protein co-migrated with the authentic 550 kD L₈S₈ RUBISCO complex. When the LSU-His was purified, the complex was enriched along with it and became detectable. In this Native-PAGE, the positive control protein GST-His migrated near the bottom of gel and thus is not shown in FIG. 27.

A second set of Western blots, which used wild type tobacco extract containing 2.5 μg protein as a positive control, were probed by 2,000× diluted Anti-rbcS Antibody, produced using spinach RUBISCO SSU as antigen (Hazelton Biologics, Denver, Pa.) and then by 10,000× diluted Anti-Rabbit IgG-HRP (Jackson ImmunoResearch, West Grove, Pa.). Results (FIG. 27B) indicated that endogenous tobacco RUBISCO SSU was enriched during the purification of the LSU-6His (upper panel) and became detectable after the purification process. This protein was also located in the purified 550 kD RUBISCO complex (lower panel), as in the wild type tobacco control. Therefore, these experiments demonstrated that, in pTCP104 transplastomic tobacco, G. monilis LSU-6His formed a hybrid L₈S₈ RUBISCO complex by interacting with the endogenous tobacco RUBISCO SSU.

### Activity of RUBISCO Complex in Transformants

To demonstrate RUBISCO activity in the transplastomic plants, the crude protein extracts and the purified RUBISCO complexes obtained from pTCP104-1 plants were dialyzed at 4° C. overnight against a solution consisting of 0.1 M NaEPPS pH 8.0, 2.5 mM MgCl₂, 0.1 mM EDTA, 10 mM NaHCO₃, 10 mM NaHSO₃, and 10 mM 2-mercaptoethanol. They were then assayed for RUBISCO activity by measuring ribulose-1,5-bisphosphate (RuBP) dependent ¹⁴CO₂ fixation. The RUBISCO was first activated by addition of MgCl₂ and NaHCO₃ to 20 mM each, and incubated at room temperature for one h. Reactions were run in 30 μl total volume in 1.5 ml polypropylene tubes. The mixture consisted of 15 μl extract (diluted as needed with 0.1 M NaEPPS, pH 8, containing 20 mM MgCl₂, 20 mM NaHCO₃, 1.0 mM EDTA, 50 μg/ml bovine serum albumin, and 2 mM dithiothreitol). Ten microliters of a solution of [¹⁴C]—NaHCO₃ (ca. 0.3 mM) was added. Reaction at 25° C. was started by addition of 5 μL 6 mM RuBP. Three assays containing different levels of each highly active extract were performed for 10 min, after which 25 μL of the reaction was transferred to a 7 mL glass vial containing 0.4 ml 10% v/v acetic acid. Two pairs of reactions were run for less active samples. Each reaction containing RuBP was paired with another lacking RuBP, and reactions were terminated at 10 min and 60 min. Three controls with excess enzyme for determination of the specific radioactivity of ¹⁴C in the assay and three controls with no enzyme were performed with each set of assays. The vials containing quenched reactions were taken to dryness on a hotplate, and taken up in 0.2 mL water. Ecolume scintillation fluid (5 mL, MP Biologicals, Solon, Ohio) was added, and the samples were capped and counted in a Beckman LS6000TA liquid scintillation counter. The specific activity of the ¹⁴C in the assay was calculated by subtracting the mean of the no-enzyme controls from the excess enzyme controls, averaging the result, and dividing by 25 nmol RuBP originally present in the volume of reaction transferred into the acetic acid quench. For samples with high activity, the no-enzyme value was subtracted from the observed counts, and the corrected value converted to nmol ¹⁴C fixed. For less-active samples, the counts in the −RuBP reaction of each pair was subtracted from the corresponding +RuBP reaction. If the difference was considered meaningful (at least 50% higher +RuBP, in both samples), nmol ¹⁴C fixed were calculated as above. The results (Table 3) were then converted to nmoles/min/mg of protein (mU/mg) taking into account the volume of extract in each assay. A crude extract of E. coli containing pRR2119, which expresses recombinant Rhodospirillum rubrum RUBISCO (Somerville and Somerville, Mol Gen Genet 193, 214-219, 1984), was used as a control, representing standard RUBISCO activity in the assay. The assay data confirmed that crude protein extracts of pTCP104-1 had no detectable RUBISCO activity due to low expression level. However, the purification not only enriched the holoenzyme but also concentrated the activity, demonstrating that RUBISCO activity in the transplastomic plants was directly related to expression of the G. monilis RUBISCO transgene. RUBISCO activity in the purified elution fraction of the pTCP104-1 transplastomic plant was quite low compared to the pRR2119 control here or other transplastomic plants described earlier (Table 2). This likely was due to the low expression level of G. monilis RUBISCO LSU and the poor compatibility between G. monilis RUBISCO LSU and endogenous tobacco RUBISCO SSU in forming a hybrid complex.

TABLE 3
RUBISCO ACTIVITY IN PROTEIN EXTRACTS

| | RUBISCO Activity |
|---|---|
| Protein Sample | (mU/mg) |
| pRR2119 E. coli | |
| crude extract | 510 |
| pTCP104-1 tobacco | |
| crude extract | 0.0 |
| purified rubisco | 2.1 |
| rbcL-KO tobacco | |
| crude extract | 0.0 |

What is claimed is:

1. A method for the recombinant expression of an L8S8 RUBISCO enzyme in a plant cell comprising:
a) providing a C3 plant cell comprising a transformation vector wherein the vector comprises a heterologous genetic construct encoding a plant protein selected from the group consisting of: the small subunit of a L8S8 RUBISCO enzyme and the large subunit of an L8S8 RUBISCO enzyme, wherein the large and small subunits of the L8S8 RUBISCO enzyme are derived from a C4 plant or a rhodophyte; and
b) growing the plant cell under conditions whereby the protein is expressed in soluble form.

2. The method of claim 1 wherein the vector is a nuclear transformation vector and where the genetic construct encoding the plant protein is located in the plant cell nucleus.

3. The method of claim 1 wherein the vector is a chloroplast transformation vector and where the genetic construct encoding the plant protein is expressed in the plant cell chloroplast.

4. The method of claim 1 wherein the genetic constructs encoding the L8S8 RUBISCO large subunits are isolated from a genus selected from the group consisting of Amaranthus, Zea, Saccharum and Griffithsia.

5. The method of claim 1 wherein the genetic constructs encoding the L8S8 RUBISCO small subunits are isolated from a genus selected from the group consisting of Amaranthus, Zea, Saccharum and Griffithsia.

6. The method of claim 4 wherein the genetic construct encodes an L8S8 RUBISCO large subunit having at least 90% identity to the amino acid sequence selected from the group consisting of SEQ ID NO: 31, 35, 36, 38, and 40 using the Clustal W method of alignment.

7. The method of claim 5 wherein the genetic construct encodes an L8S8 RUBISCO small subunit having at least 90% identity to the amino acid sequence selected from the group consisting of SEQ ID NO: 33, 37 and 39 using the Clustal W method of alignment.

8. The method of claim 4 wherein the genetic construct comprises a nucleic acid sequence encoding a RUBISCO large subunit which hybridizes under the stringent conditions of 0.1×SSC, 0.1% SDS, 65° C. and washed with 2×SSC, 0.1% SDS followed by 0.1×SSC, 0.1% SDS to a nucleic acid sequence selected from the group consisting of SEQ ID NO:'s 25, 27, 29, 30 and 34.

9. The method of claim 5 wherein the genetic construct comprises a nucleic acid sequence encoding a RUBISCO small subunit which hybridizes under the stringent conditions of 0.1×SSC, 0.1% SDS, 65° C. and washed with 2×SSC, 0.1% SDS followed by 0.1×SSC, 0.1% SDS to a nucleic acid sequence selected from the group consisting of SEQ ID NO;'s 26, 28 and 32.

10. The method of claim 1 wherein the C3 plant is selected from the group consisting of tobacco, soybean, rice, canola, cotton and wheat.

11. The method of claim 3 wherein the vector consists essentially of the general structure:
hetero Pro1::M::Tern hetero Pro2::RBC::Ter2
Wherein:
a) hetero Pro1 is a promoter derived from a non-RUBISCO plant gene;
b) M genetic construct encoding a selectable marker;
c) Tern is a terminator;
d) hetero Pro2 is a promoter derived from a non-RUBISCO plant gene;
e) RBC is a genetic construct encoding a plant protein selected from the group consisting of: the small subunit of a L8S8 RUBISCO enzyme and the large subunit of an L8S8 RUBISCO enzyme, wherein the large and small subunits of the L8S8 RUBISCO enzyme are derived from a C4 plant or a rhodophyte; and
f) Ter2 is a terminator.

12. The method of claim 11 wherein hetero Pro1 is a promoter derived from a plastid rRNA operon.

13. The method of claim 11 wherein Ter1 is a terminator derived from a plastid psbA gene.

14. The method of claim 11 wherein Ter2 is a terminator derived from the plastid rps16 gene.

15. The method of claim 11 wherein the vector optionally comprises a first flanking homology arm and a second flanking homology arm for integration of the vector into the chloroplast genome.

16. A C3 plant comprising a soluble plant protein selected from the group consisting of: the small subunit of a L8S8 RUBISCO enzyme and the large subunit of an L8S8 RUBISCO enzyme, wherein the large and small subunits of the L8S8 RUBISCO enzyme are derived from Amaranthus or Griffithsia.

17. A polypeptide encoding a large subunit of an L8S8 RUBISCO enzyme selected from the group consisting of SEQ ID NO: 36, 38 and 40.

18. An isolated nucleic acid sequence encoding the polypeptide of claim 17.

19. An isolated nucleic acid sequence encoding a large subunit of an L8S8 RUBISCO enzyme having a nucleic sequence selected from the group consisting of SEQ ID NO: 25, 27 and 29.

20. A polypeptide encoding a small subunit of an L8S8 RUBISCO enzyme selected from the group consisting of SEQ ID NO: 37 and 39.

21. An isolated nucleic acid sequence encoding the polypeptide of claim 20.

22. An isolated nucleic acid sequence encoding a small subunit of an L8S8 RUBISCO enzyme having a nucleic sequence selected from the group consisting of SEQ ID NO: 26 and 28.
